SEQUENCE LISTING (92 sequences)

SEQ ID NO: 1 - DNA, 1146 bp, Lysinibacillus sphaericus
  CDS (1)..(1146): phenylalanine dehydrogenase
atggcaaagcagcttgagaagtcctcgaagatcggcaatgaagatgtgttccagaagattgcgaaccatgaacagatcgtattctgtaatgaccctgtgtctggtctgcaggcgattatcgcaatccacgataccacgttgggccctgctctgggcggaactcgcatgtacccgtacaagaacgtggatgaagcgttggaggatgtcctgcgcctttctgagggaatgacctacaagtgtgccgctgcagacatcgattttggcggtggtaaagccgttatcatcggcgacccagagaaggataaatccccagccttgtttcgcgcattcggtcagtttgtcgaatccctgaatgggcgcttctataccggcactgacatgggaactaccatggacgactttgtgcacgcacagaaggaaaccaacttcatcaacggtatcccagagcaatacggaggtagcggagatagcagcattcctacagcgcagggtgtcatttatgctctgaaggccaccaatcagtacctgttcggctctgactccttgtccggtaagacgtacgcaattcaggggctcggtaaagtcggctacaaggtggctgagcaactcctcaaagcaggcgctgatctctttgtcaccgacattcacgaaaacgtcctgaattcgatcaagcagaagtccgaggaacttggtggctccgttactatcgtaaagtcagacgatatttactcggtacaagccgacatcttcgttccatgcgctatgggaggcatcatcaacgacaagaccattcccaaactgaaagtgaaagcggttgttggctcagcgaacaaccagctgaaggatctgcgtcatgccaacgttctgaacgaaaagggcatcctgtatgctccggattacatcgtcaacgctggagggctcattcaggtggcagacgagctttacggtccgaacaaagagcgggtgctccttaagacgaaggagatttatcgctccctcttggaaatcttcaaccaagcagctcttgattgcatcacaaccgttgaagcagccaaccgaaaatgccagaaaaccatcgaaggccaacaaacccggaactctttcttctcccgtgggcgtcgccccaagtggaatatcaaagagtaa

SEQ ID NO: 2 - protein, 381 aa, Lysinibacillus sphaericus
MAKQLEKSSKIGNEDVFQKIANHEQIVFCNDPVSGLQAIIAIHDTTLGPALGGTRMYPYKNVDEALEDVLRLSEGMTYKCAAADIDFGGGKAVIIGDPEKDKSPALFRAFGQFVESLNGRFYTGTDMGTTMDDFVHAQKETNFINGIPEQYGGSGDSSIPTAQGVIYALKATNQYLFGSDSLSGKTYAIQGLGKVGYKVAEQLLKAGADLFVTDIHENVLNSIKQKSEELGGSVTIVKSDDIYSVQADIFVPCAMGGIINDKTIPKLKVKAVVGSANNQLKDLRHANVLNEKGILYAPDYIVNAGGLIQVADELYGPNKERVLLKTKEIYRSLLEIFNQAALDCITTVEAANRKCQKTIEGQQTRNSFFSRGRRPKWNIKE

SEQ ID NO: 3 - protein, 380 aa, Bacillus
MAKQLEKSSIGNEDIFQKIANHEQVVFCNDPASGLQAIIAIHDTTLGPALGGTRMYPYKSVDEALEDVLRLSEGMTYKCAAADIDFGGGKAVIIGDPEKDKSPALFRAFGQFVESLNGRFYTGTDMGTTMDDFVHAQKETNFINGIPEQYGGSGDSSIPTSKGVVYALKATNQYLFGNDSLSGKTYAIQGLGKVGYKVAEQLLEAGAKIYVTDIHESVLHSIKEKSKELGGSVIIVKSDEIYGVDADVFVPCAMGGIINDDTIPKLKVKAVVGSANNQLKNLRHANVLNEKGILYAPDYIVNAGGLIQVADELYGTNKERVLLKTKEIYRSLLEIFKQADLDCITTVEAANRKCQKTIEDQRNRNSFFSRGRRPKWNIKE

SEQ ID NO: 4 - protein, 377 aa, Virgibacillus alimentarius
MKQLAKKNEQDIFQKIAGHEQVVFCNDPITGLQAIIAIHDTTLGPGLGGTRMYPYENVDDALEDALRLSEGMTYKCAAADIDFGGAKAVIIGDPDKDKTPAMFRKFAQYVDSLNGRFYTGTDMGTTMDDFVQALKETNFINGIPEAYGGSGDSSIPTSQGVVYAIQATNKHVFGDDSLGGKTYAIQGLGKVGYKVAEHLLQAGADLFVTDIHEHVLESIQDKAKELGGSVTVVGSDEIYSVDADVFVPCAMGAVLNDETIPQLTVKAVVGSANNQLKAKTHAQVLADKGILYAPDYIVNAGGLIQVADELYEPNKERVLLKTKAIYDSLLEIYEQAKLADITTEEAANRKCQQTIDEQRNRNNFFARDRRPKWNIRG

SEQ ID NO: 5 - protein, 380 aa, Oceanobacillus limi
MNQQLMKKTQVEPDMFQKIAGHEQVVFCNDPASGLQAIIAIHNTTLGPALGGTRMYPYASVDEALEDVLRLSEGMTYKCAAADLDFGGGKAVIIGDPKKDKSSALFRAFGQYVDSLNGRFYTGTDMGTTMDDFVQATKETNFINGIPEAYGGSGDSSVPTAQGVIYGLKATNQFLFGNETLADRTYAVQGLGKVGFKLTEQLLQAGAHVIVTDIHEEAITAIEARAAELGAPLTTVSAEEIYQSDADIFVPCALGGILNDHTISQLRVKAVVGSANNQLQDRRHAKDLHDKGILYAPDYIVNAGGLIQVADELYGPNKERVLLKTKTIYRTLLEIYEQANLDVITTLEAADRKCQKRLEQQRHRNNFYTRTRSPKWEVKE

SEQ ID NO: 6 - protein, 376 aa, Salinicoccus roseus
MEYSRTETKDMFEKIAGHEQVLFCNDPATGLQAIIAIHDTTLGPALGGTRMYPYESVDAALDDVLRLSEGMTHKCAASGLDFGGGKAVIIADPATDKSPALFRSFGQFVDSLNGRFYTGTDMGTTTEDFVEAFKETHFINGIPEEYGGSGDSSIPTAQGVLYALEATNEHLFNSRDLGKRSYAIQGLGKVGYKVAEQLLEAGAKLFVTDINEAAILSIENYASGTTGELKIVESDEIYGVEADIFVPCAMGGIINDETLKQLQVKAVVGSANNQLSDNSIAEKLQQSGILYAPDFIVNAGGLIQVADELYGPNPKRVLAKTKTIYQSLLKIYQQAEMDATTTTDAANQKVTHVLEEQKHRNNFFSRKRRPKWEVRE

SEQ ID NO: 7 - protein, 379 aa, Priestia abyssalis
MNVMKQPVQMQETVLEKMHEHEQIVFCNDPATGLKAIIAIHNTTLGPALGGCRMHPYRTMDEALEDVLRLSKGMTYKCAAADVDFGGGKSVIIGDPTKDKTPAMFRAFGQFVDSLNGRFYTGTDMGTSTDDFIHASKETNCIVGIPEAYGGGGDSSVPTALGVIYGIKATNKVAFGTDDLSGKAYAIQGLGKVGFKVAEQLLESGGDLYVTDINEKAIEAIQERAKQLGGNVRVVNGNDIYSADADVFVPCAFGGIINDDTLDMLKVKAIAGSANNQLLTEKHGEILKNKGILYAPDYIVNGGGLIQVADELYGYNKERVLTKTKSIYTSLLEVYEQSDLDDITTIQAANRLCEKRMEDRKKQNSFFSHTKRPKWDIRK

SEQ ID NO: 8 - protein, 379 aa, Weizmannia coagulans
MNVIKPHMQTTETLLDKMQEHEQIVFCNDPATGLKAIIAIHNTTLGPALGGCRMQPYRTLDEALEDVLRLSKGMTYKCAAADVDFGGGKAVLIGDPSKDKTPAMFRAFGQFVDSLNGRFYTGTDMGTSMDDFIHAAKETRCIVGIPECYGGGGDSSIPTALGVIYGIKAVNKAVFGSADLAGKAYAVQGLGKVGFKVAEHLLEAGADLYVSDIHEEAVQAILARAKQLGSAVRVVKSHEIYSAEADVFVPCALGGILNDDTIHQLKVKAVAGSANNQLLSEKHSEALKRKGILYAPDYIVNAGGLIQVADELYGYQKERVYTKTKSIYTSLIEVFKQAEMDNITTSQAANRLCEKRIEERKKQNSFFTHSKRPKWDIRK

SEQ ID NO: 9 - DNA, 1140 bp, Sporosarcina ureae
  CDS (1)..(1140): amino acid dehydrogenase
atgattttggtaactttagaacagactttacaagacgacaaggcaagtgtattggataaaatggtcgagcatgaacaaattctattttgtcatgataaagcaaccggtcttcaagccatcattgcactccacgatacgacgatgggacctgcacttggtggatgtcgcatggcgccgtataaaacgatggatctcgcattacaagatgttcttcgcctttctaaagggatgacctataaatgcgcggcagctgatgtagactttggtggcggaaaatccgtcatcatcggagatccgttaaaagataaaacgcctgagaaattccgtgctttcgggcaattcatcgaatcactgaacggacgcttctatacaggaacagacatgggcacaacgcttgaagactttgtgcaagctatgaaagaaacaaactacatcgtgggcaagccggttgaatatggcggcggtggagactcatccatccctactgcactcggagtcttctatggtattaaagcgacaaaccagaatgtgtttggtgacgacaaagtagaaggtcgaacatacagtattcaaggtcttggaaaagtaggttacaaagtagcggaacatattatcaatgaaggcggaaacgtgatcgtcacagatattcatgagcaagcgattgcagatatacagaagctcggtggaagcgctgtcaaggtcgtctcaagtgatgagatttacagccagcaagcagatatttttgttccttgtgcatttggtggcgtgatcaatgacgacacgctcaacgtactgaaagtccgaggaatctcgggctccgcaaacaatcagctcgcagaaagtcgtcacggagaactgcttcgtgaaaaaggtattctgtacgcacccgactatatcgttaacggtggcggactgatccaagtggcggatgaactgtacggaacgaatcctgcacgtgtactcgctaaaactgaaaacatctacaactcactgcttgaagtattccatcaggcggaacaggatcatattaccactgctactgctgcagaccgtatgtgcgaaaagcgtattgcggatgccaagaaccgcaacagcttcttcacgcagtcaaaccgaccgaaatggaattttcatcagtaa

SEQ ID NO: 10 - protein, 379 aa, Sporosarcina ureae
MILVTLEQTLQDDKASVLDKMVEHEQILFCHDKATGLQAIIALHDTTMGPALGGCRMAPYKTMDLALQDVLRLSKGMTYKCAAADVDFGGGKSVIIGDPLKDKTPEKFRAFGQFIESLNGRFYTGTDMGTTLEDFVQAMKETNYIVGKPVEYGGGGDSSIPTALGVFYGIKATNQNVFGDDKVEGRTYSIQGLGKVGYKVAEHIINEGGNVIVTDIHEQAIADIQKLGGSAVKVVSSDEIYSQQADIFVPCAFGGVINDDTLNVLKVRGISGSANNQLAESRHGELLREKGILYAPDYIVNGGGLIQVADELYGTNPARVLAKTENIYNSLLEVFHQAEQDHITTATAADRMCEKRIADAKNRNSFFTQSNRPKWNFHQ

SEQ ID NO: 11 - DNA, 1140 bp, Sporosarcina ureae R-04
  CDS (1)..(1140)
atgattttggtaactttagaacagactttacaagacgacaaggcaagtgttttggataaaatggtcgagcatgaacaaattctattttgtcatgataaagcaaccggtcttcaagccatcattgcagtccacgatacgactatgggacctgcactcggtggatgtcgcatggcgccttataaaacgatggatctcgcattaaaagatgttcttcgcctttcaaaagggatgacatataaatgtgcggcagctgatgtagactttggcggcggaaaatccgtcatcatcggagacccgctaaaagataaaacgcctgagaaattccgtgctttcggtcaattcatcgaatcattgaacggacgcttctatacaggtacagacatgggcacaacgcttgaagactttgtgcatgccatgaaagaaacaaactacatcgtgggcaagccggtcgaatatggtggcggtggagactcatcgatccctactgcactcggagtcttctatggcattaaagcgacaaaccagaatctgtttggcgacgacaaagtagaaggccgaaaatacagtatccaaggtcttgggaaagtaggttacaaagtagctgaacatattatcaacgaaggtggaaacgtgatcgtcacagatattaatgagcaagcgattgcagatattcagaagctcggtggaagcgctgtcagggtcgtatcaagtgaggagatttacagtcagcaagcagatgtttttgttccttgtgcatttggtggcgtgatcaatgacgacacgctaaaggtgctgaaagtacgaggaatctccggttcagcaaacaatcagctcgcggaaagccgccatggagagctactacgtgaaaagggtattttgtacgcaccagactatatcgtcaacggcggcggtttaatccaagtggcggatgaattgtacggaacgaatcctgcacgtgtactcgctaaaactgaaaacatctatacctcactgcttgaagtattccatcaggcagaacaggatcatatgacaactgccactgccgcagaccgtatgtgtgaaaagcgtattgcggatgccaagaatcgcaacagcttcttcacacagtcaaaccgaccgaaatggaattttcatcagtaa

SEQ ID NO: 12 - protein, 379 aa, Sporosarcina ureae R-04
MILVTLEQTLQDDKASVLDKMVEHEQILFCHDKATGLQAIIAVHDTTMGPALGGCRMAPYKTMDLALKDVLRLSKGMTYKCAAADVDFGGGKSVIIGDPLKDKTPEKFRAFGQFIESLNGRFYTGTDMGTTLEDFVHAMKETNYIVGKPVEYGGGGDSSIPTALGVFYGIKATNQNLFGDDKVEGRKYSIQGLGKVGYKVAEHIINEGGNVIVTDINEQAIADIQKLGGSAVRVVSSEEIYSQQADVFVPCAFGGVINDDTLKVLKVRGISGSANNQLAESRHGELLREKGILYAPDYIVNGGGLIQVADELYGTNPARVLAKTENIYTSLLEVFHQAEQDHMTTATAADRMCEKRIADAKNRNSFFTQSNRPKWNFHQ

SEQ ID NO: 13 - protein, 379 aa, Sporosarcina sp. P3
MNLVTLEQTLQDDKASVLDKMVEHEQVLFCNDKATGLQAIIAVHDTTMGPALGGCRMAPYQTMDLALKDVLRLSKGMTYKCAAADVDFGGGKSVIIGDPLKDKTPEKFRAFGQFIESLNGRFYTGTDMGTTLEDFVQAMKETNYIVGKPVEYGGGGDSSIPTALGVFYGIKATNQNLFGDDKVEGRKYSIQGLGKVGYKVAEHIINEGGNVIVTDINEQAIADIQQLGGNAVKVVSSEEIYSQQADIFVPCAFGGVINDDTLNVLKVRGISGSANNQLAESRHGELLREKGILYAPDYIVNGGGLIQVADELYGTNPARVLAKTENIYNSLLEVFNQAEQDHIPTATAADRMCEKRIADAKNRNSFFTQSNRPKWNFHQ

SEQ ID NO: 14 - protein, 378 aa, Bacillus sp. OxB-1
MVTLEQSLKNENASVLDKMVEHEQVLFCNDKATGLQAIIAIHDTTMGPALGGCRMAPYKTMDDALKDVLRLSKGMTYKCAAADVDFGGGKSVIIGDPLKDKTPEKFRAFGQFIDSLNGRFYTGTDMGTTLDDFVQAMKETNYIAGKPVEFGGGGDSSIPTALGVVYGVKATSKALFGDDKLEGKTFTIQGLGKVGYKVAEHILNEGGNIIVTDINEKALEDIQKLGGSSVKVVASDDIYSQDADIFIPCAFGGIINDDTIDVLKVRGISGSANNQLSESRHGDVLRERGILYAPDYIVNGGGLIQVADELYGANPARVLAKTENIYNSLLEVFTQAEQDQITTATAADRMCEKRIADAKNRNSFFSQAKRPKWHFQHQ

SEQ ID NO: 15 - protein, 379 aa, Bacillus sp. FJAT-14578
MNVIKQPVQMQETVLEKMHEHEQIVFCNDPATGLKAIIAIHNTTLGPALGGCRMHPYRTMDEALEDVLRLSKGMTYKCAAADVDFGGGKSVIIGDPSKDKTPAMFRAFGQFVDSLNGRFYTGTDMGTSTDDFIHASKETNCIVGIPEAYGGGGDSSVPTALGVIYGIQATNKVAFGTDDLSGKTYAIQGLGKVGFKVAEQILESGGDLYVTDINEKAIEAIQERAKQLGGNVRVVSGNDIYSADADVFVPCAFGGIINDDTLDMLKVRAIAGSANNQLLTEKHGELLKNKGILYAPDYIVNGGGLIQVADELYGYNKERVLTKTKSIYTSLLEVYQQSELDDITTIQAANRLCEKRMEDRKKQNSFFSHTTRPKWAIRK

SEQ ID NO: 16 - protein, 380 aa, Paenisporosarcina sp. TG-14
MKITEKLSITRDFELFDKMSEHEQVVFCNDPSTGLRAIIAIHNTTLGPALGGCRMQPYTSVDEALEDVLRLSKGMTYKCAAADVDFGGGKAVIIGDPRTDKTPELFRAFGQFVNSLGGRFFTGTDMGTNMDDFIHAMKETNFVNGLPEAYGGGGDSSIPTAAGVLYGIKATNQVLYGKDDLGGLVYAIQGLGKVGFKIASSLLEEGAHLIVSDINEDSLKAIEEIATKTSGTVKIVAKEDIYSQQADMFVPCAFGGIINDNTIAQFQVKAIAGSANNQLLHESHGKMLQEKGILYAPDYIVNSGGLIQVADELYGTNHERVLAKTKHIYDAILEVFKQAEISGLSTMESANKMCEKKIEVRGKRNGFYTASVKPKWSFRN

SEQ ID NO: 17 - DNA, 1065 bp, Rhodococcus jostii
  CDS (1)..(1065): Glu/Leu/Phe/Val dehydrogenase
atgagactcgacaagacattgtgctgggacggcgaactgacggtgacgcggcacgaccgggaaaccggagtgtccttcatcatccgcttggactcgacccgactcggacctgccgcgggtggcacccgcgcggcacagtacgcatccttcggtgaggcgctcgaagatgccggcagactcgcgggtgcgatgacgttgaaaatggccgtcagcaacctcccgatgggcggtggcaagtcggtcattgctctccctgctccacgcaatgagatcgaccagggcacatgggaacgaatcctgaccctgcacgccgagaacatcgacaaactcgccggcaactactggacaggccccgacgtcaataccaactcgtcggacatggacattctcaacgacacaaccgagttcgtcttcggccgttccgccgcgcgtggcggagccggttccagtgcactcaacacgtcgttgggtgtcttcgaggccatgaaggcgactgccatgcaccgaggactcgggacgctcgacggtctcaccgtgctcgttcagggcctcggcgcggtcggcagcggggtggccgtcctcgcgtcgcaggccggggcgcgcctcctcgtctcggataccgactccgagcggttggcctgggctcggcaactcggtcatgccgttgtcgggatcgatgacgtcctgaccacgccctgtgacatcttcgcgccctgcgcgatgggcggcgtactcgacagcgcggtcgcctgtgaactgcccgcgtccgcagtggtcggcgccgcgaacaacgtccttgccgacgacgaggcgggagaaatcctccacaagcgcgatgtcctgtacgcgcccgacttcgttaccaacgccggtggcgcacttcacttggtcggccgagaggtcctcggctggagcgaggagacggtcggagcgcgcacccgacgcatcggagaaacacttgccgaggtctactctctcgccgagagcgaggacatcgacactgatcgtgccgcgcgaattctggctcgccgccggatcgagacagcgactccgctccgcgtatag

SEQ ID NO: 18 - protein, 354 aa, Rhodococcus jostii
MRLDKTLCWDGELTVTRHDRETGVSFIIRLDSTRLGPAAGGTRAAQYASFGEALEDAGRLAGAMTLKMAVSNLPMGGGKSVIALPAPRNEIDQGTWERILTLHAENIDKLAGNYWTGPDVNTNSSDMDILNDTTEFVFGRSAARGGAGSSALNTSLGVFEAMKATAMHRGLGTLDGLTVLVQGLGAVGSGVAVLASQAGARLLVSDTDSERLAWARQLGHAVVGIDDVLTTPCDIFAPCAMGGVLDSAVACELPASAVVGAANNVLADDEAGEILHKRDVLYAPDFVTNAGGALHLVGREVLGWSEETVGARTRRIGETLAEVYSLAESEDIDTDRAARILARRRIETATPLRV

SEQ ID NO: 19 - protein, 368 aa, Rhodococcus opacus
MRLDKTLCWDGELTVTRHDRETGVSFIIRLDSTRLGPAAGGTRAAQYASFGEALDDAGRLAGAMTVKMAVSNLPMGGGKSVIALPAPRSEIDQDTWKRILTLHAENIDKLAGNYWTGPDVNTNSSDMDIINDTTEFVFGRSAARGGAGSSAFNTSLGVFEAMKATAVHRGLGTLDGLTVLVQGLGAVGSGVADLASQAGARLLVADTDSERLAWARQLGRAIVGIDDVLTTPCDIFAPCAMGGVLDSAVACELPASAVVGAANNVLADDEAAEILHKRDVTYAPDFVTNAGGAFHLVGREVLGWSEETVGARTRGIGDTLAEVYSLAESEDIDTDRAARILARRRIETADSAPRRANSFPEGSEMPVL

SEQ ID NO: 20 - protein, 355 aa, Nocardia araoensis
MSLHDTLRWQGELTATRHDPQTGASFVIRISSTSLGPAAGGTRAAQYASFADALADAGKLADAMTLKMAVSNLPMGGGKSVIALPAPRKDIDQATWNRILTLHAENIDKLKGNYWTGPDVNTNSADMDTLSETTKFVFGRSPERGGAGSSAFNTSLGVLAAMKATAAYRGLGTLDGLRVLIQGLGAVGSGVARLAADAGARLLVSDTDPARMAWAQQRGYTVVGVDEILRTPCDIFAPCAMGAVIGTDVAAELPASAVVGAANNILADDHAGAILRERGILYAPDFVANAGGAFHLVGREVLGWGRETVEEHTRGIGDILTEVYTLAESSGITTDLAARAVARTRLETSRAETAA

SEQ ID NO: 21 - protein, 354 aa, Mycobacterium kyorinense
MSLDDVLRWDGELTVTRHDPETGASFVIRVDSTRLGQAAGGTRAAQYPSLAGALEDAGKLAGAMTLKMAVSGLPMGGGKSVIALPAPRKDIAPERWQRILQLHAENIDRLNGTYWTGPDVNTDSADMDVLNETTEFVFGRSADRGGAGSSAVNTALGVFEAMKATASHAGLGSLDGLSILIQGLGAVGGRLATLAAEAGARLLVSDTDFQRLSWAREFGCTIVEPDAVSTTPCDIFAPCAVGGVIDSATAARLTTSAIVGAANNILADERAGDILRQRGILYAPDFVANAGGAFHLVGREVLGWDDDTVAQHTNRIGDTLIEVYAISEAEDITTDAAARILARRRAAHSQPVGG

SEQ ID NO: 22 - protein, 368 aa, Rhodococcus
MSIDDELRWDGELTVTRHDRETGTTFVIRIDSTRLGPASGGTRAAHYPSIGHALADAGKLAGAMTLKMAVSDLPMGGGKSVIALPAPRNRIDSATWSRILDIHAENIDKLEGNYWTGPDVNTNSSDMDQLSRTTKYVFGRSVDNGGAGSSAHATALGVFEAMKATARRRGLGTLDGRKVLVQGLGAVGGDVVRLAAQAGARLLVADTDPQRLEAASLAGHTIVPADEVLRTSCDLFAPCAMGGIIDSAAAASIPTLAVAGAANNILTDAAAGEVLRSRGVLCAPDFVANAGGAFHLVGREVLGWNEDDVVERTRGIGRTLDEVYSLSEERGITTEAAALLLARTRLSPASTTAAATSATPAAAAASPV

SEQ ID NO: 23 - DNA, 1071 bp, Rhodococcus sp.
  CDS (1)..(1071)
atgagtatcgacagcgcactgaactgggacggggaaatgacggtcacccgattcgaccgggagactggtgcccatttcgtcattcgactcgattcgacccaactcggaccggcggccggaggcaccagagccgcacagtactcacagctggcggacgccctcaccgacgccggcaaattggcgggggcgatgacgttgaagatggcagtgagcaaccttccgatgggcgggggcaaatccgtcattgcgcttcctgcgccgcgtcattcgatcgatccgagcacgtgggcacgcatcctccgaatccacgccgagaacatcgacaagttgtccggcaactactggaccggaccggacgtcaacaccaattcggcagacatggatactctgaacgacaccaccgagttcgtgttcggacggtcgctcgaacgcggcggcgcgggttcgagcgcgttcaccaccgccgttggcgtgttcgaggcgatgaaggcgaccgtcgcgcaccgtgggctgggctcactcgacggtttgacggtcctggtccaaggactgggggcagtcggaggatcattggcatccctggccgccgaagcgggtgcgcaactcctggtggcagacaccgacaccgagcgagtagcgcacgctgttgcgttgggccacacagcggttgccctcgaggacgttctgtccaccccgtgtgatgtcttcgcaccctgcgcaatgggcggcgtcatcaccaccgaggtggcgcgaacactcgactgttccgtcgtggccggtgccgccaacaacgtcatcgccgacgaggccgcctcggacatcctgcacgcacgcggaattctgtacgctcccgacttcgtggccaacgccggcggtgccatccacctcgtaggccgggaggttctcggttggtccgagtcggttgtccacgaacgagcagttgccataggcgacaccctgaatcaggtcttcgagatctccgacaacgacggcgtcaccccggacgaggccgcccgcactctcgctggacggcgcgcccgcgaggcctcgacaacgacagcgactgcctag

SEQ ID NO: 24 - protein, 356 aa, Rhodococcus sp.
MSIDSALNWDGEMTVTRFDRETGAHFVIRLDSTQLGPAAGGTRAAQYSQLADALTDAGKLAGAMTLKMAVSNLPMGGGKSVIALPAPRHSIDPSTWARILRIHAENIDKLSGNYWTGPDVNTNSADMDTLNDTTEFVFGRSLERGGAGSSAFTTAVGVFEAMKATVAHRGLGSLDGLTVLVQGLGAVGGSLASLAAEAGAQLLVADTDTERVAHAVALGHTAVALEDVLSTPCDVFAPCAMGGVITTEVARTLDCSVVAGAANNVIADEAASDILHARGILYAPDFVANAGGAIHLVGREVLGWSESVVHERAVAIGDTLNQVFEISDNDGVTPDEAARTLAGRRAREASTTTATA

SEQ ID NO: 25 - DNA, 1095 bp, Lysinibacillus sphaericus
  CDS (1)..(1095): Glu/Leu/Phe/Val dehydrogenase
atggaaatcttcaagtacatggagaagtacgattacgaacagttggtcttttgccaggatgaagcatcaggcctcaaggctatcatcgcgatccacgatactactctgggaccagctcttggaggtgcacgcatgtggacttatgcgacggaggagaacgctatcgaagatgccctgcgcttggcacgtggtatgacctataagaacgctgccgctggcctgaacctcggcggtggtaagaccgtcatcattggtgatcccttcaaggacaagaacgaggagatgttccgtgcattgggtcggttcattcagggccttaacggccgatacatcaccgccgaagatgtgggcacaaccgtaacggatatggacctgatccacgaagaaaccaactacgtgacgggaatttccccagcattcggttcctctgggaatccgtccccagttaccgcgtatggtgtctatcgaggcatgaaagctgcggcaaaggaagcgtttggaaccgacatgctggaagggcgcaccatctccgttcaagggctgggcaatgtcgcatacaaactgtgcgaatacctccataacgagggtgctaaactggtcgttaccgacatcaaccaagccgctattgatcgcgtggtcaacgacttcggagccacagcagttgcaccagacgaaatctactcccaggaggtggacatcttcagcccgtgtgcccttggagcgatccttaacgacgaaaccattcctcagttgaaagccaaggtgattgcagggtcagccaacaatcagctccaagactctcggcatggcgattatctccacgaattgggcattgtatatgctcctgactacgtcatcaacgctggtggcgtgatcaacgttgccgacgagctttacggctacaatcgtgagcgcgcactcaaacgcgttgatggcatttacgattcgatcgagaagatctttgagattagcaagcgcgattctatccccacttacgtagcggcaaatcgcctggctgaggaacgcattgctcgtgtggcgaaatcgcgttcccagttcctgaagaacgagaagaacatcctgaatggccgctaa

SEQ ID NO: 26 - protein, 364 aa, Lysinibacillus sphaericus
MEIFKYMEKYDYEQLVFCQDEASGLKAIIAIHDTTLGPALGGARMWTYATEENAIEDALRLARGMTYKNAAAGLNLGGGKTVIIGDPFKDKNEEMFRALGRFIQGLNGRYITAEDVGTTVTDMDLIHEETNYVTGISPAFGSSGNPSPVTAYGVYRGMKAAAKEAFGTDMLEGRTISVQGLGNVAYKLCEYLHNEGAKLVVTDINQAAIDRVVNDFGATAVAPDEIYSQEVDIFSPCALGAILNDETIPQLKAKVIAGSANNQLQDSRHGDYLHELGIVYAPDYVINAGGVINVADELYGYNRERALKRVDGIYDSIEKIFEISKRDSIPTYVAANRLAEERIARVAKSRSQFLKNEKNILNGR

SEQ ID NO: 27 - protein, 364 aa, Bacillus velezensis
MEIFKYMEKYDYEQLVFCQDEASGLKAIIAIHDTTLGPALGGARMWTYATEENAIEDALRLARGMTYKNAAAGLNLGGGKTVIIGDPFKDKNEEMFRALGRFIQGLNGRYITAEDVGTTVTDMDLIHEETNYVTGISPAFGSSGNPSPVTAYGVYRGMKAAAKEAFGTDSLEGRTISVQGLGNVAYKLCEYLHNEGAKLVVTDINQAAIDRVVNDFGARAVAPEEIYSQEVDIFSPCALGAILNDETIPQLKAKVIAGSANNQLQDSRHGDYLHELGIVYAPDYVINAGGVINVADELYGYNRERAMKRVDGIYDSIEKIFDISKRDGIPTYVAANRLAEERIARVSKSRSQFLKNEKNILNGR

SEQ ID NO: 28 - protein, 364 aa, Ureibacillus sp. Re31
MEIFKYLEKYDYEQVVFCQDKTSGLKAIIAIHDTTLGPALGGARMWSYASEEQAIEDALRLARGMTYKNAAAGLNLGGGKTVIIGDPFKDKNEEMFRALGRFIQGLNGRYITAEDVGTTVTDMDLIHEETDYVTGISPSFGSSGNPSPVTAYGVYLGMKAAAKEAFGTDSLQDRTIAVQGLGNVAYTLCEYLYNEGAKLVVTDINKNAVERVVNDFNAVAVDPGDIYSQDVDIFAPCALGAIINDETIPQLKAKVIAGSANNQLKDSAHGDLIHKMGIVYAPDYVINAGGVINVADELYGYNRERAMKRVEAIYDSISKIFEISKRDKIPTYLAANRLAEERIERISKSRSQFLKNGKNILNGR

SEQ ID NO: 29 - protein, 364 aa, Sporosarcina sp. EUR3 2.2.2
MEIFKYMETYDYEQLVFCQDKTSGLKAIIAIHDTTLGPALGGTRMWTYASEEEAIEDALRLAKGMTYKNAAAGLNLGGGKTVIIGDPLKDKNEEMFRAFGRFIQGLNGRYITAEDVGTTVADMDLIHEETNYVTGISPAFGSSGNPSPVTAYGVYRGMKAAAMEAFGSDSLEGKRIAVQGVGNVAYNLCRHLHEEGAILIVTDINKAAVDRVVEEFGATAVNTDEIYSQEVDIFAPCALGAIINDDTIPQLKAKVIAGAANNQLKETKHGDQIHELGIIYAPDYVINAGGVINVADELNGYNRERAMKRVETVYDNIAKVMEISRTQGIPTYVAADRLAEERIARVGKSRSQFLQNEKHILSGR

SEQ ID NO: 30 - protein, 364 aa, Viridibacillus
MEIFKYMEKYDYEQLVFCQDKTSGLKAIVAIHDTTLGPALGGTRMWTYASEEEAIEDALRLARGMTYKNAAAGLNLGGGKTVIIGDPLKDKNEEMFRALGRFVQGLNGRYITAEDVGTTVADMDLIHEETNYVTGISPAFGSSGNPSPVTAYGVFVGMKAAAKEAFGSEHLEGKKIAVQGLGNVAYTLCKYLHEEGAQLIVTDINESAVKRVVEDFGATAVDPTDIYAQDVDIFAPCALGAIINDETIPQLRAKVIAGSANNQLKDSIHGDIIHNEGIIYAPDYVINAGGVINVADELYGYNRDRAMKRVEGIYDNIEKVIDIAKRDGIPTYLAANRLAEERIERMAKSRSTFLQNGKHILTNR

SEQ ID NO: 31 - DNA, 1101 bp, Thermoactinomyces intermedius
  CDS (1)..(1101)
atgcgcgacgtgtttgaaatgatggaccgctatggccacgagcaggtcattttttgccgtcatccgcaaaccggtctcaaagcgatcatcgccttgcataatacaaccgcggggccggctttgggtggatgccgcatgatcccgtatgcttcgacggacgaagccttggaggatgttttgcggttgtccaaaggcatgacctataaatgcagtctggcggatgtggactttggcgggggaaaaatggttatcatcggcgatccgaaaaaagataaatcgccggagttgtttcgcgtgatcggccgttttgtgggcgggttaaacggccgtttctataccggaaccgacatgggaaccaatccggaagattttgtccatgccgccagggaatcgaaatcttttgccggattgccgaaatcgtacggcggaagcggggacacatccattcccaccgcgctcggggtgtttcacggaatgcgggccaccgcccggtttttatgggggacggatcagctgaaagggcgtgtggttgccatccaaggagtcggcaaggtgggagagcgcttgttgcagcttttggtcgaagtgggggcttactgcaaaattgccgacatcgattcggtgcgatgcgaacagctgaaagaaaagtatggcgacaaggtccaattggtggatgtgaaccggattcacaaggagagttgcgatattttctcgccttgcgccaaaggcggcgtggtcaatgatgacaccattgacgagttccgttgcctggccattgtcggatccgccaacaaccaactggtggaagaccggcatggggcactgcttcaaaaacggagcatttggtatgcacccgattatctggtgaatgccggcgggctgattcaagtggctgatgaactggaaggcttccatgaagagagagtgctcgccaaaaccgaagcgatttatgacatggtcctggatatttttcaccgggcgaaaaatgagaatattaccacttgtgaggcagcggaccggatcgtgatggagcgtttgaaaaagttaaccgatattcgccggatcttgttggaggatccccgcaacagcgcaaggaggtaa

SEQ ID NO: 32 - protein, 366 aa, Thermoactinomyces intermedius
MRDVFEMMDRYGHEQVIFCRHPQTGLKAIIALHNTTAGPALGGCRMIPYASTDEALEDVLRLSKGMTYKCSLADVDFGGGKMVIIGDPKKDKSPELFRVIGRFVGGLNGRFYTGTDMGTNPEDFVHAARESKSFAGLPKSYGGSGDTSIPTALGVFHGMRATARFLWGTDQLKGRVVAIQGVGKVGERLLQLLVEVGAYCKIADIDSVRCEQLKEKYGDKVQLVDVNRIHKESCDIFSPCAKGGVVNDDTIDEFRCLAIVGSANNQLVEDRHGALLQKRSIWYAPDYLVNAGGLIQVADELEGFHEERVLAKTEAIYDMVLDIFHRAKNENITTCEAADRIVMERLKKLTDIRRILLEDPRNSARR

SEQ ID NO: 33 - DNA, 1104 bp, Corynebacterium glutamicum ATCC 13032
  CDS (1)..(1104): branched-chain amino acid aminotransferase
atgacgtcattagagttcacagtaacccgtaccgaaaatccgacgtcacccgatcgtctgaaggaaattcttgccgcaccgaagttcggtaagttcttcaccgaccacatggtgaccattgactggaacgagtcggaaggctggcacaacgcccaattagtgccatacgcgccgattcctatggatcctgccaccaccgtattccactacggacaggcaatttttgagggaattaaggcctaccgccattcggacgaaaccatcaagactttccgtcctgatgaaaacgccgagcgtatgcagcgttcagcagctcgaatggcaatgccacagttgccaaccgaggactttattaaagcacttgaactgctggtagacgcggatcaggattgggttcctgagtacggcggagaagcttccctctacctgcgcccattcatgatctccaccgaaattggcttgggtgtcagcccagctgatgcctacaagttcctggtcatcgcatccccagtcggcgcttacttcaccggtggaatcaagcctgtttccgtctggctgagcgaagattacgtccgcgctgcacccggcggaactggtgacgccaaatttgctggcaactacgcggcttctttgcttgcccagtcccaggctgcggaaaagggctgtgaccaggtcgtatggttggatgccatcgagcacaagtacatcgaagaaatgggtggcatgaaccttgggttcatctaccgcaacggcgaccaagtcaagctagtcacccctgaactttccggctcactacttccaggcatcacccgcaagtcacttctacaagtagcacgcgacttgggatacgaagtagaagagcgaaagatcaccaccaccgagtgggaagaagacgcaaagtctggcgccatgaccgaggcatttgcttgcggtactgcagctgttatcacccctgttggcaccgtgaaatcagctcacggcaccttcgaagtgaacaacaatgaagtcggagaaatcacgatgaagcttcgtgaaaccctcaccggaattcagcaaggaaacgttgaagaccaaaacggatggctttacccactggttggctaa

SEQ ID NO: 34 - protein, 367 aa, Corynebacterium glutamicum ATCC 13032
MTSLEFTVTRTENPTSPDRLKEILAAPKFGKFFTDHMVTIDWNESEGWHNAQLVPYAPIPMDPATTVFHYGQAIFEGIKAYRHSDETIKTFRPDENAERMQRSAARMAMPQLPTEDFIKALELLVDADQDWVPEYGGEASLYLRPFMISTEIGLGVSPADAYKFLVIASPVGAYFTGGIKPVSVWLSEDYVRAAPGGTGDAKFAGNYAASLLAQSQAAEKGCDQVVWLDAIEHKYIEEMGGMNLGFIYRNGDQVKLVTPELSGSLLPGITRKSLLQVARDLGYEVEERKITTTEWEEDAKSGAMTEAFACGTAAVITPVGTVKSAHGTFEVNNNEVGEITMKLRETLTGIQQGNVEDQNGWLYPLVG

SEQ ID NO: 35 - DNA, 1851 bp, Corynebacterium glutamicum ATCC 13032
  CDS (1)..(1851): leuA (2-isopropylmalate synthase)
atgtctcctaacgatgcattcatctccgcacctgccaagatcgaaaccccagttgggcctcgcaacgaaggccagccagcatggaataagcagcgtggctcctcaatgccagttaaccgctacatgcctttcgaggttgaggtagaagatatttctctgccggaccgcacttggccagataaaaaaatcaccgttgcacctcagtggtgtgctgttgacctgcgtgacggcaaccaggctctgattgatccgatgtctcctgagcgtaagcgccgcatgtttgagctgctggttcagatgggcttcaaagaaatcgaggtcggtttcccttcagcttcccagactgattttgatttcgttcgtgagatcatcgaaaagggcatgatccctgacgatgtcaccattcaggttctggttcaggctcgtgagcacctgattcgccgtacttttgaagcttgcgaaggcgcaaaaaacgttatcgtgcacttctacaactccacctccatcctgcagcgcaacgtggtgttccgcatggacaaggtgcaggtgaagaagctggctaccgatgccgctgaactaatcaagaccatcgctcaggattacccagacaccaactggcgctggcagtactcccctgagtccttcaccggcactgaggttgagtacgccaaggaagttgtggacgcagttgttgaggtcatggatccaactcctgagaacccaatgatcatcaacctgccttccaccgttgagatgatcacccctaacgtttacgcagactccattgaatggatgcaccgcaatctaaaccgtcgtgattccattatcctgtccctgcacccgcacaatgaccgtggcaccggcgttggcgcagctgagctgggctacatggctggcgctgaccgcatcgaaggctgcctgttcggcaacggcgagcgcaccggcaacgtctgcctggtcaccctggcactgaacatgctgacccagggcgttgaccctcagctggacttcaccgatatacgccagatccgcagcaccgttgaatactgcaaccagctgcgcgttcctgagcgccacccatacggcggtgacctggtcttcaccgctttctccggttcccaccaggacgctgtgaacaagggtctggacgccatggctgccaaggttcagccaggtgctagctccactgaagtttcttgggagcagctgcgcgacaccgaatgggaggttccttacctgcctatcgatccaaaggatgtcggtcgcgactacgaggctgttatccgcgtgaactcccagtccggcaagggcggcgttgcttacatcatgaagaccgatcacggtctgcagatccctcgctccatgcaggttgagttctccaccgttgtccagaacgtcaccgacgctgagggcggcgaggtcaactccaaggcaatgtgggatatcttcgccaccgagtacctggagcgcaccgcaccagttgagcagatcgcgctgcgcgtcgagaacgctcagaccgaaaacgaggatgcatccatcaccgccgagctcatccacaacggcaaggacgtcaccgtcgatggccgcggcaacggcccactggccgcttacgccaacgcgctggagaagctgggcatcgacgttgagatccaggaatacaaccagcacgcccgcacctcgggcgacgatgcagaagcagccgcctacgtgctggctgaggtcaacggccgcaaggtctggggcgtcggcatcgctggctccatcacctacgcttcgctgaaggcagtgacctccgccgtaaaccgcgcgctggacgtcaaccacgaggcagtcctggctggcggcgtttaa

SEQ ID NO: 36 - protein, 616 aa, Corynebacterium glutamicum ATCC 13032
MSPNDAFISAPAKIETPVGPRNEGQPAWNKQRGSSMPVNRYMPFEVEVEDISLPDRTWPDKKITVAPQWCAVDLRDGNQALIDPMSPERKRRMFELLVQMGFKEIEVGFPSASQTDFDFVREIIEKGMIPDDVTIQVLVQAREHLIRRTFEACEGAKNVIVHFYNSTSILQRNVVFRMDKVQVKKLATDAAELIKTIAQDYPDTNWRWQYSPESFTGTEVEYAKEVVDAVVEVMDPTPENPMIINLPSTVEMITPNVYADSIEWMHRNLNRRDSIILSLHPHNDRGTGVGAAELGYMAGADRIEGCLFGNGERTGNVCLVTLALNMLTQGVDPQLDFTDIRQIRSTVEYCNQLRVPERHPYGGDLVFTAFSGSHQDAVNKGLDAMAAKVQPGASSTEVSWEQLRDTEWEVPYLPIDPKDVGRDYEAVIRVNSQSGKGGVAYIMKTDHGLQIPRSMQVEFSTVVQNVTDAEGGEVNSKAMWDIFATEYLERTAPVEQIALRVENAQTENEDASITAELIHNGKDVTVDGRGNGPLAAYANALEKLGIDVEIQEYNQHARTSGDDAEAAAYVLAEVNGRKVWGVGIAGSITYASLKAVTSAVNRALDVNHEAVLAGGV

SEQ ID NO: 37 - DNA, 1881 bp, Corynebacterium glutamicum ATCC 13032
  gene (1)..(1881): ilvB (acetolactate synthase I/II/III large subunit CDS)
gtgaatgtggcagcttctcaacagcccactcccgccacggttgcaagccgtggtcgatccgccgcccctgagcggatgacaggtgcaaaggcaattgttcgatcgctcgaggagcttaacgccgacatcgtgttcggtattcctggtggtgcggtgctaccggtgtatgacccgctctattcctccacaaaggtgcgccacgtcttggtgcgccacgagcagggcgcaggccacgcagcaaccggctacgcgcaggttactggacgcgttggcgtctgcattgcaacctctggcccaggagcaaccaacttggttaccccaatcgctgatgcaaacttggactccgttcccatggttgccatcaccggccaggtcggaagtggcctgctgggtaccgacgctttccaggaagccgatatccgcggcatcaccatgccagtgaccaagcacaacttcatggtcaccaaccctaacgacattccacaggcattggctgaggcattccacctcgcgattactggtcgccctggccctgttctggtggatattcctaaggatgtccagaacgctgaattggatttcgtctggccaccaaagatcgacctgccaggctaccgcccagtttcaacaccacatgctcgccagatcgagcaggcagtcaagctgatcggtgaggccaagaagcccgtcctttacgttggtggtggcgtaatcaaggctgacgcacacgaagagcttcgtgcgttcgctgagtacaccggcatcccagttgtcaccaccttgatggctttgggtactttcccagagtctcacgagctgcacatgggtatgccaggcatgcatggcactgtgtccgctgttggtgcactgcagcgcagcgacctgctgattgctatcggctcccgctttgatgaccgcgtcaccggtgacgttgacaccttcgcgcctgacgccaagatcattcacgccgacattgatcctgccgaaatcggcaagatcaagcaggttgaggttccaatcgtgggcgatgcccgcgaagttcttgctcgtctgctggaaaccaccaaggcaagcaaggcagagaccgaggacatctccgagtgggttgactacctcaagggcctcaaggcacgtttcccgcgtggctacgacgagcagccaggcgatctgctggcaccacagtttgtcattgaaaccctgtccaaggaagttggccccgacgcaatttactgcgccggcgttggccagcaccaaatgtgggcagctcagttcgttgactttgaaaagccacgcacctggctcaactccggtggactgggcaccatgggctacgcagttcctgcggcccttggagcaaaggctggcgcacctgacaaggaagtctgggctatcgacggcgacggctgtttccagatgaccaaccaggaactcaccaccgccgcagttgaaggtttccccattaagatcgcactaatcaacaacggaaacctgggcatggttcgccaatggcagaccctattctatgaaggacggtactcaaatactaaacttcgtaaccagggcgagtacatgcccgactttgttaccctttctgagggacttggctgtgttgccatccgcgtcaccaaagcggaggaagtactgccagccatccaaaaggctcgagagatcaacgaccgcccagtagtcatcgacttcatcgtcggtgaagacgcacaggtatggccaatggtgtctgctggatcatccaactccgatatccagtacgcactcggattgcgcccattctttgatggtgatgaatctgcagcagaagatcctgccgacattcacgaagccgtcagcgacattgatgccgccgttgaatcgaccgaggcataa

SEQ ID NO: 38 - protein, 626 aa, Corynebacterium glutamicum ATCC 13032
MNVAASQQPTPATVASRGRSAAPERMTGAKAIVRSLEELNADIVFGIPGGAVLPVYDPLYSSTKVRHVLVRHEQGAGHAATGYAQVTGRVGVCIATSGPGATNLVTPIADANLDSVPMVAITGQVGSGLLGTDAFQEADIRGITMPVTKHNFMVTNPNDIPQALAEAFHLAITGRPGPVLVDIPKDVQNAELDFVWPPKIDLPGYRPVSTPHARQIEQAVKLIGEAKKPVLYVGGGVIKADAHEELRAFAEYTGIPVVTTLMALGTFPESHELHMGMPGMHGTVSAVGALQRSDLLIAIGSRFDDRVTGDVDTFAPDAKIIHADIDPAEIGKIKQVEVPIVGDAREVLARLLETTKASKAETEDISEWVDYLKGLKARFPRGYDEQPGDLLAPQFVIETLSKEVGPDAIYCAGVGQHQMWAAQFVDFEKPRTWLNSGGLGTMGYAVPAALGAKAGAPDKEVWAIDGDGCFQMTNQELTTAAVEGFPIKIALINNGNLGMVRQWQTLFYEGRYSNTKLRNQGEYMPDFVTLSEGLGCVAIRVTKAEEVLPAIQKAREINDRPVVIDFIVGEDAQVWPMVSAGSSNSDIQYALGLRPFFDGDESAAEDPADIHEAVSDIDAAVESTEA

SEQ ID NO: 39 - DNA, 519 bp, Corynebacterium glutamicum ATCC 13032
  CDS (1)..(519): ilvN (acetolactate synthase I/III small subunit)
atggctaattctgacgtcacccgccacatcctgtccgtactcgttcaggacgtagacggaatcatttcccgcgtatcaggtatgttcacccgacgcgcattcaacctcgtgtccctcgtgtctgcaaagaccgaaacacacggcatcaaccgcatcacggttgttgtcgacgccgacgagctcaacattgagcagatcaccaagcagctcaacaagctgatccccgtgctcaaagtcgtgcgacttgatgaagagaccactatcgcccgcgcaatcatgctggttaaggtctctgcggacagcaccaaccgtccgcagatcgtcgacgccgcgaacatcttccgcgcccgagtcgtcgacgtggctccagactctgtggttattgaatccacaggcaccccaggcaagctccgcgcactgcttgacgtgatggaaccattcggaatccgcgaactgatccaatccggacagattgcactcaaccgcggtccgaagaccatggctccggccaagatctaa

SEQ ID NO: 40 - protein, 172 aa, Corynebacterium glutamicum ATCC 13032
MANSDVTRHILSVLVQDVDGIISRVSGMFTRRAFNLVSLVSAKTETHGINRITVVVDADELNIEQITKQLNKLIPVLKVVRLDEETTIARAIMLVKVSADSTNRPQIVDAANIFRARVVDVAPDSVVIESTGTPGKLRALLDVMEPFGIRELIQSGQIALNRGPKTMAPAKI

SEQ ID NO: 41 - DNA, 35 bp, Artificial Sequence
  synthetic oligonucleotide
ctgctcccaaggatcgcatcataacggttctggca

SEQ ID NO: 42 - DNA, 36 bp, Artificial Sequence
  synthetic oligonucleotide
ccgtcgacgatatcatatgatatctcctgtgtgaaa

SEQ ID NO: 43 - DNA, 39 bp, Artificial Sequence
  synthetic oligonucleotide
tgatatcgtcgacggatcctgcctggcggcagtagcgcg

SEQ ID NO: 44 - DNA, 35 bp, Artificial Sequence
  synthetic oligonucleotide
gaggcagacctcgataaaaaggccatccgtcagga

SEQ ID NO: 45 - DNA, 26 bp, Artificial Sequence
  synthetic oligonucleotide
ctagaaggacagtatttggtatctgc

SEQ ID NO: 46 - DNA, 26 bp, Artificial Sequence
  synthetic oligonucleotide
atactgtccttctagtgtagccgtag

SEQ ID NO: 47 - DNA, 34 bp, Artificial Sequence
  synthetic oligonucleotide
acaggagatatcatatgacgtcattagagttcac

SEQ ID NO: 48 - DNA, 41 bp, Artificial Sequence
  synthetic oligonucleotide
ccgccaggcaggatccagatcttagccaaccagtgggtaaa

SEQ ID NO: 49 - DNA, 27 bp, Artificial Sequence
  synthetic oligonucleotide
attcaggcagcagacgagctttacggt

SEQ ID NO: 50 - DNA, 26 bp, Artificial Sequence
  synthetic oligonucleotide
gtctgctgcctgaatgagccctccag

SEQ ID NO: 51 - DNA, 27 bp, Artificial Sequence
  synthetic oligonucleotide
attcagtgcgcagacgagctttacggt

SEQ ID NO: 52 - DNA, 26 bp, Artificial Sequence
  synthetic oligonucleotide
gtctgcgcactgaatgagccctccag

SEQ ID NO: 53 - DNA, 26 bp, Artificial Sequence
  synthetic oligonucleotide
tatacctccactgacatgggaactac

SEQ ID NO: 54 - DNA, 26 bp, Artificial Sequence
  synthetic oligonucleotide
gtcagtggaggtatagaagcgcccat

SEQ ID NO: 55 - DNA, 27 bp, Artificial Sequence
  synthetic oligonucleotide
attcagggcgcagacgagctttacggt

SEQ ID NO: 56 - DNA, 26 bp, Artificial Sequence
  synthetic oligonucleotide
gtctgcgccctgaatgagccctccag

SEQ ID NO: 57 - DNA, 34 bp, Artificial Sequence
  synthetic oligonucleotide
acaggagatatcatatgattttggtaactttaga

SEQ ID NO: 58 - DNA, 36 bp, Artificial Sequence
  synthetic oligonucleotide
ccgccaggcaggatccttactgatgaaaattccatt

SEQ ID NO: 59 - DNA, 34 bp, Artificial Sequence
  synthetic oligonucleotide
acaggagatatcatatgagactcgacaagacatt

SEQ ID NO: 60 - DNA, 35 bp, Artificial Sequence
  synthetic oligonucleotide
ccgccaggcaggatcctatacgcggagcggagtcg

SEQ ID NO: 61 - DNA, 27 bp, Artificial Sequence
  synthetic oligonucleotide
ccagctaagggaggtgcacgcatgtgg

SEQ ID NO: 62 - DNA, 27 bp, Artificial Sequence
  synthetic oligonucleotide
acctcccttagctggtcccagagtagt

SEQ ID NO: 63 - DNA, 33 bp, Artificial Sequence
  synthetic oligonucleotide
ggcctgatcaactccgccgacgagctttacggc

SEQ ID NO: 64 - DNA, 33 bp, Artificial Sequence
  synthetic oligonucleotide
ggcggagttgatcaggccaccagcgttgatgac

SEQ ID NO: 65 - DNA, 26 bp, Artificial Sequence
  synthetic oligonucleotide
atcaccggcgaagatgtgggcacaac

SEQ ID NO: 66 - DNA, 26 bp, Artificial Sequence
  synthetic oligonucleotide
atcttcgccggtgatgtatcggccgt

SEQ ID NO: 67 - DNA, 35 bp, Artificial Sequence
  synthetic oligonucleotide
gacggccagtgaattctcattccagcgtcacgacg

SEQ ID NO: 68 - DNA, 26 bp, Artificial Sequence
  synthetic oligonucleotide
ctcgaggggtagagccttttgttggt

SEQ ID NO: 69 - DNA, 35 bp, Artificial Sequence
  synthetic oligonucleotide
gctctacccctcgagaacaggaagagcccgtaaac

SEQ ID NO: 70 - DNA, 35 bp, Artificial Sequence
  synthetic oligonucleotide
taccgagctcgaattgggaagactcgcctctgcgg

SEQ ID NO: 71 - DNA, 33 bp, Artificial Sequence
  synthetic oligonucleotide
acaatgaccctttgaccgcctatgctaacgcac

SEQ ID NO: 72 - DNA, 33 bp, Artificial Sequence
  synthetic oligonucleotide
tcaaagggtcattgtcacggccgtcaacggtga

SEQ ID NO: 73 - DNA, 35 bp, Artificial Sequence
  synthetic oligonucleotide
ggctctacccctcgagaagaaatttagatgattga

SEQ ID NO: 74 - DNA, 33 bp, Artificial Sequence
  synthetic oligonucleotide
tcttcctgttctcgattcaccgacaaacaacag

SEQ ID NO: 75 - DNA, 25 bp, Artificial Sequence
  synthetic oligonucleotide
ggtaccgaagaaatttagatgattg

SEQ ID NO: 76 - DNA, 26 bp, Artificial Sequence
  synthetic oligonucleotide
actagtaaaaaggccatccgtcagga

SEQ ID NO: 77 - DNA, 35 bp, Artificial Sequence
  synthetic oligonucleotide
gtgaattcgagctcgtcacaccttctgccacgtca

SEQ ID NO: 78 - DNA, 43 bp, Artificial Sequence
  synthetic oligonucleotide
aatttcttcggtaccaccttgattgatcatgtcgaggaaagcc

SEQ ID NO: 79 - DNA, 35 bp, Artificial Sequence
  synthetic oligonucleotide
ggcctttttactagttgaattcgccgtcagttccg

SEQ ID NO: 80 - DNA, 35 bp, Artificial Sequence
  synthetic oligonucleotide
aggtggatacggatcaggctattgtgtctatcagg

SEQ ID NO: 81 - DNA, 35 bp, Artificial Sequence
  synthetic oligonucleotide
accaagtcagggatcgaagaaatttagatgattga

SEQ ID NO: 82 - DNA, 35 bp, Artificial Sequence
  synthetic oligonucleotide
ccgccaggcaggatccaactacattattgttctac

SEQ ID NO: 83 - DNA, 35 bp, Artificial Sequence; identical to SEQ ID NO: 82
  synthetic oligonucleotide

SEQ ID NO: 84 - DNA, 36 bp, Artificial Sequence
  synthetic oligonucleotide
agaggagacaccatatgaacgtggcagcttctcaac

SEQ ID NO: 85 - DNA, 34 bp, Artificial Sequence
  synthetic oligonucleotide
agaggagacaccataatgaatgtggcagcttctc

SEQ ID NO: 86 - DNA, 33 bp, Artificial Sequence
  synthetic oligonucleotide
ggaaaagtcatcgtctacgtcctgaacgagtac

SEQ ID NO: 87 - DNA, 33 bp, Artificial Sequence
  synthetic oligonucleotide
gacgatgacttttcccgcgtatcaggtatgttc

SEQ ID NO: 88 - DNA, 33 bp, Artificial Sequence
  synthetic oligonucleotide
gtcgacgatatcatattaagcggtttctgcgcg

SEQ ID NO: 89 - DNA, 32 bp, Artificial Sequence
  synthetic oligonucleotide
gtgaattcgagctcgatgcgcagcagacgagg

SEQ ID NO: 90 - DNA, 28 bp, Artificial Sequence
  synthetic oligonucleotide
gatttagctagccaatttcgtctcacgg

SEQ ID NO: 91 - DNA, 27 bp, Artificial Sequence
  synthetic oligonucleotide
ttggctagctaaatcaaccggttttaa

SEQ ID NO: 92 - DNA, 31 bp, Artificial Sequence
  synthetic oligonucleotide
aggtggatacggatcgcgaggtgtaatacgc

(19) United States
(12) Patent Application Publication
(10) Pub. No.: US 2024/0376483 A1
(43) Pub. Date: Nov. 14, 2024

(54) RECOMBINANT MICROORGANISM AND L-LEUCINE PRODUCTION METHOD USING SAME

(71) Applicants: Green Earth Institute Co., Ltd., Tokyo (JP); ITOCHU CHEMICAL FRONTIER Corporation, Tokyo (JP)

(72) Inventor: Shuhei NAKANE, Chiba (JP)

(73) Assignees: Green Earth Institute Co., Ltd., Tokyo (JP); ITOCHU CHEMICAL FRONTIER Corporation, Tokyo (JP)

(21) Appl. No.: 18/566,084

(22) PCT Filed: May 24, 2022
(86) PCT No.: PCT/JP2022/021200

§ 371 Date: Dec. 1, 2023

(30) Foreign Application Priority Data
Jun. 3, 2021 (JP) 2021-093831

(51) Int. Cl. C12N 15/74; C12N 1/20; C12N 9/06; C12N 9/10; C12P 13/06; C12R 1/15

(52) U.S. Cl. CPC C12N 15/74; C12N 1/20; C12N 9/0016; C12N 9/1022; C12N 9/1025; C12P 13/06; C12R 2001/15; C12Y 104/01005; C12Y 104/01009; C12Y 202/01006; C12Y 203/03013

(57) ABSTRACT

The present disclosure relates to a recombinant microorganism capable of efficiently producing L-leucine and an L-leucine production method using the same. The production method includes: (p) incubating a recombinant microorganism having an L-leucine biosynthesis pathway or a processed product of microbial cells thereof in a predetermined culture medium (X) to thereby produce L-leucine; and (q) recovering a fraction including the L-leucine from the predetermined culture medium (X). The recombinant microorganism includes, in an expressible form, a gene encoding an amino acid dehydrogenase. The amino acid dehydrogenase at least has a catalytic effect on an L-leucine production reaction in the L-leucine biosynthesis pathway.

### TECHNICAL FIELD

The present invention relates to a recombinant microorganism having an ability to produce L-leucine and an L-leucine production method using the same. More specifically, the present invention relates to a recombinant microorganism which has an ability to produce L-leucine and results in a reduced L-valine production amount relative to an L-leucine production amount, and an L-leucine production method using the same.

### BACKGROUND ART

In production of substances using microorganisms, attempts have been made to improve production efficiencies of various target substances, for example, by way of enhancing the activity of metabolic enzymes, or inactivating the activity of some metabolic enzymes, in metabolic systems involved in production of target substances from plant-derived sugars, based on gene recombination techniques or the like. For example, production techniques using microorganisms have been developed for many amino acids, and L-leucine production techniques are no exception.

For example, an L-leucine production method in which a mutant of Arthrobacter citreus that has resistance to azaleucine, which is a homologue of L-leucine, is cultured under aerobic conditions to produce a fermentation solution, and L-leucine accumulated is recovered from the fermentation solution has been known (Patent Document 1).

In addition, an L-leucine production method based on the finding that, in a case where microbial cells obtained by way of culturing conventionally known L-leucine producing bacteria in a culture medium containing sugars serving as a main carbon source are allowed to act on sugars and acetic acid or salts thereof, the accumulation of L-leucine is increased while the secondary production of L-valine is reduced, compared to a case where the microbial cells are allowed to act only on sugars, has been known (Patent Document 2).

Moreover, an L-leucine production method using Escherichia bacteria in which the ilvE gene encoding a branched-chain amino acid transaminase is inactivated and the activity of an aromatic amino acid transaminase encoded by the tyrB gene is enhanced has also been known (Patent Document 3).

Furthermore, Corynebacterium glutamicum mutant strains that have resistance to L-leucine and norleucine, which is a derivative of L-leucine, and that have an ability to produce L-leucine, and an L-leucine production method using the same have been known (Patent Document 4).

Furthermore, a fermentation production method for branched-chain amino acids using recombinant microorganisms that express acetohydroxyacid synthase (AHAS) mutants having resistance to feedback inhibition by amino acids has also been known (Patent Document 8).

In addition, Non-Patent Document 5 describes an L-leucine production method using Corynebacterium glutamicum mutant strains into which a mutant type 2-isopropylmalate synthase having resistance to feedback inhibition by L-leucine is introduced in addition to various gene modifications.

### CITATION LIST

### Patent Document

### Patent Document 1

Japanese Patent Laid-Open No. S58-220692

### Patent Document 2

Japanese Patent Laid-Open No. H08-266295

### Patent Document 3

Japanese Patent Laid-Open No. 2004-024259

### Patent Document 4

Published Japanese Translation No. 2018-525012 of the PCT International Publication

### Patent Document 5

Chinese Patent Application Publication No. CN110607290A

### Patent Document 6

Japanese Patent Laid-Open No. 2006-271379

### Patent Document 7

Japanese Patent Laid-Open No. 2008-61590

### Patent Document 8

Published Japanese Translation No. 2007-506407 of the PCT International Publication

### Non-Patent Document

### Non-Patent Document 1

FEBS Lett. (1995) 370:93-96

### Non-Patent Document 2

Biosci. Biotechnol. Biochem. (2009) 73:729-732

### Non-Patent Document 3

Eur. J. Biochem. (2003) 270:4628-4634

### Non-Patent Document 4

Encyclopedia of Industrial Biotechnology: Bioprocess, Bioseparation, and Cell Technology, edited by Michael C. Flickinger, “PHENYLALANINE DEHYDROGENASE”, YASUHISA ASANO

### Non-Patent Document 5

Schriften des Forschungszentrums Julich Reihe Gesundheit/Health, Band/Volume 73, “Metabolic engineering of Corynebacterium glutamicum for production of L-leucine and 2-ketoisocaproate.” Michael Vogt

### SUMMARY OF INVENTION

### Technical Problem

An objective of the present invention is to provide a recombinant microorganism capable of efficiently producing L-leucine and an L-leucine production method using the same.

Meanwhile, since Patent Documents 5 to 6 and Non-Patent Documents 1 to 4 disclose various mutant type amino acid dehydrogenases into which predetermined amino acid substitutions have been incorporated, these documents are listed above as conventional art documents. However, these conventional art documents do not describe or suggest use of the mutant type amino acid dehydrogenases for L-leucine production.

### Solution to Problem

According to aspects of the present invention, the followings are provided.

[1] A method for producing L-leucine, including:
(p) incubating a recombinant microorganism having an L-leucine biosynthesis pathway or a processed product of microbial cells thereof in a predetermined culture medium (X) to thereby produce L-leucine; and
(q) recovering a fraction including the L-leucine from the predetermined culture medium (X),
wherein the recombinant microorganism includes in an expressible form a gene encoding an amino acid dehydrogenase, and
wherein the amino acid dehydrogenase has at least a catalytic effect on an L-leucine production reaction in the L-leucine biosynthesis pathway.

[2] The method according to [1], wherein the L-leucine production reaction in the L-leucine biosynthesis pathway is a reaction represented by the following reaction formula (IX-1) or (IX-2):

$$[ Chem . 1 ]$$

$$4 - methyl - 2 - oxopentanoic acid + {NH}_{3} + NADH + {H}^{+} ⇔ L - leucine + {H}_{2} O + {NAD}^{+} \qquad ( IX - 1 )$$

$$[ Chem . 2 ]$$

$$4 - methyl - 2 - oxopentanoic acid + {NH}_{3} + NADH + {H}^{+} ⇔ L - leucine + {H}_{2} O + {NADP}^{+} . \qquad ( IX - 2 )$$

[3] The method according to [2], wherein the L-leucine production reaction in the L-leucine biosynthesis pathway is the reaction represented by the reaction formula (IX-1). [4] The method according to any one of [1] to [3], wherein the L-leucine biosynthesis pathway is a metabolic pathway capable of synthesizing L-leucine via 3-methyl-2-oxobutanoic acid and 4-methyl-2-oxopentanoic acid serving as intermediate metabolites, and the amino acid dehydrogenase has a catalytic effect on the reaction that produces L-leucine from 4-methyl-2-oxopentanoic acid.

[5] The method according to any one of [1] to [4], wherein the amino acid dehydrogenase is a wild type bacterial amino acid dehydrogenase or a mutant type amino acid dehydrogenase thereof. [6] The method according to any one of [1] to [5], wherein the amino acid dehydrogenase is a wild type amino acid dehydrogenase derived from Lysinibacillus sphaericus, Sporosarcina ureae or Rhodococcus jostii or a mutant type amino acid dehydrogenase thereof.

[7] The method according to any one of [1] to [6], wherein the amino acid dehydrogenase is a mutant type amino acid dehydrogenase having at least one of amino acid substitutions of the following (a), (b) and (c) based on the amino acid sequence shown in SEQ ID No. 2:
(a) an amino acid substitution of an amino acid corresponding to the 51st leucine (L) with lysine (K);
(b) an amino acid substitution of an amino acid corresponding to the 124th glycine (G) with serine(S); and
(c) an amino acid substitution of an amino acid corresponding to the 310th valine (V) with alanine (A), serine(S) or glycine (G);
with the proviso that, in each of (a) to (c) above, the amino acid before substitution and the amino acid after substitution are different from each other, and the mutant type amino acid dehydrogenase has catalytic activity for the reaction represented by the above reaction formula (IX-1) or (IX-2) or has enzyme activity defined in EC1.4.1.9.

[8] The method according to any one of [1] to [6], wherein the amino acid dehydrogenase is a mutant type amino acid dehydrogenase having at least one of amino acid substitutions of the following (d), (e), (f) and (g) based on the amino acid sequence shown in SEQ ID No. 26:
(d) an amino acid substitution of an amino acid corresponding to the 40th leucine (L) with lysine (K);
(e) an amino acid substitution of an amino acid corresponding to the 113th alanine (A) with glycine (G);
(f) an amino acid substitution of an amino acid corresponding to the 291st valine (V) with leucine (L); and
(g) an amino acid substitution of an amino acid corresponding to the 294th valine (V) with alanine (A), serine(S) or glycine (G),
with the proviso that, in each of (d) to (g) above, the amino acid before substitution and the amino acid after substitution are different from each other, and the mutant type amino acid dehydrogenase has catalytic activity for the reaction represented by the above reaction formula (IX-1) or (IX-2) or has enzyme activity defined in EC1.4.1.9.

[9] The method according to any one of [1] to [6], wherein the amino acid dehydrogenase consists of an amino acid sequence shown in any one of the following (A) to (C) and has catalytic activity for the reaction represented by the above reaction formula (IX-1) or (IX-2) or has enzyme activity defined in EC1.4.1.9:
(A) an amino acid sequence shown in SEQ ID No. 2, 10, 18 or 26;
(B) an amino acid sequence having a deletion, substitution and/or addition of one or more amino acids relative to the amino acid sequence set forth in (A) above; and
(C) an amino acid sequence having at least 60% sequence identity to the amino acid sequence set forth in (A) above.

[10] The method according to [7], wherein the amino acid dehydrogenase consists of an amino acid sequence shown in any one of the following (D) to (F) and has catalytic activity for the reaction represented by the above reaction formula (IX-1) or (IX-2) or has enzyme activity defined in EC1.4.1.9:
(D) an amino acid sequence having at least one of the amino acid substitutions of (a), (b) and (c) above relative to the amino acid sequence shown in SEQ ID No. 2;
(E) an amino acid sequence having a deletion, substitution and/or addition of one or more amino acids relative to the amino acid sequence set forth in (D) above; and
(F) an amino acid sequence having at least 60% sequence identity to the amino acid sequence set forth in (D) above,
with the proviso that, in the amino acid sequences set forth in (E) and (F) above, the amino acid substitutions set forth in (D) above are maintained.

[11] The method according to [8], wherein the amino acid dehydrogenase consists of an amino acid sequence shown in any one of the following (D) to (F) and has catalytic activity for the reaction represented by the above reaction formula (IX-1) or (IX-2) or has enzyme activity defined in EC1.4.1.9:
(D) an amino acid sequence having at least one of the amino acid substitutions of (d), (e), (f) and (g) above relative to SEQ ID No. 26;
(E) an amino acid sequence having a deletion, substitution and/or addition of one or more amino acids relative to the amino acid sequence set forth in (D) above; and
(F) an amino acid sequence having at least 60% sequence identity to the amino acid sequence set forth in (D) above,
with the proviso that, in the amino acid sequences set forth in (E) and (F) above, the amino acid substitutions set forth in (D) above are maintained.

[12] The method according to any one of [1] to [6], wherein the amino acid dehydrogenase consists of an amino acid sequence shown in any one of the following (G1) to (H) and has catalytic activity for the reaction represented by the above reaction formula (IX-1) or (IX-2) or has enzyme activity defined in EC1.4.1.9:
(G1) an amino acid sequence shown in SEQ ID No. 18;
(G2) an amino acid sequence having an amino acid substitution of V310A or V310C relative to the amino acid sequence shown in SEQ ID No. 2;
(G3) an amino acid sequence having a combination of amino acid substitutions of G124S and V310A or V310G relative to the amino acid sequence shown in SEQ ID No. 2;
(G4) an amino acid sequence having a combination of amino acid substitutions of L40K. A113G, V291L, and V294S relative to the amino acid sequence shown in SEQ ID No. 26; and
(H) an amino acid sequence having a deletion, substitution and/or addition of one or more amino acids relative to the amino acid sequence set forth in any one of (G1) to (G4) above,
with the proviso that, in (H) above, the amino acid substitutions set forth in (G2) to (G4) above are maintained.

[13] The method according to any one of [1] to [12], wherein a gene encoding the amino acid dehydrogenase has been introduced into the recombinant microorganism in a forcibly expressible manner so that the recombinant microorganism exhibits a production ratio of L-leucine/L-valine increased compared to that of a control microorganism.

[14] The method according to any one of [1] to [13], wherein the recombinant microorganism has reduced or inactivated activity of at least one of enzymes defined in EC1.4.1.8, EC1.4.1.23. EC2.6.1.42 and EC2.6.1.66 among enzymes that catalyze a reaction secondarily producing L-valine from 3-methyl-2-oxobutanoic acid.

[15] The method according to any one of [1] to [14], wherein the recombinant microorganism has reduced or inactivated activity of an enzyme defined in EC2.6.1.42 among enzymes that catalyze a reaction secondarily producing L-valine from 3-methyl-2-oxobutanoic acid.

[16] The method according to any one of [1] to [15], wherein the recombinant microorganism is a recombinant coryneform bacterium that satisfies at least one of the following conditions (i), (iv) and (x), and the following condition (ix) (preferably a recombinant coryneform bacterium that satisfies all of the following conditions (i), (iv) and (x), and condition (ix)):
(i) a gene encoding an enzyme specified in EC2.2.1.6 has been introduced thereinto in a forcibly expressible manner;
(iv) a gene encoding an enzyme specified in EC2.3.3.13 has been introduced thereinto in forcibly expressible manner;
(x) a gene encoding an enzyme that catalyzes a reaction secondarily producing L-valine from 3-methyl-2-oxobutanoic acid has been inactivated or deficient therein; and
(ix) a gene encoding the amino acid dehydrogenase has been introduced into the recombinant microorganism in a forcibly expressible manner so that the recombinant microorganism exhibits a production ratio of L-leucine/L-valine increased compared to that of a control microorganism.

[17] The method according to any one of [1] to [16], wherein the recombinant microorganism is a recombinant coryneform bacterium that satisfies at least one of the following conditions (i), (iv) and (x), and the following condition (ix):
(i) an ilvBN gene, an ortholog thereof, or a mutant type gene of any of these has been introduced thereinto in a forcibly expressible manner;
(iv) a leuA gene, an ortholog thereof, or a mutant type gene of any of these has been introduced in a forcibly expressible manner;
(x) an ilvE gene or an ortholog thereof present on the genome has been inactivated or deficient therein; and
(ix) a gene encoding the amino acid dehydrogenase has been introduced into the recombinant microorganism in a forcibly expressible manner so that the recombinant microorganism exhibits a production ratio of L-leucine/L-valine increased compared to that of a control microorganism.

[18] The method according to any one of [1] to [17], wherein the recombinant microorganism is a recombinant coryneform bacterium that satisfies at least one of the following conditions (i), (iv) and (x), and the following condition (ix):
(i) a mutant type ilvBN gene encoding an acetolactate synthase that has resistance to feedback inhibition of enzyme activity by at least one of valine, leucine and isoleucine has been introduced thereinto in a forcibly expressible manner;
(iv) a mutant type leuA gene encoding a mutant type 2-isopropylmalate synthase that has resistance to feedback inhibition of enzyme activity by L-leucine has been introduced thereinto in a forcibly expressible manner;
(x) an ilvE gene or an ortholog thereof present on the genome has been inactivated or deficient therein; and
(ix) a gene for encoding the amino acid dehydrogenase has been introduced into the recombinant microorganism in a forcibly expressible manner so that the recombinant microorganism exhibits a production ratio of L-leucine/L-valine increased compared to that of a control microorganism.

[19] The method according to [18], wherein the recombinant microorganism satisfies all of the above conditions (i), (iv) and (x), a gene encoding a protein defined in the following (J1) or (J2) and a gene encoding a protein defined in the following (K3) or (K4) serving as the mutant type ilvBN gene in (i) above have been introduced thereinto in a forcibly expressible manner, and a gene encoding a protein defined in either the following (L3) or (L4) serving as the mutant type leuA gene in (iv) above has introduced thereinto in a forcibly expressible manner:
(J1) a protein consisting of an amino acid sequence shown in SEQ ID No. 38;
(J2) a protein consisting of an amino acid sequence having a deletion, substitution and/or addition of one or more amino acids relative to the amino acid sequence shown in SEQ ID No. 38. the protein being capable of functioning as an acetolactate synthase large subunit;
(K3) a protein consisting of an amino acid sequence having amino acid substitutions of G20D, I21D and I22F relative to the amino acid sequence shown in SEQ ID No. 38, the protein functioning as an acetolactate synthase small subunit and having resistance to feedback inhibition by at least one of valine, leucine and isoleucine;
(K4) a protein consisting of an amino acid sequence having a deletion, substitution and/or addition of one or more amino acids relative to the amino acid sequence of the protein set forth in (K3) above (with the proviso that the amino acid substitutions of G20D, I21D and I22F are maintained, and the protein functions as an acetolactate synthase small subunit and has resistance to feedback inhibition of enzyme activity by at least one of valine, leucine and isoleucine);
(L3) a protein consisting of an amino acid sequence having amino acid substitutions of G530D. G532D and A535T relative to the amino acid sequence shown in SEQ ID No. 36, the protein having enzyme activity specified in EC2.3.3.13 and resistance to feedback inhibition of enzyme activity by L-leucine; and
(L4) a protein consisting of an amino acid sequence having a deletion, substitution and/or addition of in which one or more amino acids relative to the amino acid sequence of the protein defined in (L3) above (with the proviso that amino acid substitutions of G530D, G532D and A535T are maintained, and the protein has enzyme activity specified in EC2.3.3.13 and resistance to feedback inhibition of enzyme activity by L-leucine).

[20] The method according to any one of [1] to [19], wherein the amino acid dehydrogenase has catalytic activity for the reaction represented by the reaction formula (IX-1) or has enzyme activity defined in EC1.4.1.9.

[21] The method according to any one of [1] to [20], wherein, in step (p), the recombinant microorganism is cultured under aerobic conditions in the predetermined culture medium (X) to thereby produce L-leucine.

[22] The method according to any one of [1] to [21], wherein a gene encoding the amino acid dehydrogenase has been introduced into the recombinant microorganism in a forcibly expressible manner so that the recombinant microorganism exhibits a production ratio of L-leucine/L-valine and/or L-leucine/L-isoleucine increased compared to that/those of a control microorganism.

[23] A recombinant microorganism (preferably coryneform bacteria), including an L-leucine biosynthesis pathway via 3-methyl-2-oxobutanoic acid and 4-methyl-2-oxopentanoic acid serving as intermediate metabolites, and satisfying all of the following conditions (i), (iv), (x) and (ix):
(i) a mutant type ilvBN gene encoding an acetolactate synthase that has resistance to feedback inhibition of enzyme activity by at least one of valine, leucine and isoleucine has been introduced thereinto in a forcibly expressible manner;
(iv) a mutant type leuA gene encoding a mutant type 2-isopropylmalate synthase that has resistance to feedback inhibition of enzyme activity by L-leucine has been introduced thereinto in a forcibly expressible manner;
(x) an ilvE gene or an ortholog thereof present on the genome has been inactivated or deficient therein; and
(ix) a gene encoding a bacterial amino acid dehydrogenase has been introduced into the recombinant microorganism in a forcibly expressible manner so that the recombinant microorganism exhibits a production ratio of L-leucine/L-valine increased compared to that of a control microorganism.

[24] The recombinant microorganism (preferably coryneform bacteria) according to [23], wherein the bacterial amino acid dehydrogenase in the condition (ix) consists of an amino acid sequence shown in the following (A) or (B), and has catalytic activity for the reaction represented by the following reaction formula (IX-1) or (IX-2) or has enzyme activity defined in EC1.4.1.9:
(A) an amino acid sequence shown in SEQ ID No. 18; and
(B) an amino acid sequence having a deletion, substitution and/or addition of more amino acids relative to the amino acid sequence set forth in (A) above:

$$[ Chem . 3 ]$$

$$4 - methyl - 2 - oxopentanoic acid + {NH}_{3} + NADH + {H}^{+} ⇔ L - leucine + {H}_{2} O + {NAD}^{+} \qquad ( IX - 1 )$$

$$[ Chem . 4 ]$$

$$4 - methyl - 2 - oxopentanoic acid + {NH}_{3} + NADH + {H}^{+} ⇔ L - leucine + {H}_{2} O + {NADP}^{+} . \qquad ( IX - 2 )$$

[25] The recombinant microorganism (preferably coryneform bacteria) according to [23], wherein the bacterial amino acid dehydrogenase in the condition (ix) consists of an amino acid sequence shown in any one of the following (D) to (F), and has catalytic activity for the reaction represented by the following reaction formula (IX-1) or (IX-2) or has enzyme activity defined in EC1.4.1.9:
(D) an amino acid sequence having at least one of the amino acid substitutions defined in the following (a), (b) and (c) relative to the amino acid sequence shown in SEQ ID No. 2,
(a) an amino acid substitution of an amino acid corresponding to the 51st leucine (L) with lysine (K),
(b) an amino acid substitution of an amino acid corresponding to the 124th glycine (G) with serine(S), and
(c) an amino acid substitution of an amino acid corresponding to the 310th valine (V) with alanine (A), serine(S) or glycine (G), with the proviso that, in each of (a) to (c) above, the amino acid before substitution and the amino acid after substitution are different from each other;
(E) an amino acid sequence having a deletion, substitution and/or addition of one or more amino acids relative to the amino acid sequence set forth in (D) above; and
(F) an amino acid sequence having at least 60% sequence identity to the amino acid sequence set forth in (D) above,
with the proviso that, in the amino acid sequences defined in (E) and (F) above, the amino acid substitutions defined in (D) above are maintained:

$$[ Chem . 5 ]$$

$$4 - methyl - 2 - oxopentanoic acid + {NH}_{3} + NADH + {H}^{+} ⇔ L - leucine + {H}_{2} O + {NAD}^{+} \qquad ( IX - 1 )$$

$$[ Chem . 6 ]$$

$$4 - methyl - 2 - oxopentanoic acid + {NH}_{3} + NADH + {H}^{+} ⇔ L - leucine + {H}_{2} O + {NADP}^{+} . \qquad ( IX - 2 )$$

[26] The recombinant microorganism (preferably coryneform bacteria) according to [23], wherein the bacterial amino acid dehydrogenase in the condition (ix) consists of an amino acid sequence shown in any one of the following (D) to (F), and has catalytic activity for the reaction represented by the following reaction formula (IX-1) or (IX-2) or has enzyme activity defined in EC1.4.1.9:
(D) an amino acid sequence having at least one of the amino acid substitutions of (d), (e), (f) and (g) above relative to SEQ ID No. 26;
(E) an amino acid sequence having a deletion, substitution and/or addition of one or more amino acids relative to the amino acid sequence set forth in (D) above; and
(F) an amino acid sequence having at least 60% sequence identity to the amino acid sequence set forth in (D) above,
with the proviso that, in the amino acid sequences set forth in (E) and (F) above, the amino acid substitutions set forth in (D) above are maintained:

$$[ Chem . 7 ]$$

$$4 - methyl - 2 - oxopentanoic acid + {NH}_{3} + NADH + {H}^{+} ⇔ L - leucine + {H}_{2} O + {NAD}^{+} \qquad ( IX - 1 )$$

$$[ Chem . 8 ]$$

$$4 - methyl - 2 - oxopentanoic acid + {NH}_{3} + NADH + {H}^{+} ⇔ L - leucine + {H}_{2} O + {NADP}^{+} . \qquad ( IX - 2 )$$

[27] The recombinant recombinant microorganism (preferably coryneform bacteria) according to [23], wherein the bacterial amino acid dehydrogenase in the condition (ix) consists of an amino acid sequence shown in any one of the following (G1) to (H), and has catalytic activity for the reaction represented by the following reaction formula (IX-1) or (IX-2) or has enzyme activity defined in EC1.4.1.9:
(G1) an amino acid sequence shown in SEQ ID No. 18;
(G2) an amino acid sequence having an amino acid substitution of V310A or V310C relative to the amino acid sequence shown in SEQ ID No. 2;
(G3) an amino acid sequence having a combination of amino acid substitutions of G124S, and V310A or V310G relative to the amino acid sequence shown in SEQ ID No. 2;
(G4) an amino acid sequence having a combination of amino acid substitutions of L40K. A113G, V291L, and V294S relative to the amino acid sequence shown in SEQ ID No. 26; and
(H) an amino acid sequence having a deletion, substitution and/or addition of one or more amino acids relative to the amino acid sequence defined in any one of (G1) to (G4) above,
with the proviso that, in (H) above, the amino acid substitutions defined in (G2) to (G4) above are maintained:

$$[ Chem . 9 ]$$

$$4 - methyl - 2 - oxopentanoic acid + {NH}_{3} + NADH + {H}^{+} ⇔ L - leucine + {H}_{2} O + {NAD}^{+} \qquad ( IX - 1 )$$

$$[ Chem . 10 ]$$

$$4 - methyl - 2 - oxopentanoic acid + {NH}_{3} + NADH + {H}^{+} ⇔ L - leucine + {H}_{2} O + {NADP}^{+} . \qquad ( IX - 2 )$$

[28] The recombinant recombinant microorganism (preferably coryneform bacteria) according to any one of to [27], wherein a gene encoding a protein defined in the following (J1) or (J2) and a gene encoding a protein defined in the following (K3) or (K4) serving as the mutant type ilvBN gene in the condition (i) have been introduced into the recombinant microorganism in a forcibly expressible manner, and a gene encoding a protein defined in either the following (L3) or (L4) serving as the mutant leuA gene in the condition (iv) has been introduced into the recombinant microorganism in a forcibly expressible manner:
(J1) a protein consisting of an amino acid sequence shown in SEQ ID No. 38;
(J2) a protein consisting of an amino acid sequence having a deletion, substitution and/or addition of one or more amino acids relative the amino acid sequence shown in SEQ ID No. 38, the protein being capable of functioning as an acetolactate synthase large subunit:
(K3) a protein consisting of an amino acid sequence having amino acid substitutions of G20D, I21D and I22F relative to the amino acid sequence shown in SEQ ID No. 40, the protein functioning as an acetolactate synthase small subunit and having resistance to feedback inhibition by at least one of valine, leucine and isoleucine;
(K4) a protein consisting of an amino acid sequence having a deletion, substitution and/or addition of one or more amino acids relative to the amino acid sequence of the protein set forth in (K3) above (with the proviso that the amino acid substitutions of G20D, 121D and I22F are maintained, and the protein functions as an acetolactate synthase small subunit and has resistance to feedback inhibition of enzyme activity by at least one of valine, leucine and isoleucine);
(L3) a protein consisting of an amino acid sequence having amino acid substitutions of G530D, G532D and A535T relative to the amino acid sequence shown in SEQ ID No. 36, the protein having enzyme activity specified in EC2.3.3.13 and resistance to feedback inhibition of enzyme activity by L-leucine; and
(L4) a protein consisting of an amino acid sequence having a deletion, substitution and/or addition of one or more amino acids relative to the amino acid sequence of the protein set forth in (L3) above (with the proviso that the amino acid substitutions of G530D, G532D and A535T are maintained, and the protein has enzyme activity specified in EC2.3.3.13 and resistance to feedback inhibition of enzyme activity by L-leucine).

The recombinant recombinant microorganism (preferably coryneform bacteria) according to any one of [23] to [28], wherein the bacterial amino acid dehydrogenase in the condition (ix) has catalytic activity for the reaction represented by the reaction formula (IX-1) or has enzyme activity defined in EC1.4.1.9.

### Advantageous Effects of Invention

According to the present invention, efficient production of L-leucine can be realized in a bioprocess using a microorganism. In addition, in a specific embodiment of the present invention, it is possible to suppress secondary production of L-valine and L-isoleucine relative to the production of L-leucine, and thus, L-leucine can be more efficiently produced.

### BRIEF DESCRIPTION OF DRAWINGS

FIG. 1 is a schematic diagram showing an example of a leucine biosynthesis pathway that can be used in the present invention.

FIG. 2 is a diagram showing results obtained by performing multiple alignment (ClustalW) analysis on amino acid sequences of various amino acid dehydrogenases that can be used in the present invention.

FIG. 3 is a diagram showing results obtained by performing multiple alignment (ClustalW) analysis on amino acid sequences of various amino acid dehydrogenases that can be used in the present invention.

FIG. 4 is a diagram showing results obtained by performing multiple alignment

(ClustalW) analysis on amino acid sequences of various amino acid dehydrogenases that can be used in the present invention.

FIG. 5 is a diagram showing results obtained by performing multiple alignment (ClustalW) analysis on amino acid sequences of various amino acid dehydrogenases that can be used in the present invention.

FIG. 6 is a diagram showing results obtained by performing multiple alignment (ClustalW) analysis on amino acid sequences of various amino acid dehydrogenases that can be used in the present invention.

FIG. 7A is a diagram showing results (various amino acid concentrations) of a leucine production test in examples.

FIG. 7B is a diagram showing results (isoleucine concentration) of a leucine production test in examples.

FIG. 8A is a diagram showing results (leucine/valine production ratio) of a leucine production test in examples.

FIG. 8B is a diagram showing results (leucine/isoleucine production ratio) of a leucine production test in examples.

### DESCRIPTION OF EMBODIMENTS

Hereinafter, the meanings of terms in the present invention will be described and specific embodiments that can be used in the present invention will be exemplified.

Here, the meanings of abbreviations used in this specification are as follows.
DH: dehydrogenase
aaDH: amino acid dehydrogenase
pheDH: phenylalanine dehydrogenase
leuDH: leucine dehydrogenase
pheDH: phenylalanine dehydrogenase
pheDH: phenylalanine dehydrogenase derived from L. sphaericus
Rj_leuDH: R. jostii leucine dehydrogenase
Su_pheDH: S. ureae phenylalanine dehydrogenase
Ls_pheDH: L. sphaericus leucine dehydrogenase
Glu/Leu/Phe/Val DH: Glu/Leu/Phe/Val dehydrogenase
BCAA DH: branched-chain amino acid dehydrogenase.

### <Recombinant Microorganisms>

In the present invention, the term “recombinant microorganisms” may be understood literally, and may be understood as host microorganisms subjected to some kind of genetic manipulation. In addition, in the present invention, “microorganisms” or “recombinant microorganisms” may be specifically fungi or prokaryotes such as archaea, cyanobacteria, and bacteria. In the present invention, “microorganisms” or “recombinant microorganisms” are preferably fungi or bacteria, and more preferably bacteria.

Examples of fungi include yeasts of the genus Saccharomyces (for example, Saccharomyces cerevisiae), yeasts of the genus Schizosaccharomyces (for example, Schizosaccharomyces pombe), yeasts of the genus Pichia (for example, Pichia pastoris), yeasts of the genus Kluyveromyces (Kluyveromyces lactis), Hansenula polymorpha, yeasts of the genus Yarrowia (for example, Yarrowia lipolytica), fungi of the genus Cryptococcus (for example, Cryptococcus sp. S-2), fungi of the genus Aspergillus (for example, Aspergillus oryzae), and fungi of the genus of Pseudozyma (for example, Pseudozyma antarctica). Saccharomyces cerevisiae Schizosaccharomyces pombe, Pichia pastoris and the like can be conveniently used in the present invention because genetic manipulation techniques and heterologous protein expression systems have been established for these.

Examples of bacteria include bacteria of the genus Escherichia (for example, Escherichia coli), the genus Bacillus (for example, Bacillus subtilis), the genus Lactobacillus (for example, Lactobacillus acidophilus), the genus Clostridium (for example, Clostridium thermocellum, Clostridium acetobutylicum) the genus Rhodopseudomonas (for example, Rhodopseudomonas palustris), the genus Rhodobacter (for example, Rhodobacter capsulatus), the genus Pantoea (for example, Pantoea ananatis) and bacteria belonging to the coryneform bacteria detailed below. “Microorganisms” or “recombinant microorganisms” in the present invention are preferably Escherichia or coryneform bacteria, more preferably Escherichia coli or coryneform bacteria, and most preferably Corynebacterium bacteria, for which genetic manipulation techniques and protein expression systems have already been established, and which allow production of substances under anaerobic conditions or microaerobic conditions in which the cells do not substantially proliferate.

Here, “Coryneform bacteria” refers to a group of microorganisms defined in Bargeys Manual of Determinative Bacteriology (Vol. 8, p. 599, 1974).

More specifically, examples of coryneform bacteria include Corynebacterium bacteria (including the former Brevibacterium bacteria), Arthrobacter bacteria, Mycobacterium bacteria, Micrococcus bacteria, and Microbacterium bacteria.

Examples of Corynebacterium bacteria include the following species and strains. Corynebacterium glutamicum (for example, FERM P-18976 strains, ATCC13032 strains, ATCC31831 strains, ATCC13058 strains, ATCC13059 strains, ATCC13060 strains, ATCC13232 strains, ATCC13286 strains, ATCC13287 strains, ATCC13655 strains, ATCC13745 strains, ATCC13746 strains, ATCC13761 strains, and ATCC14020 strains);
Corynebacterium acetoglutamicum (for example, ATCC15806 strains);
Corynebacterium acetoacidophilum (for example, ATCC13870 strains);
Corynebacterium melassecola (for example, ATCC17965 strains);
Corynebacterium efficiens (for example, YS-314 strains, YS-314T strains (NBRC100395T strains));
Corynebacterium alkanolyticum (for example, ATCC21511 strains);
Corynebacterium callunae (for example, ATCC15991 strains, NBRC15359 strains, and DSM20147 strains);

Corynebacterium lilium (for example, ATCC15990 strains);
Corynebacterium thermoaminogenes (Corynebacterium efficiens) (for example, AJ12340 strains, and FERM BP1539 strains);
Corynebacterium herculis (for example, ATCC13868 strains);
Corynebacterium ammoniagenes (Brevibacterium ammoniagenes) (for example, ATCC6871 strains, ATCC6872 strains, DSM20306 strains, NBRC12071T strains, NBRC12072 strains, and NBRC12612T strains);
Corynebacterium pollutisoli;
Corynebacterium marinum (for example, DSM44953 strains);
Corynebacterium humireducens (for example, NBRC106098 strains);
Corynebacterium halotolerans (for example, YIM70093 strains);
Corynebacterium deserti (for example, GIMN1.010 strains);
Corynebacterium doosanense (for example, CAU212 strains, and DSM45436 strains); and
Corynebacterium maris (for example, DSM45190 strains).

Specific examples of Arthrobacter bacteria include the following species and strains. Arthrobacter globiformis (for example, ATCC8010 strains, ATCC4336 strains, ATCC21056 strains, ATCC31250 strains, ATCC31738 strains, ATCC35698 strains, NBRC3062 strains, and NBRC12137T strains) and the like may be exemplified.

Specific examples of Micrococcus bacteria include Micrococcus freudenreichii [for example, No. 239 (FERM P-13221) strains]; Micrococcus luteus [for example, NCTC2665 strains, No. 240 (FERM P-13222) strains]; Micrococcus ureae (for example, IAM1010 strains); Micrococcus roseus (for example, IFO3764 strains) and the like.

Specific examples of Microbacterium bacteria include Microbacterium ammoniaphilum (for example, ATCC15354 strains).

In the present invention, the meaning of “L-leucine biosynthesis pathway” in the “recombinant microorganism having an L-leucine biosynthesis pathway” may be interpreted literally, and it means that the recombinant microorganism according to the present invention has a metabolic pathway capable of biosynthesizing L-leucine.

In addition, the microorganism according to the present invention includes a gene encoding an amino acid dehydrogenase in an expressible form, and the amino acid dehydrogenase has a catalytic effect on an L-leucine production reaction in the L-leucine biosynthesis pathway. Therefore, the L-leucine biosynthesis pathway in the present invention includes, as a part thereof, an L-leucine production reaction that can be catalyzed by the amino acid dehydrogenase.

Here, the “amino acid dehydrogenase” may be interpreted literally, and refers to a concept that encompasses various amino acid dehydrogenases such as leucine dehydrogenase, phenylalanine dehydrogenase, branched-chain amino acid dehydrogenase, and Glu/Leu/Phe/Val dehydrogenase.

In a specific embodiment, the L-leucine production reaction that can be catalyzed by an amino acid dehydrogenase may be, but not particularly limited to, a reaction a reaction “production of L-leucine from 4-methyl-2-oxopentanoic acid” according to (ix) shown in Table 1. In addition, more specifically, the L-leucine production reaction that can be catalyzed by an amino acid dehydrogenase may be a reaction represented by the reaction formula (IX-1) or (IX-2) in (ix) of Table 1.

TABLE 1

| Reac- | | | | |
|---|---|---|---|---|
| tion | Reaction | | | Enzyme name/ |
| path | (inclusive | Reaction formula | | gene name |
| No. | concept) | (specific embodiment) | EC No. | (example) |
| (ix) | Production | 4-methyl-2-oxopentanoic | 1.4.1.9 | aaDH |
| | of L-leucine | acid + NH₃ + NADH + | (NAD⁺ | pheDH |
| | from 4- | H⁺ [symbol] L-leucine + | required | leuDH |
| | methyl-2- | H₂O + NAD⁺ (IX- | type) | Glu/Leu/ |
| | oxopentanoic | 1) | | Phe/Val DH |
| | acid | | | BCAA DH |
| | | | | yqiT |
| | | | | (gene name) |
| | | | | bcd (gene |
| | | | | name) |
| | | 4-methyl-2-oxopentanoic | - | Same as above |
| | | acid + NH₃ + NADPH + | (NADP⁺ | (mainly |
| | | H⁺ [symbol] L-leucine + | required | NADP+ |
| | | H₂O + NADP⁺ | type) | requiring |
| | | (IX-2) | | mutant type |
| | | | | amino acid |
| | | | | dehydrogenase) |

The reaction formula (IX-1) or (IX-2) in (ix) of Table 1 means a bidirectional reversible reaction between a deamination reaction of leucine and an amination reaction of 4-methyl-2-oxopentanoic acid, and the production of L-leucine from 4-methyl-2-oxopentanoic acid corresponds to the direction of the amination reaction of 4-methyl-2-oxopentanoic acid within recombinant microorganism cells. Moreover, the amino acid dehydrogenase that catalyzes the reaction according to the reaction formula (IX-1) has an enzyme activity specified by the enzyme number EC1.4.1.9 defined by the Enzyme Committee of the International Union of Biochemistry and Molecular Biology, and requires NAD serving as a coenzyme. Furthermore, the amino acid dehydrogenase which catalyzes the reaction according to the reaction formula (IX-2) requires NADP serving as a coenzyme. In addition, an enzyme that catalyzes either or both of the reactions according to the reaction formulae (IX-1) and (IX-2) may be used in the present invention.

Furthermore, the terms such as “catalyzing the reaction represented by the reaction formula (IX-1) or (IX-2),” “having catalytic activity for the reaction according to the reaction formula (IX-1) or (IX-2),” and “having catalytic activity for the reaction according to the reaction formula (IX-1) or (IX-2)” are synonymous, and may be used interchangeably herein. In addition, such terms do not exclude enzymes that catalyze both of the reactions according to the reaction formulae (IX-1) and (IX-2).

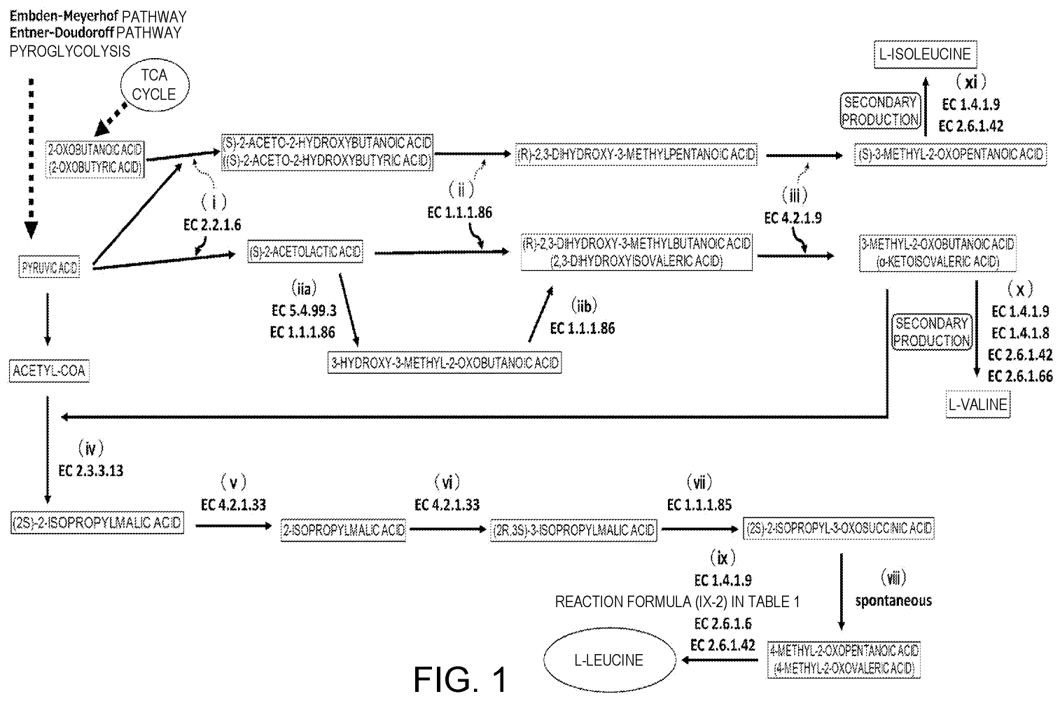

Additionally, FIG. 1 refers to an example of an L-leucine biosynthesis pathway that the recombinant microorganism according to the present invention may have, and the furthest downstream reaction path (ix) corresponds to (ix) in Table 1.

Additionally, in the present invention, needless to say, the amino acid dehydrogenase that catalyzes the reaction represented by the reaction formula (IX-1) or (IX-2) does not necessarily have to formally be assigned with an EC number such as EC1.4.1.9, and thus, it is only necessary to employ any amino acid dehydrogenases having catalytic activity for the reaction according to the reaction formula (IX-1) or (IX-2) even if they are not assigned with any EC numbers. In addition, the amino acid dehydrogenase does not exclude those having catalytic activity for other reactions in addition to the catalytic activity for the reaction according to the reaction formula (IX-1) or (IX-2).

In the present invention, regardless of the origin of amino acid dehydrogenase, for example, any wild type dehydrogenases isolated from organisms such as plants, fungi (for example, yeasts), archaea, and bacteria, any mutant type dehydrogenases thereof, or any chimeric dehydrogenases of these that have catalytic activity for the reaction according to the reaction formula (IX-1) or (IX-2) can be employed, and those having catalytic activity for the reaction according to the reaction formula (IX-1) (i.e., amino acid dehydrogenases having enzyme activity specified in EC1.4.1.9) may preferably be employed. In addition, among these amino acid dehydrogenases, it is more preferable to use a bacterial amino acid dehydrogenase, and it is particularly preferable to use a bacterial amino acid dehydrogenase having enzyme activity specified in EC1.4.1.9.

Additionally, while specific examples of bacterial amino acid dehydrogenases having enzyme activity specified in EC1.4.1.9 (reaction formula (IX-1)) are as described below, amino acid dehydrogenases that can catalyze the reaction according to the reaction formula (IX-2) may be little known among naturally occurring wild type amino acid dehydrogenases. However, such amino acid dehydrogenases can be artificially produced by those skilled in the art using genetic engineering or enzyme engineering techniques (for example, see Protein Eng. 1997 June; 10 (6): 687-90.).

In some embodiments, for the amino acid dehydrogenase, wild type amino acid dehydrogenases derived from bacteria belonging to the genus Lysinibacillus, the genus Bacillus, the genus Ornithinibacillus, the genus Oceanobacillus, the genus Virgibacillus, the genus Pseudogracilibacillus, the genus Salinicoccus, the genus Cytobacillus, the genus Mesobacillus, the genus Quasibacillus, the genus Peribacillus, the genus Geobacillus, the genus Rummeliibacillus, the genus Anoxybacillus, the genus Fictibacillus, the genus Psychrobacillus, the genus Tenuibacillus, the genus Jeotgalibacillus, the genus Lottiidibacillus, the genus Alteribacillus, the genus Aquisalibacillus, the genus Alkalibacillus, the genus Neobacillus, the genus Alkalihalobacillus, Filobacillus, the genus Lentibacillus, the genus Ureibacillus, the genus Pontibacillus, the genus Thalassobacillus, the genus Metasolibacillus, the genus Solibacillus, the genus Priestia, the genus Caldalkalibacillus, the genus Scopulibacillus, the genus Filibacter, the genus Parageobacillus, the genus Pradoshia, the genus Exiguobacterium, the genus Aliicoccus, the genus Sporosarcina, the genus Paenisporosarcina, the genus Weizmannia, the genus Rhodococcus, the genus Nocardia, the genus Mycolicibacterium, the genus Mycolicibacterium, the genus Pseudonocardia, the genus Clostridiumu, and the genus Thermoactinomyces, or mutant amino acid dehydrogenases thereof can be used.

In addition, more specifically, for the amino acid dehydrogenase, various bacterial amino acid dehydrogenases shown in Table 2 or mutants thereof can be used. However, such wild type amino acid dehydrogenases or mutants thereof shall have catalytic activity for the reaction according to the reaction formula (IX-1) or (IX-2) or shall have enzyme activity defined in EC1.4.1.9. In addition, in a specific embodiment, the amino acid dehydrogenase may be an amino acid dehydrogenase including an amino acid sequence shown in any one of SEQ ID Nos. 2 to 8, 10, 12 to 16, 18 to 22, 24, 26 to 30 and 32, or a mutant amino acid dehydrogenase thereof.

TABLE 2

| | | | | Sequence | |
|---|---|---|---|---|---|
| | | | | identity* | |
| | | | | (amino | |
| No. | Species name | Enzyme name | Accession No. | acid) | SEQ ID No |
| 1 | Lysinibacillus | pheDH | P23307.1 | — | 1 (nucleotide |
| | sphaericus | | | | sequence) |
| | | | | | 2 (amino acid |
| | | | | | sequence) |
| 2 | Bacillus | aaDH | WP_144463545.1 | 92.13% | 3 (amino acid |
| | MULTISPECIES | | | | sequence) |
| 3 | Virgibacillus | aaDH | WP_029268816.1 | 78.84% | 4 (same as |
| | alimentarius | | | | above) |
| 4 | Oceanobacillus limi | pheDH | SET60028.1 | 75.07% | 5 (same as |
| | | | | | above) |
| 5 | Salinicoccus roseus | Glu/Leu/Phe/Val | WP_040106347.1 | 71.31% | 6 (same as |
| | | DH | | | above) |
| 6 | Priestia abyssalis | leuDH | WP_078411368.1 | 70.11% | 7 (same as |
| | | | | | above) |
| 7 | Weizmannia coagulans | Glu/Leu/Phe/Val | WP_035188200.1 | 69.38% | 8 (same as |
| | | DH | | | above) |
| 8 | Sporosarcina ureae | aaDH | WP_029052528.1 | — | 9 (nucleotide |
| | | | | | sequence) |
| | | | | | 10 (amino acid |
| | | | | | sequence) |
| 9 | Sporosarcina ureae | pheDH | P97014.1 | 96.83% | 11 (nucleotide |
| | R-04 | | | | sequence) |
| | | | | | 12 (amino acid |
| | | | | | sequence) |
| 10 | Sporosarcina sp. P3 | aaDH | WP_099638561.1 | 96.31% | 13 (amino acid |
| | | | | | sequence) |
| 11 | Bacillus sp. OxB-1 | Glu/Leu/Phe/Val | WP_052484069.1 | 86.51% | 14 (same as |
| | | DH | | | above) |
| 12 | Bacillus sp. FJAT- | Glu/Leu/Phe/Val | WP_028397941.1 | 70.92% | 15 (same as |
| | 14578 | DH | | | above) |
| 13 | Paenisporosarcina sp. | Glu/Leu/Phe/Val | WP_017378728.1 | 64.66% | 16 (same as |
| | TG-14 | DH | | | above) |
| 14 | Rhodococcus jostii | Glu/Leu/Phe/Val | WP_073367917.1 | — | 17 (nucleotide |
| | | DH | | | sequence) |
| | | | | | 18 (amino acid |
| | | | | | sequence) |
| 15 | Rhodococcus opacus | Glu/Leu/Phe/Val | WP_128640835.1 | 95.13% | 19 (amino acid |
| | | DH | | | sequence) |
| 16 | Nocardia araoensis | Glu/Leu/Phe/Val | WP_039797578.1 | 75.64% | 20 (same as |
| | | DH | | | above) |
| 17 | Mycobacterium | Glu/Leu/Phe/Val | WP_065013575.1 | 73.43% | 21 (same as |
| | kyorinense | DH | | | above) |
| 18 | Rhodococcus | Glu/Leu/Phe/Val | WP_019746163.1 | 71.39% | 22 (same as |
| | MULTISPECIES | DH | | | above) |
| 19 | Rhodococcus sp. | pheDH | Q59771.2 | 70.75% | 23 (nucleotide |
| | | | | | sequence) |
| | | | | | 24 (amino acid |
| | | | | | sequence) |
| 20 | Lysinibacillus | Glu/Leu/Phe/Val | WP_024361159.1 | — | 25 (nucleotide |
| | sphaericus | DH | | | sequence) |
| | | | | | 26 (amino acid |
| | | | | | sequence) |
| 21 | Bacillus velezensis | Glu/Leu/Phe/Val | WP_173479019.1 | 98.08% | 27 (amino acid |
| | | DH | | | sequence) |
| 22 | Ureibacillus sp. Re31 | BCAA DH | WP_191706636.1 | 87.91% | 28 (same as |
| | | | | | above) |
| 23 | Sporosarcina sp. EUR3 | BCAA DH | WP_024534568.1 | 84.07% | 29 (same as |
| | 2.2.2 | | | | above) |
| 24 | Viridibacillus | BCAA DH | WP_038184894.1 | 83.52% | 30 (same as |
| | MULTISPECIES | | | | above) |
| 25 | Thermoactinomyces | aaDH | WP_181731632.1 | — | 31 (nucleotide |
| | intermedius | | | | sequence) |
| | | | | | 32 (amino acid |
| | | | | | sequence) |

*Sequence identities in Nos. 2 to 7: sequence identities with respect to sequence of No. 1
Sequence identities in Nos. 9 to 13: sequence identities with respect to sequence of No. 8
Sequence identities in Nos. 15 to 19: sequence identities with respect to sequence of No. 14
Sequence identities in Nos. 21 to 24: sequence identities with respect to sequence of No. 20

In addition, in a specific embodiment, an amino acid dehydrogenase derived from Lysinibacillus sphaericus, Sporosarcina ureae, Rhodococcus jostii or Thermoactinomyces intermedius, or a mutant thereof can be preferably used, and an amino acid dehydrogenase including an amino acid sequence shown in SEQ ID No. 2, 10, 18, 26 or 32 or a mutant amino acid dehydrogenase thereof can be more preferably used. However, the amino acid dehydrogenase or the mutant amino acid dehydrogenase thereof shall have catalytic activity for the reaction according to the reaction formula (IX-1) or (IX-2) or shall have enzyme activity defined in EC1.4.1.9.

In addition, in an embodiment using a mutant type bacterial amino acid dehydrogenase, a mutant type amino acid dehydrogenase having at least one of the amino acid substitutions of the following (a), (b) and (c) based on the amino acid sequence shown in SEQ ID No. 2 can be used. (a) an amino acid substitution of an amino acid corresponding to the 51st leucine (L) with a predetermined amino acid (with the proviso that the amino acid after substitution is not leucine (L), and substitution with lysine (K) is preferable);
(b) an amino acid substitution of an amino acid corresponding to the 124th glycine (G) with a predetermined amino acid (with the proviso that the amino acid after substitution is not glycine
(G), and substitution with serine(S) is preferable);
(c) an amino acid substitution of an amino acid corresponding to the 310th valine (V) with a predetermined amino acid (with the proviso that the amino acid after substitution is not valine (V), substitution with alanine (A), serine(S) or glycine (G) is preferable, and substitution with alanine (A) or glycine (G) is more preferable).

In addition, in a preferable embodiment, the mutant type amino acid dehydrogenase has at least either or both of the amino acid substitutions set forth in (b) and (c) above. This is because it becomes possible to suppress secondary production of valine relative to production of leucine when the amino acid dehydrogenase has such amino acid substitutions.

In another embodiment, a mutant type amino acid dehydrogenase having at least one of the amino acid substitutions of the following (d), (e), (f) and (g) based on the amino acid sequence shown in SEQ ID No. 26 can be used.
(d) an amino acid substitution of an amino acid corresponding to the 40th leucine (L) with a predetermined amino acid (with the proviso that the amino acid after substitution is not leucine (L), and substitution of with lysine (K) is preferable);
(e) an amino acid substitution of an amino acid corresponding to the 113th alanine (A) with a predetermined amino acid (with the proviso that the amino acid after substitution is not alanine

(A), and substitution with glycine (G) is preferable);
(f) an amino acid substitution of an amino acid corresponding to the 291st valine (V) with a predetermined amino acid (with the proviso that the amino acid after substitution is not valine (V), and substitution with leucine (L) is preferable); and
(g) an amino acid substitution of an amino acid corresponding to the 294th valine (V) with a predetermined amino acid (with the proviso that the amino acid after substitution is not valine (V), substitution with alanine (A), serine(S) or glycine (G) is preferable, and substitution with serine (S) is more preferable).

However, in each of (a) to (g) above, the amino acid before substitution and the amino acid after substitution shall be different from each other, and the mutant amino acid dehydrogenase shall have catalytic activity for the reaction according to the reaction formula (IX-1) or (IX-2) or shall have enzyme activity defined in EC1.4.1.9.

In addition, in a preferable embodiment, the mutant amino acid dehydrogenase may have all of the amino acid substitutions set forth in (a) to (g) above. This is because it becomes possible to suppress secondary production of valine relative to production of leucine when the amino acid dehydrogenase has such amino acid substitutions.

In addition, the meaning of the “amino acid corresponding to . . . ” in (a) to (g) above is to specify, based on the amino acid residues contained in the amino acid sequence shown in SEQ ID No. 2 or 26, sites to be substituted in an amino acid sequence of an amino acid dehydrogenase that serves as a target for introduction of the mutation(s). That is, the “amino acid corresponding to . . . ” in each of (a) to (g) above refers to an amino acid residue that will be aligned one-to-one with the amino acid shown in each of (a) to (g) when one-to-one alignment (pairwise alignment) is performed on the amino acid sequence of the amino acid dehydrogenase that serves as a target for introduction of the mutation(s), with respect to the amino acid sequence shown in SEQ ID No. 2 or 26, based on the identity of the former amino acid sequence to the latter amino acid sequence, by way of a technique such as ClustalW and ClustalX (Bioinformatics, Vol. 23, Issue 21, 2008 Nov., pp. 2947-2948; Bioinformatics, Volume 23, Issue 21, 1 Nov. 2007, pp 2947-2948).

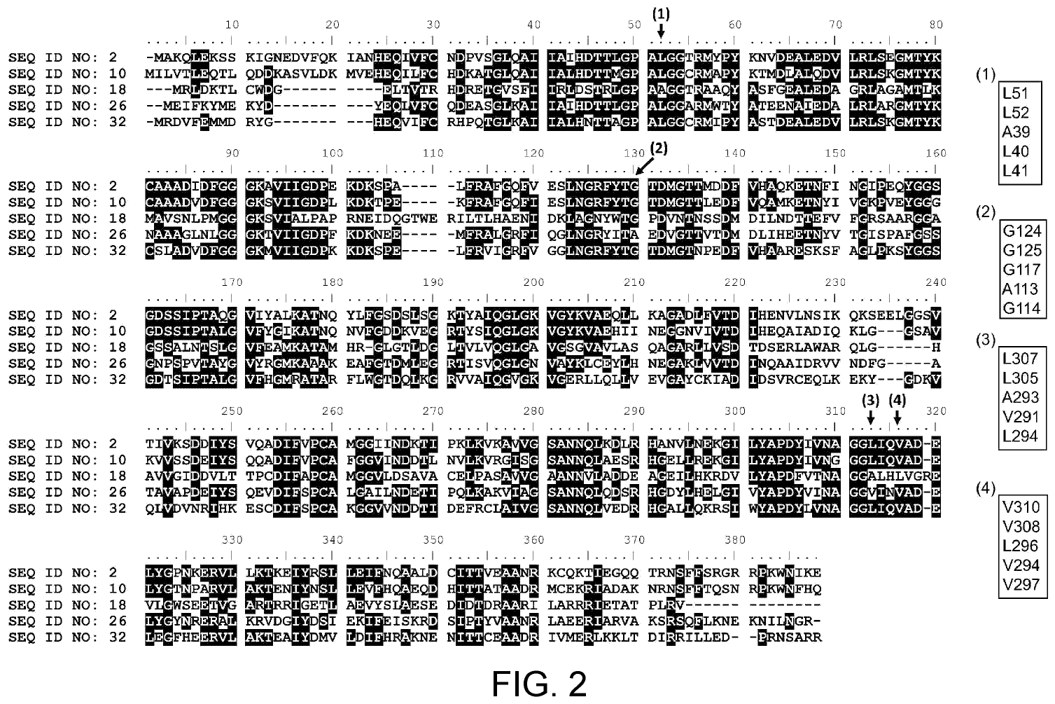

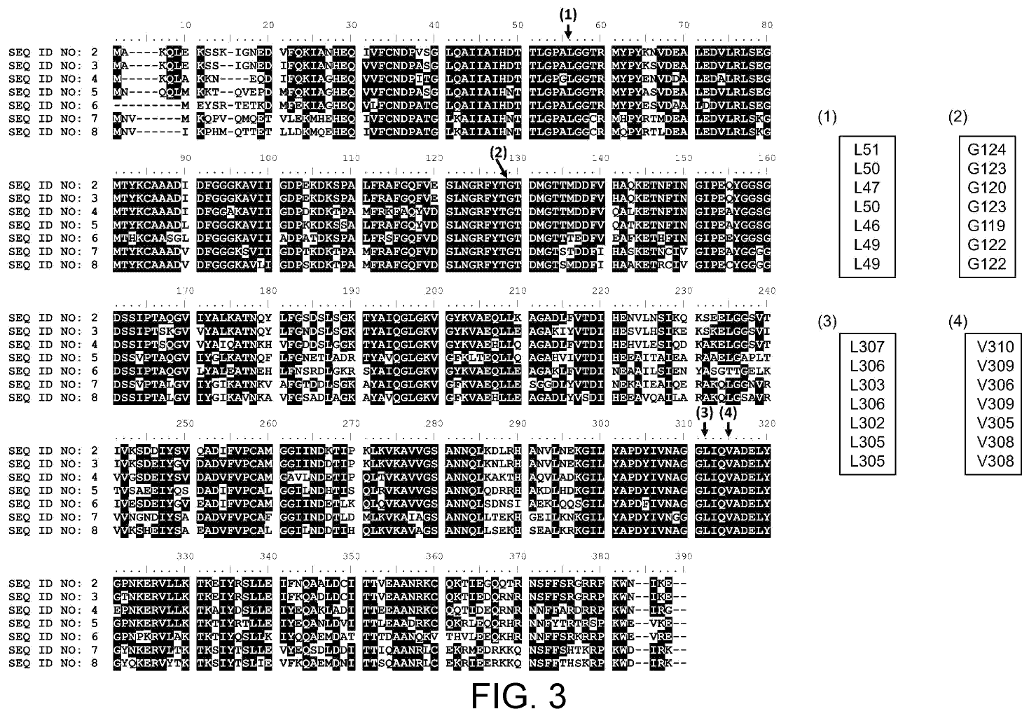

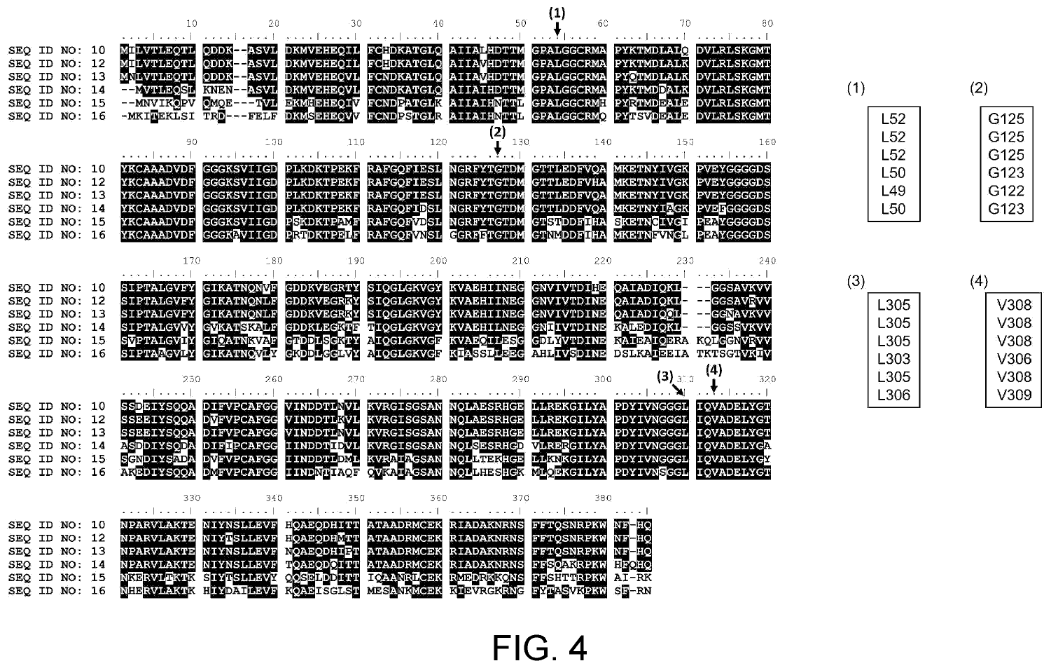

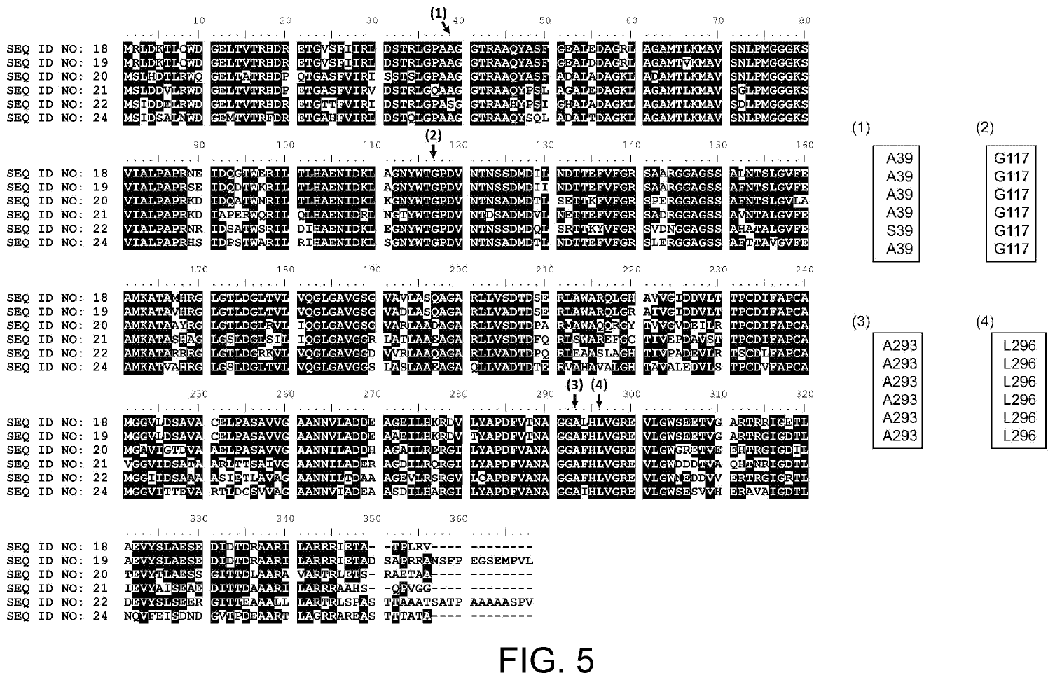

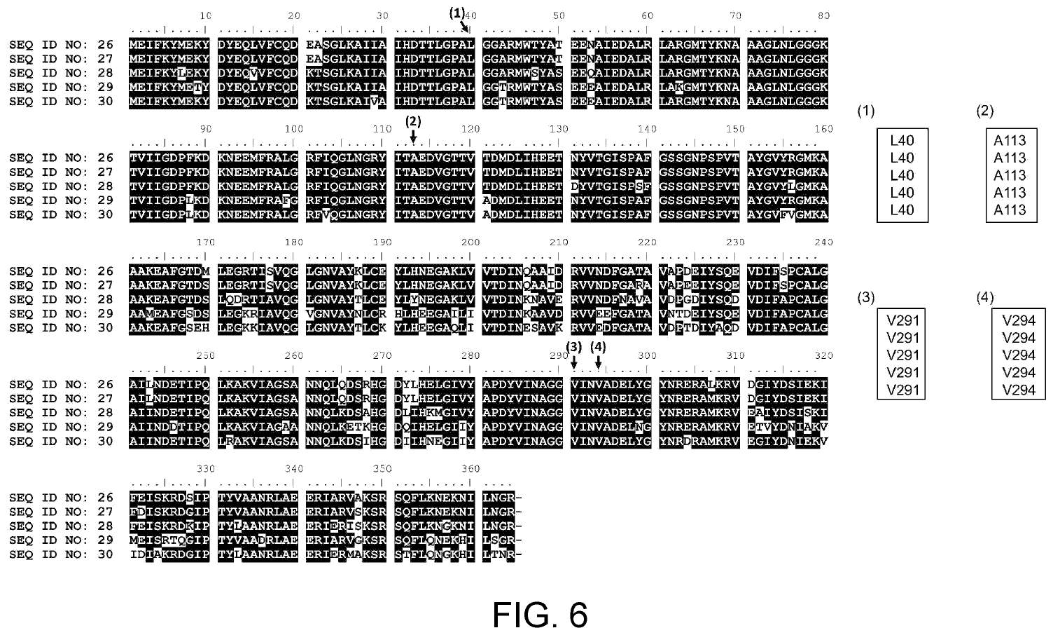

For example, FIG. 2 shows the results obtained by performing one-to-one alignment (pairwise alignment) based on ClustalW with respect to amino acid sequences of a phenylalanine dehydrogenase (SEQ ID No. 2) possessed by Lysinibacillus sphaericus, an amino acid dehydrogenase (SEQ ID No. 10) possessed by Sporosarcina ureae, a Glu/Leu/Phe/Val dehydrogenase (SEQ ID No. 18) possessed by Rhodococcus jostii, and a Glu/Leu/Phe/Val dehydrogenase (SEQ ID No. 26) possessed by Lysinibacillus sphaericus. In addition, FIGS. 3, 4, 5 and 6 show the results obtained by performing pairwise alignment based on ClustalW with respect to amino acid sequences of various bacterial amino acid dehydrogenases each having relatively high sequence identities (SEQ ID No. 2 vs. SEQ ID Nos. 3 to 8, SEQ ID No. 10 vs. SEQ ID Nos. 12 to 16, SEQ ID No. 18 vs. SEQ ID Nos. 19 to 22 and 24, and SEQ ID No. 26 vs. SEQ ID Nos 27 to 30) with respect to the respective amino acid sequences of various amino acid dehydrogenases (SEQ ID Nos. 2, 10, 18 and 26) shown in FIG. 1.

Additionally, the identities (%) of the amino acid sequences of the various amino acid dehydrogenases with respect to the amino acid sequences of SEQ ID Nos. 2, 10, 18 and 26, which are employed as reference standards for comparison in FIGS. 3 to 6, are shown in Table 2. In addition, by reference to the pairwise alignments shown in FIGS. 2 to 6, amino acid substitution positions defined in (a) to (g) above can be identified in the respective amino acid sequences shown in SEQ ID Nos. 2 to 8, 10 to 14, 16 to 20, and SEQ ID Nos. 26 to 26. That is, in FIGS. 2 to 6, groups of amino acids shown in (1) to (4) can be identified as the “amino acids corresponding to . . . ” in (a) to (g) above.

Furthermore, in some embodiments, a mutant type amino acid dehydrogenase having at least one of the amino acid substitutions of (a), (b) and (c) above relative to an amino acid dehydrogenase including the amino acid sequence shown in any one of SEQ ID Nos. 2 to 8, and having catalytic activity for the reaction according to the reaction formula (IX-1) or (IX-2) or having enzyme activity defined in EC1.4.1.9 may be employed. In addition, in another embodiment, a mutant type amino acid dehydrogenase having at least one of the amino acid substitutions of (d), (e), (f) and (g) above relative to an amino acid dehydrogenase including the amino acid sequence shown in any one of SEQ ID Nos. 2 to 8, and having catalytic activity for the reaction according to the reaction formula (IX-1) or (IX-2) or having enzyme activity defined in EC1.4.1.9 may be employed.

Furthermore, in a preferable embodiment, the amino acid dehydrogenase consists of an amino acid sequence shown in any one of the following (A) to (C) and has catalytic activity for the reaction according to the reaction formula (IX-1) or (IX-2) or has enzyme activity defined in EC1.4.1.9.
(A) an amino acid sequence shown in any one of SEQ ID Nos. 2 to 8, 10, 12 to 16, 18 to 22, 24, 26 to 30 and 32 (preferably SEQ ID No. 2, 10, 18 or 26);
(B) an amino acid sequence having a deletion, substitution and/or addition of one or more amino acids relative to the amino acid sequence set forth in (A) above; and
(C) an amino acid sequence having at least 60% sequence identity to the amino acid sequence set forth in (A) above.

In addition, in another preferable embodiment, the amino acid dehydrogenase consists of an amino acid sequence shown in any one of the following (D) to (F) and has catalytic activity for the reaction according to the reaction formula (IX-1) or (IX-2) or has enzyme activity defined in EC1.4.1.9.
(D) an amino acid sequence having at least one of the amino acid substitutions of (a), (b) and (c) above based on the amino acid sequence shown in SEQ ID No. 2 relative to the amino acid sequence shown in any one of SEQ ID Nos. 2 to 8, 10, 12 to 16, 18 to 22, 24, 26 to 30 and 32 (preferably SEQ ID Nos. 2 to 8), or an amino acid sequence having at least one of the amino acid substitutions of (d), (e), (f) and (g) above based on the amino acid sequence shown in SEQ ID No. 26 relative to the amino acid sequence shown in any one of SEQ ID Nos. 2 to 8, 10, 12 to 16, 18 to 22, 24, 26 to 30 and 32 (preferably SEQ ID Nos. 26 to 30);
(E) an amino acid sequence having a deletion, substitution and/or addition of one or more amino acids relative to the amino acid sequence set forth in (D) above; and
(F) an amino acid sequence having at least 60% sequence identity to the amino acid sequence set forth in (D) above,
   with the proviso that, in the amino acid sequences set forth in (E) and (F) above. respectively, the amino acid substitution(s) set forth in (D) above are maintained.

As used herein, a range for the “one or more” may be, for example, 1 to 100, 1 to 50, 1 to 30, preferably at least 2 or more, 2 to 20, more preferably 2 to 10, still more preferably 2 to 5. particularly preferably 2 to 4, 2 to 3, e.g., 2.

In addition, embodiments in which the term “at least 60%” in each of (C) and (F) above is replaced with preferably at least 65% or at least 70%, more preferably at least 75%, still more preferably 80%, or yet more preferably at least 85%, 86%, 87%, 88%, 89%, 90%, 91%, 92%, 93%, 94%, 95%, 96%, 97%, 98% or 99% can be employed.

Additionally, in the present application, for example, “the 310th valine (V)” may be expressed as “V310” using a one-letter code for the amino acid, “substitution of the 310th valine (V) with alanine (A)” may be expressed as “V310A,” and these corresponding terms are synonymous to each other. Other amino acids and amino acid substitutions may be expressed in the same manner.

Furthermore, in a specific embodiment, the L-leucine biosynthesis pathway includes, besides the reaction according to (ix) above, at least one of metabolic paths according to reactions (inclusive concepts) or reaction formulae (specific embodiments) shown in (i), (ii), (iii), (iv), (v). (vi), (vii) and (viii) of Tables 3.

TABLE 3

| | Reaction | | | | |
|---|---|---|---|---|---|
| Reaction | (inclusive | Reaction formula | | | Gene |
| path No. | concept) | (specific embodiment) | EC No. | Enzyme name | example |
| (i) | Production of (S)- | H⁺ + 2-pyruvic acid [symbol] (S)-2-acetolactic | 2.2.1.6 | pyruvate:pyruvate | ilvBN, alsS |
| | 2-acetolactic acid | acid + CO₂ | | acetaldehydetransferase | ilvGM |
| | from pyruvic acid | | | (decarboxylating) | ilvIH |
| | | | | | ILV2 |
| (ii) | Production of | (S)-2-acetolactic acid+ NADPH + H⁺ + | 1.1.1.86 | 2,3-Dihydroxy-3- | ilvC |
| | (R)-2,3- | (R)-2,3-dihydroxy-3-methylbutanoic | | methylbutanoate:NADP+ | ILV5 |
| | dihydroxy-3- | acid + NADP⁺ | | oxidoreductase | AHRI |
| | methylbutanoic | | | (isomerizing) | |
| | acid from (S)-2- | | | | |
| | acetolactic acid | | | | |
| (iii) | Production of 3- | (R)-2,3-dihydroxy-3-methylbutanoic | 4.2.1.9 | (R)-2,3-Dihydroxy-3- | ilvD |
| | methyl-2- | acid [symbol] 3-methyl-2-oxobutanoic acid + | | methylpentanoate hydro- | ILV3 |
| | oxobutanoic acid | H₂O | | lyase | DHAD |
| | from (R)-2,3- | | | | |
| | dihydroxy-3- | | | | |
| | methylbutanoic | | | | |
| | acid | | | | |
| (iv) | Production of | acetyl-CoA + 3-methyl-2-oxobutanoic | 2.3.3.13 | 2-isopropylmalate | leuA |
| | (2S)-2- | acid + H2O [symbol] (2S)-2-isopropylmalic | | synthase; acetyl-CoA:3- | LEU4 |
| | isopropylmalic | acid + CoA | | methyl-2-oxobutanoate | LEU9 |
| | acid from 3- | | | C-acetyltransferase | IPMS1 |
| | methyl-2- | | | (thioester-hydrolysing, | IPMS2 |
| | oxobutanoic acid | | | carboxymethyl-forming) | |
| (v) | Production of 2- | (2S)-2-isopropylmalic acid [symbol] 2- | 4.2.1.33 | 2-Isopropylmalate hydro- | leuC |
| | isopropylmalic | isopropylmalic acid + H₂O | | lyase | leuD |
| | acid from (2S)-2- | | | | SSU1-3 |
| | isopropylmalic | | | | Leu1, Leu2 |
| | acid | | | | IIL1 |
| (vi) | Production of | 2-isopropylmalic acid + H₂O [symbol] | 4.2.1.33 | 3-Isopropylmalate hydro- | |
| | (2R,3S)-3- | (2R,3S)-3-isopropylmalic acid | | lyase | |
| | isopropylmalic | | | | |
| | acid from 2- | | | | |
| | isopropylmalic | | | | |
| | acid | | | | |
| (vii) | Production of | (2R,3S)-3-isopropylmalic acid+ NAD⁺ | 1.1.1.85 | (2R,3S)-3- | leuB |
| | (2S)-2-isopropyl- | [symbol] (2S)-2-isopropyl-3-oxosuccinic acid + | | isopropylmalate:NAD+ | IMDH1-3 |
| | 3-oxosuccinic | NADH + H⁺ | | oxidoreductase | LEU1, LEU2 |
| | acid from | | | | |
| | (2R,3S)-3- | | | | |
| | isopropylmalic | | | | |
| | acid | | | | |
| (viii) | Production of 4- | (2S)-2-isopropyl-3-oxosuccinic acid [symbol] | 1.1.1.85 | | |
| | methyl-2- | 4-methyl-2-oxopentanoic acid + CO₂ | (spontaneous) | | |
| | oxopentanoic | | | | |
| | acid from (2S)-2- | | | | |
| | isopropyl-3- | | | | |
| | oxosuccinic acid | | | | |

Furthermore, in some embodiments, the L-leucine biosynthesis pathway includes an intermediate metabolic path according to reactions (inclusive concepts) or reaction formulae (specific embodiments) shown in (iia) and (iib) of Table 4 in addition to or in place of the reaction (inclusive concept) or reaction formula (specific embodiment) shown in (ii) above.

TABLE 4

| Reaction | Reaction (inclusive | Reaction formula | EC | | Gene |
|---|---|---|---|---|---|
| path No. | concept) | (specific embodiment) | No. | Enzyme name | (example) |
| (iia) | Production of 3-hydroxy-3- | (S)-2-acetolactic acid [symbol] 3- | 5.4.99.3 | (S)-2-Acetolactate | ilvC |
| | methyl-2-oxobutanoic acid | hydroxy-3-methyl-2- | | methylmutase | |
| | from (S)-2-acetolactic acid | oxobutanoic acid | 1.1.1.86 | | |
| (iib) | Production of (R)-2,3- | 3-hydroxy-3-methyl-2- | 1.1.1.86 | (R)-2,3-Dihydroxy-3- | ilvC |
| | dihydroxy-3-methylbutane | oxobutanoic acid+ NADPH + | | methylpentanoate:NADP+ | ilv-2 |
| | carbonic acid from 3- | H⁺ [symbol] (R)-2,3-dihydroxy-3- | | oxidoreductase (isomerizing) | ILV5 |
| | hydroxy-3-methyl-2- | methylbutane carbonic acid + | | | AHRI |
| | oxobutanoic acid | NADP⁺ | | | |

Furthermore, in a specific embodiment, the L-leucine biosynthesis pathway at least includes a metabolic path according to the reaction (inclusive concept) or the reaction formula (specific embodiment) shown in (iii) of Table 3 in addition to the reaction (inclusive concept) or the reaction formulae (specific embodiments) shown in (ix) of Table 1. In addition, in a preferable embodiment, the L-leucine biosynthesis pathway may include all of metabolic paths according to the reactions (inclusive concepts) or the reaction formulae (specific embodiments) shown in (i), and (ii) [(iia) and (iib) of Table 4 in addition to or in place of (ii)] of Table 3 and the reactions (inclusive concepts) or the reaction formulae (specific embodiments) shown in (iii), (iv), (v), (vi), (vii) and (viii) of Table 3, in addition to the reaction (inclusive concept) or the reaction formulae (specific embodiments) shown in (ix) of Table 1.

Meanwhile, as described above, the amino acid dehydrogenase would have catalytic activity for the reaction according to the reaction formula (IX-1) or (IX-2) shown in (ix) above or have enzyme activity defined in EC1.4.1.9. In this respect, some of various amino acid dehydrogenases having enzyme activity defined in EC1.4.1.9 may also catalyze the reaction ((x) of Table 5) that secondarily produces L-valine from 3-methyl-2-oxobutanoic acid produced according to the reaction shown in (iii) above (Tables 1 and 5, and FIG. 1). In addition, as shown in Table 5, production of L-valine from 3-methyl-2-oxobutanoic acid may be caused also according to reactions catalyzed by enzymes specified in EC1.4.1.8, EC1.4.1.23, EC2.6.1.42 and EC2.6.1.66, respectively.

TABLE 5

| | Reaction | | | | Gene |
|---|---|---|---|---|---|
| Reaction | (inclusive | Reaction formula | EC | | (exam- |
| path No. | concept) | (specific embodiment) | number | Enzyme name | ple) |
| (x) | Secondary | 3-methyl-2-oxobutanoic acid + NH₃ + | 1.4.1.9 | leucine dehydrogenase | ldh |
| | production of | NADH + H⁺ [symbol] L-valine + H₂O + NAD⁺ | | (side reaction) | bcd |
| | L-valine from | 3-methyl-2-oxobutanoic acid + NH₃ + | 1.4.1.8 | valine dehydrogenase | vdh |
| | 3-methyl-2- | NADPH + H⁺ [symbol] L-valine + H₂O + NADP⁺ | | (NADP⁺) | |
| | oxobutanoic | 3-methyl-2-oxobutanoic acid + NH₃ + | 1.4.1.23 | valine dehydrogenase | vdh |
| | acid | NADH + H⁺ [symbol] L-valine + H₂O + NAD⁺ | | (NAD⁺) | |
| | | 3-methyl-2-oxobutanoic acid + L-glutamic | 2.6.1.42 | L-Valine:2- | ilvE |
| | | acid [symbol] L-valine + 2-oxoglutaric Acid | | oxoglutarate | BAT2 |
| | | | | aminotransferase | eca39 |
| | | 3-methyl-2-oxobutanoic | 2.6.1.66 | valine—pyruvate | avtA |
| | | acid + L-alanine [symbol] | | aminotransferase | aspB |
| | | L-valine + pyruvic acid | | | alaA |

Therefore, in an embodiment in which the L-leucine biosynthesis pathway includes at least the reaction shown in (iii) above in addition to the leucine production reaction shown in (ix) above, one having enzyme activity that exhibits reduced secondary production of L-valine (EC1.4.1.9) according to (x) above, while causing the leucine production reaction (EC1.4.1.9) according to (ix) above to dominantly proceed, may preferably be selected for the amino acid dehydrogenase (EC1.4.1.9) capable of catalyzing the leucine production reaction according to (ix) above. For the amino acid dehydrogenase having such properties, for example, an amino acid dehydrogenase consisting of an amino acid sequence shown in any one of the following (G1) to (H), and having enzyme activity that causes an increase in a production ratio of L-leucine/L-valine and/or L-leucine/L-isoleucine in the recombinant microorganism owing to expression of the amino acid dehydrogenase in cells of the recombinant microorganism, compared to a control microorganism, may preferably be employed. This is because it is desirable to reduce the secondary production of L-valine and L-leucine in L-leucine production since branched-chain amino acids L-leucine, L-valine, and L-leucine have very similar properties and chemical behaviors, and therefore, are difficult to separate from each other.
(G1) an amino acid sequence shown in SEQ ID No. 18 (the amino acid dehydrogenase according to No. 14 in Table 2);
(G2) an amino acid sequence having an amino acid substitution of V310A or V310C relative to the amino acid sequence shown in SEQ ID No. 2 (relative to the amino acid dehydrogenase according to No. 1 in Table 2);
(G3) an amino acid sequence having a combination of amino acid substitutions of G124S and V310A or V310G relative to the amino acid sequence shown in SEQ ID No. 2;
(G4) an amino acid sequence having a combination of amino acid substitutions of L40K. A113G, V291L, and V294S relative to the amino acid sequence shown in SEQ ID No. 26 (the amino acid dehydrogenase according to No. 20 in Table 2);
(H) an amino acid sequence having a deletion, substitution and/or addition of one or more amino acids relative to the amino acid sequence set forth in any one of (G1) to (G4) above,
   with the proviso that, in (H) above, the amino acid substitutions set forth in (G2) to (G4) above are maintained.

In addition, in (H) above, a range for the “one or more” is, for example, 1 to 100, 1 to 50, 1 to 30, preferably at least 2 or more, 2 to 20, more preferably 2 to 10, still more preferably 2 to 5, particularly preferably 2 to 4, 2 to 3, for example, 2.

Additionally, for confirming an increase in the production ratio of L-leucine/L-valine and/or L-leucine/L-isoleucine, a negative control strain which corresponds to the strain type of the recombinant microorganism and into which a gene encoding the amino acid dehydrogenase has not been introduced or in which expression of the amino acid dehydrogenase has not been enhanced can be employed as the control microorganism. In addition, for the production ratios of L-leucine/L-valine and/or L-leucine/L-isoleucine, steps for production of L-leucine using the recombinant microorganisms and the negative control strain, respectively, would be performed, production amounts of L-leucine, and L-valine or L-isoleucine would be then determined for collected samples (for example, cultures, supernatants of cultures, and isolated strains), and thus, production ratios of L-leucine/L-valine and/or L-leucine/L-isoleucine can be calculated from values of such determined production amounts. For example, as in examples below, since produced L-leucine and L-valine or L-isoleucine would be present in culture media, the concentrations (mM) of L-leucine and L-valine or L-isoleucine in supernatants of the culture media that have been separated from the culture products may be analyzed based on the HPLC technique, and thus, production ratios of L-leucine/L-valine and/or L-leucine/L-isoleucine may be calculated based on the values of the concentrations. However, for the method for determining the production ratios of L-leucine/L-valine and/or L-leucine/L-isoleucine, any methods can be employed without any particular limitations as long as they can reasonably confirm increases in the production ratios of L-leucine/L-valine and/or L-leucine/L-isoleucine.

In addition, in a specific embodiment, it may be preferable to employ an amino acid dehydrogenase that, when a gene therefor is introduced into a predetermined host in a forcibly expressible manner, realizes, for the production ratio of L-leucine/L-valine, a value of 1.64 or more, 1.66 or more, 1.68 or more, 1.70 or more, 1.72 or more, 1.74 or more, 1.76 or more, 1.78 or more, 1.80 or more, 1.82 or more, 1.84 or more, 1.86 or more, 1.88 or more, 1.90 or more, 1.92 or more, 1.94 or more, 1.96 or more, 1.98 or more, 2.00 or more, 2.02 or more, 2.04 or more, 2.06 or more, 2.08 or more, 2.10 or more, 2.12 or more, 2.14 or more, 2.16 or more, 2.18 or more, 2.20 or more, 2.22 or more, 2.24 or more, 2.26 or more, 2.28 or more, 2.30 or more, 2.32 or more, 2.34 or more. 2.36 or more, 2.38 or more, 2.40 or more, 2.41 or more, 2.42 or more, 2.43 or more, 2.44 or more. 2.45 or more, 2.46 or more, 2.47 or more, 2.48 or more, 2.49 or more, 2.50 or more, 2.51 or more, 2.52 or more, 2.53 or more, 2.54 or more, 2.55 or more, 2.56 or more, 2.57 or more, 2.58 or more. 2.59 or more. 2.60 or more, 2.61 or more, 2.62 or more, 2.63 or more, 2.64 or more, 2.65 or more, 2.66 or more, 2.67 or more, 2.68 or more, 2.69 or more, 2.70 or more, 2.71 or more, 2.72 or more, 2.73 or more, 2.74 or more, 2.75 or more, 2.76 or more, 2.77 or more, 2.78 or more, 2.79 or more, 2.80 or more, 2.81 or more, 2.82 or more, 2.83 or more, 2.84 or more, 2.85 or more, 2.86 or more, 2.87 or more, 2.88 or more, 2.89 or more, 2.90 or more, 2.91 or more, 2.92 or more, 2.93 or more, 2.94 or more, 2.95 or more, 2.96 or more, 2.97 or more, 2.98 or more, 2.99 or more, or 3.00 or more.

Furthermore, in a specific embodiment, it may be preferable to employ an amino acid dehydrogenase that, when a gene therefor is introduced into a predetermined host in a forcibly expressible manner, realizes, for the production ratio of L-leucine/L-isoleucine, a value of 15.0 or more, 20.0 or more, 25.0 or more, 29.0 or more, 30.0 or more, 31.0 or more, 32.0 or more, 33.0 or more, 34.0 or more, 35.0 or more, 36.0 or more, 37.0 or more, 38.0 or more, 39.0 or more, 40.0 or more, 41.0 or more, 42.0 or more, 43.0 or more, 44.0 or more, 45.0 or more, 50.0 or more, 55.0 or more, 60.0 or more, 65.0 or more, 70.0 or more, 75.0 or more, 76.0 or more, 77.0 or more, 78.0 or more, 79.0 or more, 80.0 or more, 81.0 or more, 82.0 or more, 83.0 or more, 84.0 or more, 85.0 or more, 86.0 or more, 90.0 or more, 95.0 or more, 100.0 or more, 105.0 or more, 110.0 or more, 115.0 or more, 120.0 or more, 125.0 or more, 130.0 or more, 135.0 or more, 140.0 or more, 145.0 or more, 150.0 or more, 155.0 or more, 160.0 or more, 165.0 or more, 170.0 or more, 175.0 or more, 180.0 or more, 185.0 or more, 190.0 or more, 195.0 or more, 200.0 or more, 205.0 or more, 215.0 or more, 220.0 or more, 225.0 or more, 230.0 or more, 235.0 or more, 240.0 or more, 245.0 or more, or 250.0 or more.

Furthermore, in a particularly preferable embodiment, an amino acid dehydrogenase having the amino acid sequence shown in any one of (G1) to (G4) above and having activity that exhibits reduced production of L-valine from 3-methyl-2-oxobutanoic acid, while causing production of L-leucine from 4-methyl-2-oxopentanoic acid to dominantly proceed, may be employed for the amino acid dehydrogenase (EC1.4.1.9) that can catalyze the leucine production reaction according to (ix) above. Such an amino acid dehydrogenase may be preferably employed because, as shown in examples, the amino acid dehydrogenase makes it possible to reliably reduce the secondary production of L-valine from 3-methyl-2-oxobutanoic acid, thus efficiently producing L-leucine from 4-methyl-2-oxopentanoic acid.

In addition, since the secondary production of L-valine from 3-methyl-2-oxobutanoic acid in (x) above would be caused through enzyme reactions defined in EC1.4.1.8, EC1.4.1.23, EC2.6.1.42 and EC2.6.1.66, respectively, an embodiment in which activity of enzymes that catalyze at least one of the enzyme reactions is reduced or inactivated may also be preferably adopted.

In addition, in another embodiment, when the wild type amino acid dehydrogenase according to EC1.4.1.9 in the host causes the secondary production of L-valine in (x) above to considerably proceed, relative to the production of leucine shown in (ix) above, a recombinant microorganism that has been modified in a manner that the recombinant microorganism is capable of expressing in a forcible mode an amino acid dehydrogenase having activity that exhibits reduced production of L-valine from 3-methyl-2-oxobutanoic acid and causes production of L-leucine from 4-methyl-2-oxopentanoic acid to dominantly proceed, while the activity of the wild type amino acid dehydrogenase enzyme in the host has been reduced or inactivated, may also be employed.

For a specific embodiment, when a bacterium (preferably a coryneform bacterium, more preferably a bacterium belonging to the genus Corynebacterium, and still more preferably Corynebacterium glutamicum) is used as a host, a recombinant microorganism having the following modifications (i), (iv), (x) and (ix) can preferably be employed.
(i) enhancing enzyme activity defined in EC2.2.1.6;
(iv) enhancing enzyme activity defined in EC2.3.3.13;
(x) reducing or inactivating enzyme activity defined in EC2.6.1.42;
(ix) enhancing enzyme activity for catalyzing the reaction according to the reaction formula (IX-1) or (IX-2) or enhancing enzyme activity defined in EC1.4.1.9.

In that case, in some embodiments, the modification (ix) may be realized by introducing a gene encoding an amino acid dehydrogenase into a bacterium serving as the host in a forcibly expressible manner, in which the amino acid dehydrogenase exhibits catalytic activity for the reaction according to the reaction formula (IX-1) or (IX-2) or enzyme activity defined in EC1.4.1.9, and further exhibits in the bacterium a value equivalent to or even higher than any of the above-described values from 1.64 to 3.00 for the production ratio of L-leucine/L-valine.

Meanwhile, the enzyme activity defined in EC1.4.1.9 in (ix) above may preferably be enzyme activity that exhibits reduced production of L-valine from 3-methyl-2-oxobutanoic acid, while causing generation of L-leucine from 4-methyl-2-oxopentanoic acid to dominantly proceed, and as an example of the amino acid dehydrogenase that can realize such enzyme activity, an amino acid dehydrogenase including an amino acid sequence shown in any one of (G1) to (H) above and having activity that exhibits reduced production of L-valine from 3-methyl-2-oxobutanoic acid and causes production of L-leucine from 4-methyl-2-oxopentanoic acid to dominantly proceed may be mentioned.

In a more preferable embodiment, when a coryneform bacterium is used as a host, for the recombinant microorganism of the present invention, a recombinant coryneform bacterium that satisfies at least one of the following conditions (i), (iv) and (x), and the condition (ix) can be employed.
(i) a bacterial ilvBN gene, an ortholog thereof, or a mutant type gene of any of these has been introduced thereinto in a forcibly expressible manner;
(iv) a bacterial leuA gene, an ortholog thereof, or a mutant type gene of any of these has been introduced in a forcibly expressible manner;
(x) a (wild type) ilvE gene or an ortholog thereof present on the genome of the host has been inactivated or deficient;
(ix) a gene encoding any of the above predetermined amino acid dehydrogenases in the present invention has been introduced thereinto in a forcibly expressible manner.

In this case, in some embodiments, the modification (ix) may be realized by introducing a gene encoding an amino acid dehydrogenase into the bacterium serving as the host in a forcibly expressible manner, in which the amino acid dehydrogenase exhibits enzyme activity defined in EC1.4.1.9 and exhibits in the bacterium a value equivalent to or even higher than any of the above-described values from 1.64 to 3.00 for the production ratio of L-leucine/L-valine.

In a particularly preferable embodiment, when a bacterium belonging to the genus Corynebacterium (more preferably Corynebacterium glutamicum) is used as a host, for the recombinant microorganism of the present invention, a recombinant strain that satisfies at least one of the following conditions (i), (iv) and (x), and the condition (ix) may be adopted.
(i) a gene encoding a protein defined in the following (J1) or (J2) and a gene encoding a protein defined in any one of the following (K1) to (K4) have been introduced thereinto in a forcibly expressible manner.
(iv) a gene encoding a protein defined in any of the following (L1) to (L4) has been introduced in a forcibly expressible manner,
(x) an ilvE gene (for example, a nucleotide sequence shown in SEQ ID No. 33 or an amino acid sequence shown in SEQ ID No. 34 in Corynebacterium glutamicum strain ATCC13032) or an ortholog thereof has been inactivated or deficient;
(ix) a gene encoding the predetermined amino acid dehydrogenase of the present invention has been introduced in a forcibly expressible manner.
(J1) a protein consisting of an amino acid sequence shown in SEQ ID No. 38;
(J2) a protein consisting of an amino acid sequence having a deletion, substitution and/or addition of one or more amino acids relative to the amino acid sequence shown in SEQ ID No. 38, the protein being capable of functioning as an acetolactate synthase large subunit;
(K1) a protein consisting of an amino acid sequence shown in SEQ ID No. 40;
(K2) a protein consisting of an amino acid sequence having deletion, substitution and/or addition of one or more amino acids relative to the amino acid sequence shown in SEQ ID No. 40, the protein capable of functioning as an acetolactate synthase small subunit;
(K3) a protein consisting of an amino acid sequence having amino acid substitutions of G20D. I21D and I22F relative to the amino acid sequence shown in SEQ ID No. 40, the protein functioning as an acetolactate synthase small subunit and having resistance to feedback inhibition by at least one of valine, leucine and isoleucine;
(K4) a protein consisting of an amino acid sequence having a deletion, substitution and/or addition of one or more amino acids relative to the amino acid sequence of the protein set forth in (K3) above (with the proviso that the amino acid substitutions of G20D, I21D and I22F are maintained in the protein, and the protein functions as an acetolactate synthase small subunit and has resistance to feedback inhibition of enzyme activity by at least one of valine, leucine and isoleucine);
(L1) a protein consisting of an amino acid sequence shown in SEQ ID No. 36;
(L2) a protein consisting of an amino acid sequence having a deletion, substitution and/or addition of one or more amino acids relative to the amino acid sequence shown in SEQ ID No. 36, the protein having enzyme activity specified in EC2.3.3.13 ((iv) in Table 3);
(L3) a protein consisting of an amino acid sequence including amino acid substitutions of G530D, G532D and A535T in the amino acid sequence shown in SEQ ID No. 36, the protein having enzyme activity specified in EC2.3.3.13 ((iv) in Table 3) and having resistance to feedback inhibition of enzyme activity by L-leucine;
(L4) a protein consisting of an amino acid sequence having a deletion, substitution and/or addition of one or more amino acids relative to the amino acid sequence of the protein set forth in (L3) above (with the proviso that the amino acid substitutions of G530D, G532D and A535T are maintained in the protein, and the protein has enzyme activity specified in EC2.3.3.13 ((iv) in Table 3) and has resistance to feedback inhibition of enzyme activity by L-leucine).

In addition, the range for the “one or more” in (J2), (K2), (K4), (L2), and (L4) above may be, for example, 1 to 100, 1 to 50, 1 to 30, preferably at least 2 or more, 2 to 20, more preferably 2 to 10, still more preferably 2 to 5, particularly preferably 2 to 4, 2 to 3, e.g., 2.

Furthermore, in a specific embodiment, in the recombinant microorganism of the present invention, the activity of one or more enzymes that catalyze the reaction of the reaction path according to (xi) shown in Table 6, i.e., the “secondary production of L-isoleucine from(S)-3-methyl-2-oxopentanoic acid” may further be reduced or removed, and more specifically, the activity of one or more enzymes that catalyze either or both of the reaction formulae shown in Table 6 may be reduced or removed.

TABLE 6

| | Reaction | | | | |
|---|---|---|---|---|---|
| Reaction | (inclusive | Reaction formula | EC | | Gene |
| path No. | concept) | (specific embodiment) | number | Enzyme name | (example) |
| ( ) | Secondary | (S)-3-methyl-2-oxopentanoic acid + NH₃ + | 1.4.1.9 | leucine | ldh |
| | production of L- | NADH + H⁺ [symbol] L-isoleucine + NAD⁺ + H₂O | | dehydrogenase | bcd |
| | isoleucine from | | | (side reaction) | |
| | (S)-3-methyl-2- | (S)-3-methyl-2-oxopentanoic acid + L- | 2.6.1.42 | branched-chain- | ilvE |
| | oxopentanoic acid | glutamic aci d [symbol] L-isoleucine + 2- | | amino-acid | BAT2 |
| | | oxoglutaric Acid | | transaminase; | ASP |
| | | | | transaminase B | eca39 |

Additionally, in the present invention, the term “include (including) a gene or enzyme in an expressible form” refers to a concept encompassing not only introducing an exogenous or heterologous gene into host microorganisms in a forcibly expressible form but also utilizing the gene and its expression regulator(s) inherently possessed by the host microorganism without any modifications thereto. On the other hand, terms such as “introduce (introducing) a predetermined gene or enzyme in a forcibly expressible manner” means that an exogenous or heterologous gene is introduced into a host microorganism in a forcibly expressible manner. As for the relationship between the term “include (including) a gene or enzyme in an expressible form” and the term “introduce (introducing) a predetermined gene or enzyme in a forcibly expressible manner,” the former can encompass the latter.

In addition, the term “exogenous gene” does not exclude any genes inherently possessed by the host, and thus, encompasses a configuration in which genes inherently possessed by the host and their gene expression regulators are utilized in intact forms.

In addition, although a specific configuration of “in a forcibly expressible manner” in “a gene or enzyme is (has been) introduced in a forcibly expressible manner” is not particularly limited, examples thereof include a configuration in which a predetermined promoter is connected to a region 5′ upstream of a coding region of a target gene or enzyme, and a configuration in which a terminator is optionally connected to a region 3′ downstream of the coding region.

The promoter may be an inducible promoter or a constitutive promoter. Examples of promoters that can be used in the present invention include a trc promoter, tacI promoter, tacII promoter, T5 promoter, T7 promoter, lac promoter, trp promoter, tet promoter, EFTu promoter, groES promoter, SOD promoter, P15 promoter, IdhA promoter, gapA promoter, dapA promoter, metE promoter, and tuf promoter.

### Examples of Terminators Include TrrnB, TGroEL, Ttrp, and TT7

The recombinant microorganism according to the present invention may include in an expressible form enzymes that are each capable of catalyzing reaction paths in a leucine biosynthesis pathway so as to realize some or all of the reaction paths, thus having the leucine biosynthesis pathway, and may be a microorganism into which genes for encoding enzymes that are each capable of catalyzing some of the reaction paths have been introduced in a forcibly expressible manner. That is, in the above specific embodiments, the recombinant microorganism according to the present invention has been genetically engineered and modified so that the embodiments can be realized. In addition, genetic engineering and modification techniques used to prepare the recombinant microorganism according to the present invention are not particularly limited, any various techniques known in the field of genetic engineering and molecular biology besides techniques shown in the following examples may be used.

### <L-Leucine Production Method>

Hereinafter, steps (p) and (q) and other optional steps that can be included in the method according to the present invention will be described with reference to specific embodiments.

[Step (p)]

“Incubating a recombinant microorganism in a predetermined culture medium (X)” in step (p) means culturing or reacting cells of the recombinant microorganism according to the present invention in the predetermined culture medium (X) to produce L-leucine through an L-leucine biosynthesis pathway that the recombinant microorganism has. Needless to say, the method according to the present invention includes, as step (p), culturing the microorganism under aerobic conditions that allow substantial proliferation of the microorganism, to thereby produce L-leucine. This is because, when the recombinant microorganism having the predetermined L-leucine biosynthesis pathway is cultured under such aerobic conditions, metabolic reactions according to the L-leucine biosynthesis pathway proceed within the microbial cells with as the proliferation or growth of the recombinant microorganism, and thus, L-leucine may be produced within the microbial cells, or L-leucine produced by the recombinant microorganism may be secreted into the reaction culture medium.

On the other hand, for example, it has been known that a microorganism such as an Escherichia bacterium, e.g., E. coli, and a coryneform bacterium is capable of causing a predetermined metabolic system without substantial proliferation, in a culture medium (X) under reducing conditions. At least some of the recombinant microorganisms according to the present invention are no exception, and, in such a manner, is capable of causing a predetermined metabolic system including the L-leucine biosynthesis pathway without substantial proliferation, thereby producing L-leucine, in the culture medium (X) under reducing conditions. Therefore, in a specific embodiment. “incubating a recombinant microorganism in a predetermined culture medium (X)” in step (p) may encompasses maintaining cells of the recombinant microorganism according to the present invention in a culture medium (X) under reducing conditions that do not cause substantial proliferation of the cells, to thereby produce L-leucine.

In a preferable embodiment, in step (p), the recombinant microorganism according to the present invention is cultured under aerobic conditions to thereby produce L-leucine. This is because, in a case where a recombinant microorganism using a bacterium such as a coryneform bacterium as a host is adopted, L-leucine can be produced in excellent yield as described hereinbelow when the microorganism is cultured under aerobic conditions.

In addition, the “processed product of microbial cells thereof” in step (p) may be any product obtained by performing some kind of treatment on microbial cells of the recombinant microorganism according to the present invention, and is not particularly limited as long as it realizes step (p). In a specific embodiment, immobilized microbial cells of the recombinant microorganism according to the present invention, which have been immobilized onto a support made of acrylamide, or, for example, various polymers such as carrageenan may be used as the “processed product of microbial cells thereof” in step (p). That is, in such an embodiment, in step (p), the immobilized microbial cells of the recombinant microorganism according to the present invention, which have been immobilized onto a support, are incubated in a predetermined culture medium (X) so as to cause the L-leucine biosynthesis pathway possessed by the microbial cells to proceed and thus produce L-leucine.

Additionally, the “predetermined culture medium (X)” is not limited as long as it realizes production of L-leucine through incubation of the recombinant microorganisms according to the present invention or the processed product of microbial cells thereof therein, and it refers to a concept encompassing: for example, various culture media used for culturing and proliferating microorganisms; reaction culture media or culture media under reducing conditions that make it possible to proceed a predetermined metabolic reaction without proliferation of microorganisms; and culture media (X) in which each enzyme that can realize the leucine biosynthesis pathway exhibits desired enzyme activity, as described above.

[Step (p′)]

In addition, in some embodiments, the method according to the present invention may further include, before step (p), (p′) culturing and proliferating a recombinant microorganism under aerobic conditions in a predetermined culture medium (Y), and then recovering the recombinant microorganism from the culture medium (Y), to thereby prepare microbial cells of the recombinant microorganism or the processed product of microbial cells thereof used in step (p).

In this case, with regard to conditions for culturing the recombinant microorganism, it would be sufficient to set the conditions appropriately so that the recombinant microorganism can sufficiently proliferate, and thus, a sufficient amount of microbial cells or a processed product of the microbial cells can be obtained. Specifically, they may be cultured in an appropriate culture medium (Y) at a culture temperature of about 25° C. to 38° C. under aerobic conditions for a culture time of about 12 hours to 48 hours. In addition, for microbial cell stocks obtained by freeze-drying or frozen storage, they may be once seeded on a solid culture medium, and thus, colonies or the like, which have been confirmed to grow on the solid culture medium, may be further inoculated into the above culture medium (Y) to thereby prepare the recombinant microorganism to be used in step (p).

### [Culture Medium (Y) and Culture Medium (X)]

Hereinafter, the culture medium (Y) in step (p′) and the culture medium (X) in step (p) will be described. In the meantime, the method according to the present invention does not exclude an embodiment in which the culture medium (Y) and the culture medium (X) have the same composition or physical properties.

The culture medium (Y) and the culture medium (X) are not particularly limited, and those suitably selected depending on the type of recombinant microorganism adopted in the method of the present invention may be used. Specifically, for the culture medium (Y) and the culture medium (X), a natural culture medium or a synthetic culture medium containing a carbon source, a nitrogen source, an inorganic salt, other nutritional substances or the like may be used. Examples of ingredients contained in the culture medium include the followings.

Examples of the carbon source include carbohydrates, more specifically sugars including polysaccharides and monosaccharides, and various materials containing such sugars, and, for example, the following ingredients may be mentioned.

Monosaccharides such as glucose, fructose, mannose, xylose, arabinose, and galactose; disaccharides such as sucrose, maltose, lactose, cellobiose, xylobiose, and trehalose; polysaccharides such as cellulose, starch, glycogen, agarose, pectin, and alginic acid; molasses, etc.; non-edible agricultural wastes and non-edible biomass (resources made from non-edible herbaceous plants or woody plants) such as rice straws, forest remaining materials, bagasse, and corn stover; saccharified solutions containing a plurality of sugars such as glucose and xylose. obtained by saccharifying energy crops such as switchgrass, napier grass or miscanthus based on saccharification enzymes or the like; sugar alcohols such as mannitol, sorbitol, xylitol, and glycerin: organic acids such as acetic acid, citric fermentation, lactic acid, fumaric acid, maleic acid, and gluconic acid; alcohols such as ethanol, propanol, and butanol; and hydrocarbons such as normal paraffin.

Among these, monosaccharides are preferable, and glucose is more preferable. Additionally, sugars (e.g., disaccharides, oligosaccharides, and polysaccharides) including glucose are also preferable. In addition, for the carbon source, one kind of material can solely be used, or two or more kinds of materials may be combined. In addition, the concentration of the carbon source in the culture medium (Y) or the culture medium (X) may be preferably about 1% to 20% w/v, more preferably about 2% to 10% w/v, and still more preferably about 2% to 5% w/v. In addition, the concentration of sugars in the culture medium (X) may be, for example, about 1% to 20% w/v, more preferably about 2% to 10% w/v, and still more preferably about 2% to 5% w/v.

For the nitrogen source, inorganic or organic ammonium compounds such as ammonia, ammonium carbonate ((NH₄)₂CO₃), ammonium chloride, ammonium sulfate, ammonium nitrate, and ammonium acetate; and urea, aqueous ammonia, sodium nitrate, potassium nitrate, or the like can be used. Additionally, nitrogen-containing organic compounds such as corn steep liquor, meat extract, peptone, NZ-amine, protein hydrolysate, and amino acid can be used.

In addition, for the nitrogen source, one kind of material can solely be used, or two or more kinds of materials can be combined. The concentration of the nitrogen source in the culture medium (Y) or the culture medium (X) may be suitably adjusted depending on conditions such as the type of the recombinant microorganism used therein, the type or properties of a desired target substance, reaction conditions, and the types of nitrogen compounds, and therefore, the concentration is not particularly limited, but may be adjusted to, for example, about 0.1% to 10% w/v.

Examples of the inorganic salt include monopotassium phosphate, dipotassium phosphate, magnesium sulfate (hydrate), sodium chloride, iron (II) sulfate heptahydrate, ferrous nitrate, manganese sulfate, zinc sulfate, cobalt sulfate, and calcium carbonate.

In addition, for the inorganic salt, one kind of material can solely be used, or two or more kinds of materials can be combined. The concentration of the inorganic salt in the culture medium (Y) or the culture medium (X) may be suitably adjusted depending on conditions such as the type of the recombinant microorganism used therein, the type and properties of a desired target substance, reaction conditions, and the type of inorganic salt, and therefore, the concentration is not particularly limited, but may be, for example, about 0.01% to 1% w/v.

In addition, as necessary, vitamins can be added to the culture medium (Y) or the culture medium (X). Examples of vitamins include biotin, thiamine (vitamin B1), pyridoxine (vitamin B6), pantothenic acid, and inositol. In addition, regarding the concentration of vitamins in the culture medium (Y) or the culture medium (X), generally, when the culture medium or the reaction medium contains trace amounts of vitamins, favorable growth or metabolic reactions of the microorganism can be secured. Also, when a culture medium base material derived from a natural product such as tryptone, peptone, or soytone is used, there would be may cases where it is not necessarily required to add vitamins artificially to the culture medium (Y) or the culture medium (X) since vitamins are inherently present in such a base material, and thus, in such cases, the concentration of vitamins in the culture medium (Y) or the culture medium (X) would be determined automatically. Therefore, although there is little significance in actively defining the concentration of vitamins in the culture medium (Y) or the culture medium (X), for example, about 0.01 mg to 1 mg of vitamins may be added to 1 L of the culture medium (Y) or the culture medium (X) in order to promote enzyme reactions in the L-leucine biosynthesis pathway.

Meanwhile, although the pH of the culture medium (Y) or the culture medium (X) is not particularly limited as long as it falls within a range in which the reaction for producing a desired target substance proceeds, in general, the pH may be preferably about 6.0 to 8.0, more preferably 6.5 to 8.0, for example, around 7.5.

Additionally, for the culture medium (Y) or the culture medium (X), for example, a natural culture medium for bacterial culture such as an LB culture medium, an NB culture medium, an SCD culture medium, and a TB culture medium may also be used. Alternatively, when the microorganism according to the present invention is a fungal recombinant microorganism, for the culture medium (Y) or the culture medium (X), a potato dextrose agar (PDA) culture medium, a potato sucrose agar (PSA culture medium), an oatmeal culture medium, a malt extract culture medium, a YPD culture medium, an ISP culture medium or the like can be used. In addition, when the recombinant microorganism according to the present invention is a microorganism belonging to a coryneform bacterium, for the culture medium (Y) or the culture medium (X), an A culture medium [WO2020090016], a BT culture medium [Omumasaba, C. A. et al., Corynebacterium glutamicum glyceraldehyde-3-phosphate dehydrogenase isoforms with opposite. ATP-dependent regulation. J. Mol. Microbiol. Biotechnol. 8:91-103 (2004)], an NA culture medium [WO2020208842], a CGXII culture medium described in Examples of this application or the like can be preferably used.

Furthermore, for example, when it is desired to produce a relatively large amount of L-leucine, in order to reduce production costs, various base materials obtained by performing a pretreatment such as a saccharification treatment on various biomasses materials may be used for the culture medium (Y) or the culture medium (X) depending on the type and properties of the recombinant microorganism used therein, and also, those obtained by additionally adding the above ingredients to the various biomasses as needed may be employed.

### [Culture/Incubation Conditions]

The culture/incubation (reaction) temperatures for steps (p′) and (p), respectively, may be within ranges that make it possible to produce L-leucine finally in step (p), and may be appropriately set independently for each of steps (p′) and (p), depending on properties of the recombinant microorganism used or the process product of microbial cells thereof. The temperatures are not particularly limited, but may be typically about 20 to 50° C., preferably about 25 to 47° C., and more preferably about 27 to 37° C. Within such temperature ranges. L-leucine can be efficiently produced. The culture time/incubation time for steps (p′) and (p), respectively, may also be appropriately adjusted so as to realize production of L-leucine, and therefore, are not particularly limited, but may be each independently set to, for example, about 1 hour to about 7 days, and, in consideration of a more efficient target substance, they may be each dependently set to preferably about 1 hour to about 3 days, e.g., about 1 hour to 48 hours, about 12 hours to about 7 days, about 12 hours to about 3 days, or about 12 hours to 48 hours.

The culture/incubation (reaction) in steps (p′) and (p), respectively, may be performed in any of a batch manner, a fed-batch manner, and a continuous manner. Among these, a batch manner is preferable.

In addition, after the reaction in step (p) is completed, the recombinant microorganism, the processed product of microbial cells thereof, etc. may be recovered from the culture medium (X) by a suitable operation such as centrifugal separation, and the recovered recombinant microorganism may be reused to repeat step (p) multiple times. A configuration in which step (p) is repeated multiple times by way of reusing the recombinant microorganism in such a manner is advantageous because the it will lead to reductions in production costs, and realize efficient production of a target substance.

[Step (q)/Other Subsequent Steps]

After a predetermined amount of L-leucine is produced in the culture medium (X) in step (p), a fraction including L-leucine is recovered from the culture medium (X) in step (p).

In this case, for the term “recovering a fraction including L-leucine from the culture medium (X)” in step (p), it may be only required that the term be interpreted literally, and, it may be only required that a fraction that includes an amount of L-leucine suitable for subsequent steps or various purposes be recovered, and therefore, any other conditions such as the form and purity of L-leucine are acceptable. Step (q) may encompass any embodiments for recovering a fraction including L-leucine, e.g., recovering a supernatant (liquid) of the reaction solution, which contains L-leucine, from the mixture obtained after step (p), i.e., the mixture of the culture medium (X) and the microbial cells or the processed product of the microbial cells, based on various solid-liquid separation methods such as decantation, centrifugation, and filtration; or separating the microbial cells or the processed product of the microbial cells containing L-leucine, or crystals of L-leucine obtained through crystallization thereof in the culture medium (X), based on any of various solid-liquid separation methods.

Furthermore, the “fraction including L-leucine” recovered in step (q) may also be subjected to a step of separation and/or purification of L-leucine. For the separation and purification of L-leucine, any suitable separation/purification techniques may be adopted depending on the required purity or the like in consideration of purposes or the of L-leucine. Although the separation/purification techniques are not particularly limited, for example, various crystallization methods, various filtration techniques such as ultrafiltration, various chromatography techniques such as ion exchange chromatography, affinity chromatography, hydrophobic chromatography, and reversed-phase chromatography, concentration methods, dialysis, activated-carbon adsorption methods and the like may be appropriately combined to recover a target substance. Since, for such substance separation/purification techniques, there are various techniques have been known, any known techniques have may be appropriately employed.

Furthermore, the method of the present invention may further include, for example, concentrating, and/or drying, and/or washing, and/or crushing, and/or powdering or granulating, and/or packaging the fraction or a crude product containing L-leucine, in step (q) and/or the subsequent step of separation/production.

Additionally, applications of target substances produced in the present invention are not limited in any ways, but include, for example, pharmaceutical applications, food applications, industrial applications, fuel applications, and cosmetic applications. In addition, the target substances produced in the present invention may be substances actually employed for various applications or may be intermediate raw materials for use in production of final products.

While specific embodiments of the present invention have been described above in detail, the present invention is not limited to the above-described embodiments. Various modifications, alternations, and combinations may be adopted with respect to configurations, elements, and features, without departing from the spirit and scope of the present invention. In addition, all or parts of the embodiments shown for the invention relating to the method, and all or parts of the embodiments shown for the invention relating to the recombinant microorganism (recombinant coryneform bacterium) may be mutually diverted, or mutually substituted, or combined in the invention relating to the method or the recombinant microorganism (recombinant coryneform bacterium) without causing any inconsistencies, and various embodiments resulting from such diversion, substitution or combination are set forth herein. In addition, needless to say, the specific aspects or embodiments for the recombinant microorganism, which are shown for the invention relating to the method, may be adopted for specific aspects or embodiments for the invention relating to the recombinant microorganism, and are set forth herein.

In the meantime, unless otherwise specified, the terms “contains,” “includes.” and “have” used in this application do not exclude the presence of elements other than elements that these terms refer to as objects, and these terms may be used interchangeably.

### EXAMPLES

### (1) Preparation of Expression Plasmid Vector

A Ptac fragment, and an rrnBter fragment were each obtained using the primer pair of GEP470 and GEP476 and the primer pair of GEP467 and GEP468 shown in Table 7, respectively, and using a plasmid vector pFLAG-CTC (sigma) serving as a template based on a PCR method. Next, a plasmid vector named pGEK003 (WO2019156152A) possessed by the applicant was cleaved with BamHI and EcoRV to obtain a linear DNA fragment of pGEK003. The above-mentioned Ptac fragment, rmBter fragment, and the linear DNA fragment were connected to each other in a circular form, using In-Fusion HD Cloning Kit (commercially available from Clontech Laboratories, Inc.), thereby obtaining a plasmid vector named pGE407. Meanwhile, in pGE407, the Ptac-rmBter, the KanR/pUCori, and the pCGlori were arranged in that order.

TABLE 7

| Primer | | SEQ ID |
|---|---|---|
| name | Nucleotide sequence | No |
| GEP470 | 5′-CTGCTCCCAAGGATCGCATCATAACGGTTCTGGCA-3′ | 41 |
| GEP476 | 5′-CCGTCGACGATATCATATGATATCTCCTGTGTGAAA-3′ | 42 |
| GEP467 | 5′- | 43 |
| | TGATATCGTCGACGGATCCTGCCTGGCGGCAGTAGCGCG-3′ | |
| GEP468 | 5′-GAGGCAGACCTCGATAAAAAGGCCATCCGTCAGGA-3′ | 44 |

Based on the same method as described in WO2019156152A, a plasmid vector in which mutations were introduced into the pUCori sequence was obtained using pGEK003 above, serving as a template, and the primer pair of GEP3304 and GEP3305 shown in Table 8, and the obtained plasmid vector was named pGE1722. Furthermore, DNA fragments that had been obtained by cutting pGE1722 and pGEK094 (possessed by the applicant and described in WO2019156152A), respectively, with restriction enzymes Xmal and NotI were mixed, and then, these DNA fragments were ligated using T4 DNA ligase (NEB). A plasmid obtained in this way was named pGE1870. In pGE1870, the Ptac-lacZ-rrnBter, the KanR, the pBR322ori, and the pCGlori were arranged in that order.

TABLE 8

| | | SEQ |
|---|---|---|
| Primer | | ID |
| name | Nucleotide sequence | No |
| GEP3304 | 5′-CTAGAAGGACAGTATTTGGTATCTGC-3′ | 45 |
| GEP3305 | 5′-ATACTGTCCTTCTAGTGTAGCCGTAG-3′ | 46 |

(2) Construction of ilvE Expression Plasmid

Genomic DNA of Corynebacterium glutamicum ATCC13032 (NBRC 12168) was prepared, and then, a region including an ORF of the ilvE gene was amplified from the genomic DNA serving as a template, using the primer pair of GEP4481 and GEP4482 shown in Table 9, based on a PCR method. The DNA fragment amplified in this way and, a DNA fragment obtained by cutting the expression vector pGE1870 with restriction enzymes Ndel and BamHI were mixed, and then, these DNA fragments were connected to each other using In-Fusion HD Cloning Kit. A plasmid obtained in this way was named pGE2677.

In the meantime, in the sequence listing, the nucleotide sequence of the ORF region of the ilvE gene is shown as SEQ ID No. 33, and the amino acid sequence of the branched-chain amino acid aminotransferase encoded by the ilvE gene is shown as SEQ ID No. 34.

TABLE 9

| Primer | | SEQ |
|---|---|---|
| name | Nucleotide sequence | ID No |
| GEP4481 | 5′-ACAGGAGATATCATATGACGTCATTAGAGTTCAC-3′ | 47 |
| GEP4482 | 5′- | 48 |
| | CCGCCAGGCAGGATCCAGATCTTAGCCAACCAGTGGGTAAA-3′ | |

### (3) Construction of Expression Plasmids for Various Amino Acid Dehydrogenase

Next, an amino acid sequence of a phenylalanine dehydrogenase derived from Lysinibacillus sphaericus (UniprotKB: P23307) was obtained, and then, DNA consisting of a nucleotide sequence for the phenylalanine dehydrogenase, having at the 5′ and 3′ ends additional sequences recognized by restriction enzymes Ndel and BamHI, respectively, was synthesized through artificial gene synthesis. A DNA fragment obtained by cutting the above DNA with Ndel and BamHI was mixed with a fragment that had been obtained by way of cutting the expression vector pGE1870 with the same restriction enzymes to remove the lacZ gene therefrom. and then, these fragments were connected to each other using T4 DNA ligase (NEB). The resulting plasmid was named pGE2248.

In the meantime, in the sequence listing, the nucleotide sequence of the ORF region encoding the phenylalanine dehydrogenase derived from Lysinibacillus sphaericus (UniprotKB: P23307) is shown as SEQ ID No. 1, and the amino acid sequence of the phenylalanine dehydrogenase is shown as SEQ ID No. 2.

Introduction of various amino acid substitutions shown in Table 9 was performed upon pGE2248 above, serving as a template, using various predefined primer pairs (Tables 10 and 11), based on the same method as described in WO2019156152A1, thereby obtaining expression plasmids for various mutant type phenylalanine dehydrogenases.

TABLE 10

| Amino acid substitution | Primer pair | Name of produced plasmid |
|---|---|---|
| V310A | GEP3953/GEP3954 | pGE2265 |
| V310C | GEP4249/GEP4250 | pGE2679 |
| G124S | GEP3953/GEP3954 | pGE2678 |
| V310A | GEP4251/GEP4252 | |
| G124S | GEP4247/GEP4248 | pGE2680 |
| V310G | GEP4251/GEP4252 | |

TABLE 11

| | | SEQ |
|---|---|---|
| Primer | | ID |
| name | Nucleotide sequence | No |
| GEP3953 | 5′-ATTCAGGCAGCAGACGAGCTTTACGGT-3′ | 49 |
| GEP3954 | 5′-GTCTGCTGCCTGAATGAGCCCTCCAG-3′ | 50 |
| GEP4249 | 5′-ATTCAGTGCGCAGACGAGCTTTACGGT-3′ | 51 |
| GEP4250 | 5′-GTCTGCGCACTGAATGAGCCCTCCAG-3′ | 52 |
| GEP4251 | 5′-TATACCTCCACTGACATGGGAACTAC-3′ | 53 |
| GEP4252 | 5′-GTCAGTGGAGGTATAGAAGCGCCCAT-3′ | 54 |
| GEP4247 | 5′-ATTCAGGGCGCAGACGAGCTTTACGGT-3′ | 55 |
| GEP4248 | 5′-GTCTGCGCCCTGAATGAGCCCTCCAG-3′ | 56 |

Next, genomic DNA of Sporosarcina ureae (NBRC12699) was prepared, and then, upon this genomic DNA serving as a template, a DNA fragment including a region of the amino acid dehydrogenase gene was amplified using the primer pair of GEP3794 and GEP3795 shown in Table 12 based on a PCR method. Next, a DNA fragment obtained by cutting the above DNA with Ndel and BamHI, and a fragment obtained by way of cutting the expression vector pGE1870 with the same restriction enzymes to remove the lacZ gene therefrom were mixed, and these DNA fragments were connected to each other using In-Fusion HD Cloning Kit, thus obtaining pGE2219, which was an expression plasmid for the amino acid dehydrogenase derived from S. ureae.

In the meantime, in the sequence listing, the nucleotide sequence of the coding region of the amino acid dehydrogenase gene derived from S. ureae is shown as SEQ ID No. 9, and the amino acid sequence of the amino acid dehydrogenase is shown as SEQ ID No. 10.

TABLE 12

| Primer | | SEQ ID |
|---|---|---|
| name | Nucleotide sequence | No |
| GEP3794 | 5′-ACAGGAGATATCATATGATTTTGGTAACTTTAGA-3′ | 57 |
| GEP3795 | 5′-CCGCCAGGCAGGATCCTTACTGATGAAAATTCCATT-3′ | 58 |

Next, genomic DNA of Rhodococcus jostii (NBRC16295) was prepared, and, upon this DNA serving as a template, a DNA fragment including a region of the Glu/Leu/Phe/Val dehydrogenase gene was amplified using the primer pair of GEP3800 and GEP3801 shown in Table 13 based on a PCR method. Next, a DNA fragment obtained by cutting the above DNA with Ndel and BamHI was mixed with a fragment obtained by way of cutting the expression vector pGE1870 with the same restriction enzymes to remove the lacZ gene therefrom, and then, these DNA fragments were connected to each other using In-Fusion HD Cloning Kit, thus obtaining an expression plasmid pGE2216 for the Glu/Leu/Phe/Val dehydrogenase derived from R. jostii.

In the meantime, in the sequence listing, the nucleotide sequence of the coding region of the Glu/Leu/Phe/Val dehydrogenase gene derived from R. jostii is shown as SEQ ID No. 17, and the amino acid sequence of the Glu/Leu/Phe/Val dehydrogenase is shown as SEQ ID No. 18.

TABLE 13

| Primer | | SEQ ID |
|---|---|---|
| name | Nucleotide sequence | No |
| GEP3800 | 5′-ACAGGAGATATCATATGAGACTCGACAAGACATT-3′ | 59 |
| GEP3801 | 5′-CCGCCAGGCAGGATCCTATACGCGGAGCGGAGTCG-3′ | 60 |

Next, an amino acid sequence of the Glu/Leu/Phe/Val dehydrogenase (UniprotKB: Q76GS2, leucine dehydrogenase) derived from Lysinibacillus sphaericus was obtained, and then, DNA consisting of a nucleotide sequence for the Glu/Leu/Phe/Val dehydrogenase, having at the 5′ and 3′ ends additional sequences recognized by restriction enzymes NdeI and BamHI, respectively, was synthesized through artificial gene synthesis. A DNA fragment obtained by cutting this DNA with NdeI and BamHI was mixed with a fragment obtained by way of cutting the expression vector pGE1870 with the same restriction enzymes to remove the lacZ gene therefrom, and then, these fragments were ligated to each other using T4DNA ligase (NEB). The resulting plasmid was named pGE2220.

In the meantime, in the sequence listing, the nucleotide sequence of the ORF region of the Glu/Leu/Phe/Val dehydrogenase gene derived from L. sphaericus is shown was SEQ ID No. 25, and the amino acid sequence of the Glu/Leu/Phe/Val dehydrogenase is shown as SEQ ID No. 26.

Next, sequential introduction of amino acid substitutions of L40K, V291L/V294S and A113G was performed upon pGE2220 above, serving as a starting material of template, using the primer pairs shown in Table 14, based on the same method as described in WO2019156152A1. thereby obtaining an expression plasmid for a mutant of the Glu/Leu/Phe/Val dehydrogenase derived from L. shaericus, and this was named pGE2253.

TABLE 14

| Amino acid | Primer | | SEQ |
|---|---|---|---|
| substitution | name | Nucleotide sequence | ID No |
| L40K | GEP3914 | 5′-CCAGCTAAGGGAGGTGCACGCATGTGG-3′ | 61 |
| | GEP3915 | 5′-ACCTCCCTTAGCTGGTCCCAGAGTAGT-3′ | 62 |
| V291L/ | GEP3922 | 5′- | 63 |
| V294S | | GGCCTGATCAACTCCGCCGACGAGCTTTACGGC-3′ | |
| | GEP3923 | 5′- | 64 |
| | | GGCGGAGTTGATCAGGCCACCAGCGTTGATGAC-3′ | |
| A113G | GEP3916 | 5′-ATCACCGGCGAAGATGTGGGCACAAC-3′ | 65 |
| | GEP3917 | 5′-ATCTTCGCCGGTGATGTATCGGCCGT-3′ | 66 |

### (4) Preparation of Plasmid Vector for Genome Insertion

Upon the above-described genomic DNA of Corynebacterium glutamicum ATCC13032 (NBRC12168) serving as a template, regions upstream and downstream of tnpla, which corresponds to a transposase, in the genome were amplified using the primer pair of GEP694 and GEP695 and the primer pair of GEP696 and GEP697, respectively, shown in Table 15 based on a PCR method. The DNA fragments corresponding to the regions upstream and downstream of tnpla were mixed with pGE015 (JP2020182449A) that had been cleaved using the restriction enzyme EcoRI, and then, these were connected to each other in a circular form using In-Fusion HD Cloning Kit to thus obtain a plasmid vector pGE192.

TABLE 15

| Primer | | SEQ ID |
|---|---|---|
| name | Nucleotide sequence | No |
| GEP694 | 5′-GACGGCCAGTGAATTCTCATTCCAGCGTCACGACG-3′ | 67 |
| GEP695 | 5′-CTCGAGGGGTAGAGCCTTTTGTTGGT-3′ | 68 |
| GEP696 | 5′-GCTCTACCCCTCGAGAACAGGAAGAGCCCGTAAAC-3′ | 69 |
| GEP697 | 5′-TACCGAGCTCGAATTGGGAAGACTCGCCTCTGCGG-3′ | 70 |

Next, an amino acid sequence of an isopropylmalate synthase (IPMS) gene of coryneform bacteria (UniprotKB: P42455) was obtained, and thus, a DNA fragment consisting of a nucleotide sequence, having at the 5′ and 3′ ends additional sequences recognized by restriction enzymes Ndel and BamHI, respectively, was synthesized through artificial gene synthesis. A DNA fragment obtained by digesting the above-obtained DNA fragment with Ndel and BamHI, and a DNA fragment obtained by digesting the expression vector pGE409 (WO2020090016A) with the same restriction enzymes were mixed, and then, these DNA fragments were ligated using T4 DNA ligase (NEB). The resulting plasmid was named pGE1835.

Furthermore, upon pGE1835, introduction of amino acid substitutions of G530D, G532D and A535T was performed in the IPMS coding region using the primer pair of GEP3457 and GEP3458 shown in Table 16, based on the same method as described in WO2019156152A. thereby obtaining a plasmid vector pGE1881 encoding a mutant type IPMS.

Next, a DNA fragment was amplified from pGE1881 serving as a template, using the primer pair of GEP711 and GEP3624 shown in Table 16, based on a PCR method. The resulting DNA fragment, and linear DNA of pGE192 that had been digested with the restriction enzyme Xhol were connected to each other in a circular form using In-Fusion HD Cloning Kit (commercially available from Clontech Laboratories, Inc.) to obtain a plasmid vector pGE2003 for genome insertion of a feedback-resistant IPMS gene.

TABLE 16

| Primer | | SEQ ID |
|---|---|---|
| name | Nucleotide sequence | No |
| GEP3457 | 5′-ACAATGACCCTTTGACCGCCTATGCTAACGCAC-3′ | 71 |
| GEP3458 | 5′-TCAAAGGGTCATTGTCACGGCCGTCAACGGTGA-3′ | 72 |
| GEP711 | 5′-GGCTCTACCCCTCGAGAAGAAATTTAGATGATTGA-3′ | 73 |
| GEP3624 | 5′-TCTTCCTGTTCTCGATTCACCGACAAACAACAG-3′ | 74 |

Next, a linear DNA fragment that had been obtained by cutting pGE409 described in WO2020090016A with Ndel and BamHI, and a LacZ gene fragment that had been obtained through cleavage of pGEK020 (WO2019156152A1) with the same restriction enzymes were mixed, and then, these DNA fragments were ligated to each other using T4DNA ligase (NEB). Accordingly, the DNA fragments were circularized to thereby obtain a plasmid vector pGE421 containing PgapA-lacZ-rmnBter, KanR, pUCori, and pCGlori in that order.

Next, a PgapA-lacZ-rmnBter fragment was amplified from pGE421 above, serving as a template, using the primer pair of GEP745 and GEP936 shown in Table 17, based on a PCR method. Next, regions upstream and downstream of tnp2c (transposase) within the genome of Corynebacterium glutamicum strain ATCC13032 were amplified from its genomic DNA, serving as a template, using the primer pair of GEP943 and GEP961 and the primer pair of GEP945 and GEP946, respectively, shown in Table 17, based on a PCR method.

TABLE 17

| Primer | | SEQ |
|---|---|---|
| name | Nucleotide sequence | ID No |
| GEP745 | 5′-GGTACCGAAGAAATTTAGATGATTG-3′ | 75 |
| GEP936 | 5′-ACTAGTAAAAAGGCCATCCGTCAGGA-3′ | 76 |
| GEP943 | 5′-GTGAATTCGAGCTCGTCACACCTTCTGCCACGTCA-3′ | 77 |
| GEP961 | 5′- | 78 |
| | AATTTCTTCGGTACCACCTTGATTGATCATGTCGAGGAAAGCC-3′ | |
| GEP945 | 5′-GGCCTTTTTACTAGTTGAATTCGCCGTCAGTTCCG-3′ | 79 |
| GEP946 | 5′-AGGTGGATACGGATCAGGCTATTGTGTCTATCAGG-3′ | 80 |

The PgapA-lacZ-rrnBter fragment obtained as described above, and fragments of the regions of upstream and downstream of tnp2c (transposase) were mixed with pGE209 (WO2019156152A1) that had been cleaved with the restriction enzymes KpnI and BamHI, and then, these fragments were connected to one another in a circular form using In-Fusion HD Cloning Kit to thereby obtain pGE235, which was a plasmid vector for disruption of the tnp2c gene.

A DNA fragment including a gapA promoter region was amplified from genomic DNA of Corynebacterium glutamicum strain ATCC13032, serving as a template, using the primer pair of GEP475 and GEP478 shown in Table 18, based on a PCR method. In addition, a DNA fragment including a predetermined region of a plasmid vector pFLAG-CTC (sigma) was amplified from the plasmid vector serving as a template, using the primer pair of GEP467 and GEP468 shown in Table 7, based on a PCR method. The above-obtained DNA fragments were mixed with pGE409 (WO2020090016A1) that had been cleaved with BamHI and EcoRV, and then, these were connected to one another in a circular form, using In-Fusion HD Cloning Kit, to thus obtain a plasmid vector pGE412. Next, DNA fragments each including gene coding regions for a mutant type acetohydroxyacid synthase (AHAS) into which feedback-resistant mutations were introduced were amplified from the genomic DNA of Corynebacterium glutamicum strain ATCC13032 serving as a template, using the primer pair of GEP2947 and GEP3208 and the primer pair of GEP3207 and GEP2961, respectively, based on a PCR method. The resulting DNA fragments were mixed with pGE412 that had been cleaved with Ndel, and these were connected to one another in a circle form, using In-Fusion HD Cloning Kit, to thus obtain a plasmid vector pGE1597.

In addition, a DNA fragment including a predetermined region of pGE1597 was amplified from pGE1597 serving as a template, using the primer pair of GEP3226 and GEP3227 shown in Table 18, based on a PCR method. The resulting DNA fragment was connected to a linear DNA fragment obtained by digesting pGE421 above with Ndel and BamHI, in a circular form, using In-Fusion HD Cloning Kit, thus obtaining a plasmid vector pGE1630.

Next, a linear DNA fragment obtained by cutting pGE235 above with Ndel and BamHI, and a linear DNA fragment obtained by cutting pGE1630 above with the same restriction enzymes were ligated to each other using T4 DNA ligase (NEB) to thus obtain a plasmid vector pGE1899 for genome insertion of a feedback-resistant acetohydroxyacid synthase (AHAS) gene.

In addition, the feedback-resistant acetohydroxyacid synthase (AHAS) consisted of a large unit (ilvB) and a small unit (ilvN) and had three amino acid substitutions of 20D/121D/122F in the moiety of the small unit (ilvN). In the meantime, nucleotide sequences and amino acid sequences for the wild type large unit (ilvB) and the wild type small unit (ilvN) of Corynebacterium glutamicum strain ATCC13032 are shown in SEQ ID Nos. 37 and 38, and SEQ ID Nos. 39 and 40, respectively.

TABLE 18

| Primer | | SEQ ID |
|---|---|---|
| name | Nucleotide sequence | No |
| GEP475 | 5′-ACCAAGTCAGGGATCGAAGAAATTTAGATGATTGA-3′ | 81 |
| GEP478 | 5′-CCGCCAGGCAGGATCCAACTACATTATTGTTCTAC-3′ | 82 |
| GEP3226 | 5′-CCGCCAGGCAGGATCCAACTACATTATTGTTCTAC-3′ | 83 |
| GEP3227 | 5′- | 84 |
| | AGAGGAGACACCATATGAACGTGGCAGCTTCTCAAC-3′ | |
| GEP2947 | 5′-AGAGGAGACACCATAATGAATGTGGCAGCTTCTC-3′ | 85 |
| GEP3208 | 5′-GGAAAAGTCATCGTCTACGTCCTGAACGAGTAC-3′ | 86 |
| GEP3207 | 5′-GACGATGACTTTTCCCGCGTATCAGGTATGTTC-3′ | 87 |
| GEP2961 | 5′-GTCGACGATATCATATTAAGCGGTTTCTGCGCG-3′ | 88 |

Regions upstream and downstream of the branched-chain amino acid aminotransferase gene (ilvE) were amplified from the genomic DNA of Corynebacterium glutamicum strain ATCC13032 serving as a template, using the primer pair of GEP3381 and GEP3382 and the primer pair of GEP3383 and GEP3384, respectively, shown in Table 19, based on a PCR method. The respective DNA fragments of the regions upstream and downstream of the above-mentioned gene were mixed with a linear DNA fragment that had been obtained by way of cutting pGE209 (WO2019156152A1) with the restriction enzymes KpnI and BamHI in advance, and these DNA fragments were connected to one another in a circular form, using In-Fusion HD Cloning Kit, to thus obtain pGE1782, which was a plasmid vector for disruption of ilvE.

TABLE 19

| Primer | | SEQ ID |
|---|---|---|
| name | Nucleotide sequence | No |
| GEP3381 | 5′-GTGAATTCGAGCTCGATGCGCAGCAGACGAGG-3′ | 89 |
| GEP3382 | 5′-GATTTAGCTAGCCAATTTCGTCTCACGG-3′ | 90 |
| GEP3383 | 5′-TTGGCTAGCTAAATCAACCGGTTTTAA-3′ | 91 |
| GEP3384 | 5′-AGGTGGATACGGATCGCGAGGTGTAATACGC-3′ | 92 |

### (5) Preparation of Leucine-Producing Strains

First of all, pGE2003 above was introduced into Corynebacterium glutamicum strain ATCC13032 by way of electroporation, and thus, a feedback-resistant IPMS-expressing strain was prepared based on a marker-less gene recombination method described in WO2019156152A, and was named strain GESs613. In the same manner, pGE1899 above was introduced into the strain GESs613, and thus, a feedback-resistant AHAS-expressing strain was prepared, and was named strain GESs637. Furthermore, pGE1782 above was introduced into the strain GESs637, and thus, a ΔilvE strain was prepared, and was named strain GESs655.

Next, the above expression plasmids were each electroporated into the strain GESs655, and thus, various amino acid dehydrogenase-expressing strains were obtained through kanamycin-based selection (Table 20).

### (6) Leucine Production Test

The transformants obtained as described above were each streaked onto A agar culture media (WO2020090016A1) or A/Kan agar culture media (A agar culture media with kanamycin (25 μg/mL)), and then, the transformants were cultured at 18 to 33° C. Each of the respective proliferated microbial cells were inoculated into 10 mL of a CGXII culture medium (glucose 40 g/L, (NH₄)₂SO₄ 20 g/L, urea 5 g/L, KH₂PO₄ 1 g/L, K₂HPO₄ 1 g/L, MgSO₄·7H₂O 0.25 g/L, MOPS 42 g/L, CaCl₂) 10 mg/L, FeSO₄·7H₂O 10 mg/L, MnSO₄·H₂O 10 mg/L, ZnSO₄·7H₂O 1 mg/L. CuSO₄ 0.2 mg/L, NiCl₂·6H₂O 0.02 mg/L, biotin 0.2 mg/L, thiamine hydrochloride 0.2 mg/L, PCA 30 mg/L. pH 7.4) to an OD₆₁₀ of 0.2, and then, these were cultured with shaking at 200 rpm at 33° C. for 48 hours.

After the culture of the transformants was completed, each culture solution was separated into a supernatant and a pellet based on centrifugation, and then, according to the method described in WO2020090016A, the concentrations of various amino acids in the supernatant were analyzed by high performance liquid chromatography. In addition, as necessary, the pellet was suspended in a crushing buffer (50 mM Tris(7.5), 100 mM KCl, 1 mM EDTA). According to the method described in WO2020090016A, microbial cells therein were then crushed with a multi-bead shocker, the concentration of proteins in the supernatant was quantified based on the Bradford method, and also, the resulting specimen was analyzed by SDS-PAGE, to thereby confirm the expression levels of the various enzyme genes introduced into the hosts in protein levels.

### (Results)

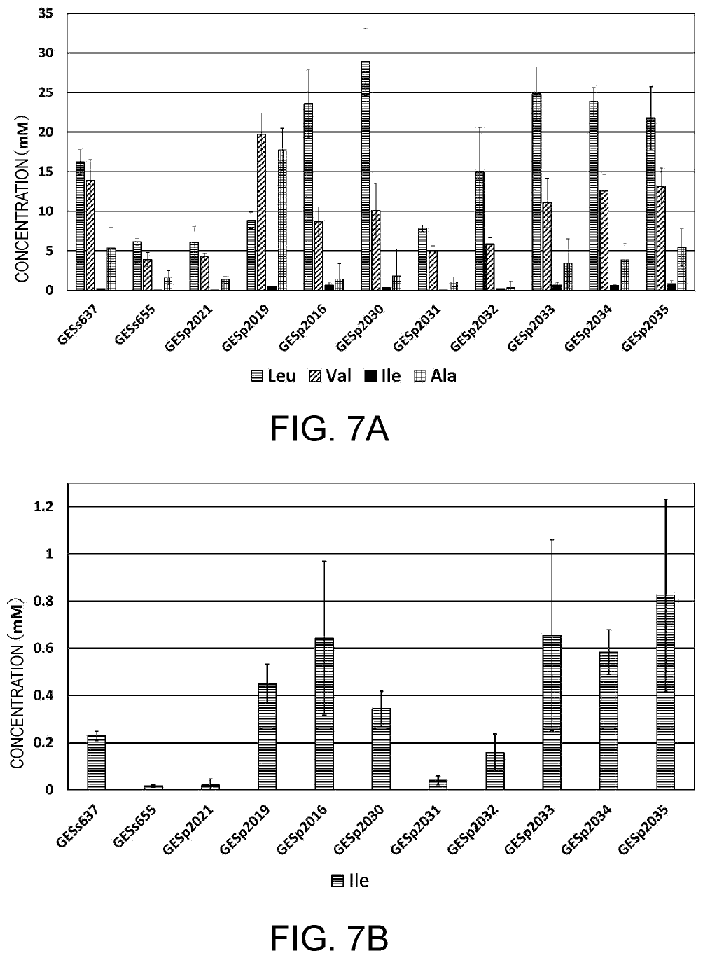

The names of strains and the overview of genes expressed therein according to test examples and comparative test examples (control), and analysis results of various amino acid concentrations in the supernatants for the respective strain samples are shown in Table 20 and FIGS. 7A and 7B.

In addition, based on the Bradford method and SDS-PAGE analysis performed on the above-mentioned supernatant samples of the culture solutions, it was revealed that the various enzyme genes introduced into the hosts were expressed in protein level

TABLE 20
Amino acid production profile of amino acid dehydrogenase-expressing strains

| | Expression | | |
|---|---|---|---|
| Strain name | plasmid | Overview of expressed gene/deficient gene | |
| GESs637 | — | Δtnp2c::PgapA-ilvBN(M13), Δtnp1a::PgapA- | |
| | | Cg_leuA(G530D/G532D/A535T) | |
| | | * Native ilvE remaining | |
| GESs655 | — | Δtnp2c::PgapA-ilvBN(M13), Δtnp1a::PgapA- | |
| | | Cg_leuA(G530D/G532D/A535T) ΔilvE | |
| | | * a strain obtained by knocking out ilvE in GESs637. | |
| GESp2021 | pGE407 | empty vector | Those obtained by |
| GESp2019 | pGE2677 | ilvE | introducing the various |
| GESp2016 | pGE2265 | Ls_pheDH (V310A) | expression plasmids |
| GESp2030 | pGE2216 | Rj_leuDH | (left) into |
| GESp2031 | pGE2219 | Su_pheDH | GESs655(ΔilvE). |
| GESp2032 | pGE2253 | Ls_leuDH | |
| | | (L40K/A113G/V291L/V294S) | |
| GESp2033 | pGE2678 | Ls_pheDH (G124S/V310A) | |
| GESp2034 | pGE2679 | Ls_pheDH (V310C) | |
| GESp2035 | pGE2680 | Ls_pheDH (G124S/V310G) | |

| Strain | Expression | L-leucine*¹ | | L-valine*¹ | |
|---|---|---|---|---|---|
| name | plasmid | (mM) | | (mM) | |
| GESs637 | — | 16 ± 0.79 | 16 ± 1.6 | 13.8 ± 1.3 | 13.8 ± 2.7 |
| GESs655 | — | 6.1 ± 0.28 | 6.1 ± 0.48 | 3.9 ± 0.5 | 3.9 ± 0.90 |
| GESp2021 | pGE407 | 6.0 ± 1.0 | 6.0 ± 2.0 | 4.3 ± 0.23 | 4.3 ± 0.45 |
| GESp2019 | pGE2677 | 8.8 ± 0.37 | 8.8 ± 1.0 | 20 ± 0.95 | 20 ± 2.7 |
| GESp2016 | pGE2265 | 24 ± 1.4 | 24 ± 4.3 | 8.7 ± 0.64 | 8.7 ± 1.9 |
| GESp2030 | pGE2216 | 29 ± 1.6 | 29 ± 4.2 | 10 ± 1.3 | 10 ± 3.4 |
| GESp2031 | pGE2219 | 7.9 ± 0.21 | 7.9 ± 0.42 | 5.0 ± 0.32 | 5.0 ± 0.64 |
| GESp2032 | pGE2253 | 15 ± 2.8 | 15 ± 5.5 | 5.8 ± 0.43 | 5.8 ± 0.85 |
| GESp2033 | pGE2678 | 25 ± 1.7 | 25 ± 3.4 | 11 ± 1.5 | 11 ± 3.1 |
| GESp2034 | pGE2679 | 24 ± 0.88 | 24 ± 1.8 | 13 ± 1.0 | 13 ± 2.1 |
| GESp2035 | pGE2680 | 22 ± 2.3 | 22 ± 4.0 | 13 ± 1.3 | 13 ± 2.3 |

| Strain | L-isoleucine*¹ | | L-alanine*¹ | |
|---|---|---|---|---|
| name | (mM) | | (mM) | |
| GESs637 | 0.23 ± 0.0098 | 0.23 ± 0.020 | 5.3 ± 1.3 | 5.3 ± 2.7 |
| GESs655 | 0.017 ± 0.0033 | 0.017 ± 0.0058 | 1.6 ± 0.52 | 1.6 ± 0.90 |
| GESp2021 | 0.020 ± 0.013*² | 0.020 ± 0.027*² | 1.4 ± 0.23 | 1.4 ± 0.45 |
| GESp2019 | 0.45 ± 0.029 | 0.45 ± 0.082 | 18 ± 0.95 | 18 ± 2.7 |
| GESp2016 | 0.64 ± 0.11 | 0.64 ± 0.33 | 1.5 ± 0.64 | 1.5 ± 1.9 |
| GESp2030 | 0.34 ± 0.028 | 0.34 ± 0.073 | 1.9 ± 1.3 | 1.9 ± 3.4 |
| GESp2031 | 0.040 ± 0.0096 | 0.040 ± 0.019 | 1.1 ± 0.32 | 1.1 ± 0.64 |
| GESp2032 | 0.16 ± 0.040 | 0.16 ± 0.080 | 0.33 ± 0.43 | 0.33 ± 0.85 |
| GESp2033 | 0.65 ± 0.20 | 0.65 ± 0.40 | 3.4 ± 1.5 | 3.4 ± 3.1 |
| GESp2034 | 0.58 ± 0.047 | 0.58 ± 0.095 | 3.8 ± 1.0 | 3.8 ± 2.1 |
| GESp2035 | 0.82 ± 0.23 | 0.82 ± 0.41 | 5.4 ± 1.3 | 5.4 ± 2.3 |

*¹each left column indicates average value ± standard error, and each right column indicates average value ± standard deviation.
*²since peaks of HPLC were small, three values among quantitative values at n = 4 were output as 0 in the software used. Therefore, the confirmed peaks were enlarged, quantitative values were calculated therefrom based on a manual operation, and then, the respective values were calculated from the quantitative values.

As can be understood from the results shown in Table 20 and FIG. 7A and FIG. 7B, it was shown that GESp2016, GESp2030, GESp2032, GESp2033, GESp2034 and GESp2035, in which the various amino acid dehydrogenases were each forcibly expressed, exhibited increased productions of L-leucine, and thus, were capable of realizing efficient production of L-leucine, compared to GESs637, GESs655, GESp2021 and GESp2019, in which any amino acid dehydrogenases were not forcibly expressed. In particular, it was confirmed that GEPp2016, GEPp2030, GESp2033, GESp2034, and GESp2035 exhibited significant increases in productions of leucine, compared to the controls.

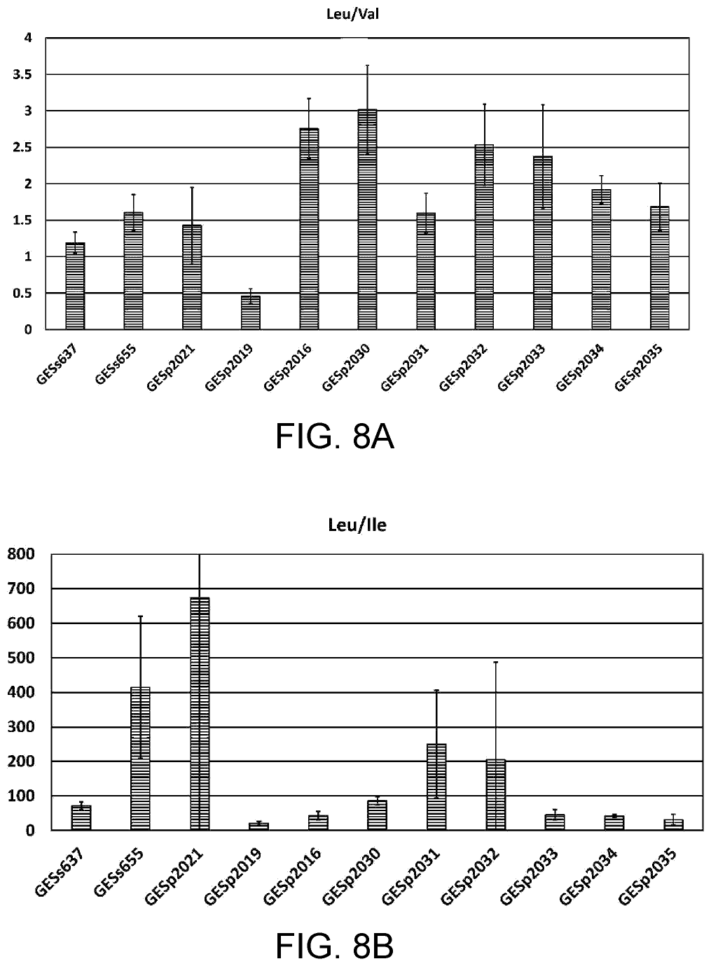

Furthermore, since the secondary production of L-valine from 3-methyl-2-oxobutanoic acid, which is an intermediate metabolite, ((ix) in FIG. 1) was a problem in the L-leucine biosynthesis pathway, ratios of the L-leucine concentration/L-valine concentration (average values of data with standard deviation, hereinafter referred to as an “Leu/Val ratio”) were each calculated for the samples in order to evaluate production efficiencies of L-leucine relative to the secondary production of L-valine. The results are shown in Table 21 and FIG. 8A. In addition, since the secondary production of L-isoleucine from(S)-3-methyl-2-oxopentanoic acid, which is an intermediate metabolite, ((xi) in FIG. 1) was also a problem in the L-leucine biosynthesis pathway, ratios of the L-leucine concentration/L-isoleucine concentration (average value of data with standard deviation, hereinafter referred to as an “Leu/Ile ratio”) were also each calculated for the samples in order to evaluate production efficiencies of L-leucine relative to the secondary production of L-isoleucine. The results are shown in Table 21 and FIG. 8B.

TABLE 21

| Strain | L-leucine/ | | L-leucine/ | |
|---|---|---|---|---|
| name | L-valine* | | L-isoleucine* | |
| GESs637 | 1.2 ± 0.073 | 1.2 ± 0.15 | 72 ± 5.7 | 72 ± 11 |
| GESs655 | 1.6 ± 0.14 | 1.6 ± 0.25 | 410 ± 120 | 410 ± 210 |
| GESp2021 | 1.4 ± 0.26 | 1.4 ± 0.53 | 670 ± 370 | 670 ± 730 |
| GESp2019 | 0.46 ± 0.036 | 0.46 ± 0.10 | 20 ± 1.9 | 20 ± 5.5 |
| GESp2016 | 2.8 ± 0.14 | 2.8 ± 0.41 | 42 ± 4.2 | 42 ± 13 |
| GESp2030 | 3.0 ± 0.23 | 3.0 ± 0.61 | 86 ± 11 | 86 ± 11 |
| GESp2031 | 1.6 ± 0.14 | 1.6 ± 0.14 | 250 ± 78 | 250 ± 160 |
| GESp2032 | 2.5 ± 0.28 | 2.5 ± 0.0.55 | 200 ± 140 | 200 ± 280 |
| GESp2033 | 2.4 ± 0.36 | 2.4 ± 0.71 | 45 ± 7.8 | 45 ± 16 |
| GESp2034 | 1.9 ± 0.096 | 1.9 ± 0.19 | 41 ± 2.3 | 41 ± 4.6 |
| GESp2035 | 1.7 ± 0.19 | 1.7 ± 0.33 | 31 ± 8.7 | 31 ± 15 |

*each left column indicates average value ± standard error, and each right column indicates average value ± standard deviation.

As shown in Table 21 and FIG. 8A, it can be found that GESp2016, GESp2030, GESp2031, GESp2032, GESp2033, GESp2034 and GESp2035, in which the ilvE on the genome was disrupted and into which the various aspartate dehydrogenase genes were each introduced in place of the ilvE in expressible manners, exhibited larger values of the Leu/Val ratios compared to values of the ratios of control strains. That is, it can be understood that, in these strains, the secondary productions of valine were effectively reduced relative to the productions of L-leucine, and thus, it was revealed that, according to aspects of the recombinant strains, L-leucine could be efficiently produced while the secondary productions of valine could be effectively reduced. Furthermore, GESp2016, GESp2030, GESp2032, and GESp2033 each had significantly high values of the Leu/Val ratios, and thus, according to aspects of these strains, L-leucine could be efficiently produced while the secondary productions of valine could be significantly reduced. Additionally, it was recognized that, among these strains, GESp2016, GESp2030, and GESp2032 each exhibited significantly high values of the Leu/Val ratios, and also, significant increases in the productions of leucine. Thus these strains were capable of efficiently producing L-leucine with high yields while significantly reducing secondary productions of valine.

In addition, GESp2030, GESp2031, and GESp2032 exhibited relatively high values of the Leu/Ile ratios as shown in Table 21 and FIG. 8B, and thus, it was recognized that GESp2030, GESp2031, and GESp2032 also had specific effects of effectively reducing secondary productions of isoleucine, besides effectively secondary productions of valine. In particular, it was confirmed that the effects of reducing secondary productions of leucine in GESp2031 and GESp2032 were significant.

As described above, it was shown that, when genes for various aspartate dehydrogenase were introduced into microorganisms having an L-leucine biosynthesis pathway, in expressible manners, efficient production of L-leucine became possible, and that, in specific embodiments, L-leucine could be efficiently produced while secondary productions of valine could be effectively reduced.

### (Supplement)

In examples, as described above, there were steps using In-Fusion cloning kit (commercially available from Takara Bio Inc.) for cloning the coding regions for the various genes, the promoter regions, and the like, and, with regard to the primer pairs used for the PCR amplification in the steps, provided is a supplemental explanation that appropriate adapter sequences were provided at the 5′ ends of the forward/reverse primers in accordance with the instructions of the cloning kit.

### INDUSTRIAL APPLICABILITY

The present invention has high industrial applicability in the field of biotechnology, the field of substance production and the like.

1.-29. (canceled)

30. A method for producing L-leucine, comprising:
(p) incubating a recombinant L-leucine-producing microorganism or a processed product of microbial cells thereof in a predetermined culture medium (X) to thereby produce L-leucine; and
(q) recovering a fraction including the L-leucine from a culture obtained in step (p),
wherein the recombinant microorganism comprises in an expressible form a gene encoding an amino acid dehydrogenase, and the amino acid dehydrogenase has at least catalytic activity on an L-leucine production reaction.

31. The method according to claim 30, wherein the L-leucine production reaction is a reaction represented by the following reaction formula (IX-1) or (IX-2):
$$4 - methyl - 2 - oxopentanoic acid + {NH}_{3} + NADH + {H}^{+} ⇔ L - leucine + {H}_{2} O + {NAD}^{+} \qquad ( IX - 1 )$$
$$4 - methyl - 2 - oxopentanoic acid + {NH}_{3} + NADH + {H}^{+} ⇔ L - leucine + {H}_{2} O + {NADP}^{+} . \qquad ( IX - 2 )$$

32. The method according to claim 31, wherein the L-leucine production reaction is the reaction represented by the reaction formula (IX-1).

33. The method according to claim 30, wherein the recombinant microorganism has a metabolic pathway capable of synthesizing L-leucine via 3-methyl-2-oxobutanoic acid and 4-methyl-2-oxopentanoic acid serving as intermediate metabolites, and the amino acid dehydrogenase has catalytic activity on the reaction that produces L-leucine from 4-methyl-2-oxopentanoic acid.

34. The method according to claim 30, wherein the amino acid dehydrogenase is a bacterial amino acid dehydrogenase.

35. The method according to claim 30, wherein the amino acid dehydrogenase is a wild type amino acid dehydrogenase derived from Lysinibacillus sphaericus, Sporosarcina ureae or Rhodococcus jostii or a mutant type amino acid dehydrogenase thereof.

36. The method according to claim 30, wherein the gene encoding the amino acid dehydrogenase is an exogenous or heterologous gene encoding a bacterial amino acid dehydrogenase having enzyme activity defined in EC1.4.1.9.

37. The method according to claim 36, wherein the bacterial amino acid dehydrogenase is a mutant type amino acid dehydrogenase that consists of an amino acid sequence having at least one of amino acid substitutions of (a), (b) and (c) below based on the amino acid sequence shown in SEQ ID No. 2, or having at least one of amino acid substitutions of (d), (e), (f) and (g) below based on the amino acid sequence shown in SEQ ID No. 26:
(a) an amino acid substitution of an amino acid corresponding to the 51 st leucine (L) with lysine (K),
(b) an amino acid substitution of an amino acid corresponding to the 124th glycine (G) with serine(S),
(c) an amino acid substitution of an amino acid corresponding to the 310th valine (V) with alanine (A), serine(S) or glycine (G),
(d) an amino acid substitution of an amino acid corresponding to the 40th leucine (L) with lysine (K),
(e) an amino acid substitution of an amino acid corresponding to the 113th alanine (A) with glycine (G),
(f) an amino acid substitution of an amino acid corresponding to the 291st valine (V) with leucine (L),
(g) an amino acid substitution of an amino acid corresponding to the 294th valine (V) with alanine (A), serine(S) or glycine (G),
with the proviso that, in each of (a) to (g) above, the amino acid before substitution and the amino acid after substitution, when present, are different from each other.

38. The method according to claim 36, wherein the bacterial amino acid dehydrogenase consists of an amino acid sequence shown in any one of the following (A) to (F):
(A) an amino acid sequence shown in any one of SEQ ID Nos. 2 to 8, 10, 12 to 16, 18 to 22, 24, 26 to 30 and 32;
(B) an amino acid sequence having at least one of amino acid substitutions of (a), (b) and (c) below based on the amino acid sequence shown in SEQ ID No. 2, or having at least one of amino acid substitutions of (d), (e), (f) and (g) below based on the amino acid sequence shown in SEQ ID No. 26, relative to the amino acid sequence shown in any one of SEQ ID Nos. 2 to 8, 10, 12 to 16, 18 to 22, 24, 26 to 30 and 32,
(a) an amino acid substitution of an amino acid corresponding to the 51 st leucine (L) with lysine (K),
(b) an amino acid substitution of an amino acid corresponding to the 124th glycine (G) with serine(S),
(c) an amino acid substitution of an amino acid corresponding to the 310th valine (V) with alanine (A), serine(S) or glycine (G),
(d) an amino acid substitution of an amino acid corresponding to the 40th leucine (L) with lysine (K),
(e) an amino acid substitution of an amino acid corresponding to the 113th alanine (A) with glycine (G),
(f) an amino acid substitution of an amino acid corresponding to the 291st valine (V) with leucine (L),
(g) an amino acid substitution of an amino acid corresponding to the 294th valine (V) with alanine (A), serine(S) or glycine (G),
with the proviso that, in each of (a) to (g) above, the amino acid before substitution and the amino acid after substitution, when present, are different from each other;
(C) an amino acid sequence having a deletion, substitution and/or addition of one or more amino acids relative to the amino acid sequence set forth in (A) or (B) above; and
(D) an amino acid sequence having at least 60% sequence identity to the amino acid sequence set forth in (A) or (B) above,
with the proviso that, in each of the amino acid sequences set forth in (C) and (D) above, the amino acid substitution(s) set forth in (B) above, when present, are maintained.

39. The method according to claim 36, wherein the bacterial amino acid dehydrogenase consists of an amino acid sequence shown in any one of the following (A) to (F):
(A) an amino acid sequence shown in any one of SEQ ID Nos. 2, 10, 18 or 26;
(B) an amino acid sequence having at least one of amino acid substitutions of (a), (b) and (c) below based on the amino acid sequence shown in SEQ ID No. 2, or having at least one of amino acid substitutions of (d), (e), (f) and (g) below based on the amino acid sequence shown in SEQ ID No. 26, relative to the amino acid sequence shown in any one of SEQ ID Nos. 2, 10, 18 or 26,
(a) an amino acid substitution of an amino acid corresponding to the 51st leucine (L) with lysine (K),
(b) an amino acid substitution of an amino acid corresponding to the 124th glycine (G) with serine(S),
(c) an amino acid substitution of an amino acid corresponding to the 310th valine (V) with alanine (A), serine(S) or glycine (G),
(d) an amino acid substitution of an amino acid corresponding to the 40th leucine (L) with lysine (K),
(e) an amino acid substitution of an amino acid corresponding to the 113th alanine (A) with glycine (G),
(f) an amino acid substitution of an amino acid corresponding to the 291 st valine (V) with leucine (L),
(g) an amino acid substitution of an amino acid corresponding to the 294th valine (V) with alanine (A), serine(S) or glycine (G),
with the proviso that, in each of (a) to (g) above, the amino acid before substitution and the amino acid after substitution, when present, are different from each other;
(C) an amino acid sequence having a deletion, substitution and/or addition of one or more amino acids relative to the amino acid sequence set forth in (A) or (B) above; and
(D) an amino acid sequence having at least 60% sequence identity to the amino acid sequence set forth in (A) or (B) above,
with the proviso that, in each of the amino acid sequences set forth in (C) and (D) above, the amino acid substitution(s) set forth in (B) above, when present, are maintained.

40. The method according to claim 36, wherein the bacterial amino acid dehydrogenase consists of an amino acid sequence shown in any one of the following (G1) to (I):
(G1) an amino acid sequence shown in SEQ ID No. 10 or 18;
(G2) an amino acid sequence having an amino acid substitution of V310A or V310C relative to the amino acid sequence shown in SEQ ID No. 2;
(G3) an amino acid sequence having a combination of amino acid substitutions of G124S and V310A or V310G relative to the amino acid sequence shown in SEQ ID No. 2;
(G4) an amino acid sequence having a combination of amino acid substitutions of L40K, A113G, V291L, and V294S relative to the amino acid sequence shown in SEQ ID No. 26;
(H) an amino acid sequence having a deletion, substitution and/or addition of one or more amino acids relative to the amino acid sequence set forth in any one of (G1) to (G4) above; and
(I) an amino acid sequence having at least 60% sequence identity to the amino acid sequence set forth in any one of (G1) to (G4) above,
with the proviso that, in each of (H) and (I) above, the amino acid substitution(s) set forth in each of (G2) to (G4) above, when present, are maintained.

41. The method according to claim 40, wherein the amino acid sequence set forth in (H) above has a deletion, substitution and/or addition of 1 to 50 acids relative to the amino acid sequence set forth in any one of (G1) to (G4) above, and the amino acid sequence set forth in (I) above has at least 85% sequence identity to the amino acid sequence set forth in any one of (G1) to (G4) above.

42. The method according to claim 41, wherein the recombinant microorganism is a coryneform bacterium that comprises in a forcibly expressible manner:
(i) a gene encoding a mutant type acetolactate synthase that has resistance to feedback inhibition of enzyme activity by at least one of valine, leucine and isoleucine;
(iv) a gene encoding a mutant type 2-isopropylmalate synthase that has resistance to feedback inhibition of enzyme activity by L-leucine; and
(ix) the gene encoding the amino acid dehydrogenase,
wherein an ilvE gene or an ortholog thereof on present on the genome has been inactivated or deficient in the recombinant microorganism.

43. The method according to claim 42, wherein the amino acid sequence set forth in (G1) above is the amino acid sequence shown in SEQ ID No. 18.

44. The method according to claim 30, wherein the recombinant microorganism is a bacterium.

45. The method according to claim 30, wherein the recombinant microorganism is a coryneform bacterium.

46. The method according to claim 30, wherein the recombinant microorganism has reduced or inactivated activity of at least one of enzymes defined in EC1.4.1.8, EC1.4.1.23, EC2.6.1.42 and EC2.6.1.66, respectively, wherein the enzymes catalyze a reaction secondarily producing L-valine from 3-methyl-2-oxobutanoic acid.

47. The method according to claim 30, wherein the recombinant microorganism has reduced or inactivated activity of an enzyme defined in EC2.6.1.42, wherein the enzyme catalyzes a reaction secondarily producing L-valine from 3-methyl-2-oxobutanoic acid.

48. The method according to claim 30, wherein the recombinant microorganism exhibits a production ratio of L-leucine/L-valine increased compared to a control microorganism.

49. The method according to claim 48, wherein the recombinant microorganism is a coryneform bacterium that comprises in a forcibly expressible manner:
(i) a gene encoding a mutant type acetolactate synthase that has resistance to feedback inhibition of enzyme activity by at least one of valine, leucine and isoleucine;
(iv) a gene encoding a mutant type 2-isopropylmalate synthase that has resistance to feedback inhibition of enzyme activity by L-leucine; and
(ix) the gene encoding the amino acid dehydrogenase,
wherein an ilvE gene or an ortholog thereof on present on the genome has been inactivated or deficient in the recombinant microorganism.

50. The method according to claim 49, wherein the recombinant microorganism comprises in a forcibly expressible manner a gene encoding a protein defined in (J1) or (J2) below and a gene encoding a protein defined in (K3) or (K4) below serving as the gene set forth in (i) above, and a gene encoding a protein defined in (L3) or (L4) below serving as the gene set forth in (iv) above:
(J1) a protein consisting of an amino acid sequence shown in SEQ ID No. 38;
(J2) a protein consisting of an amino acid sequence having a deletion, substitution and/or addition of one or more amino acids relative to the amino acid sequence shown in SEQ ID No. 38, the protein being capable of functioning as an acetolactate synthase large subunit;
(K3) a protein consisting of an amino acid sequence having amino acid substitutions of G20D, 121D and I22F relative to the amino acid sequence shown in SEQ ID No. 40;
(K4) a protein consisting of an amino acid sequence having a deletion, substitution and/or addition of one or more amino acids relative to the amino acid sequence of the protein set forth in (K3) above, the protein functioning as an acetolactate synthase small subunit and causing the resulting acetolactate synthase to exhibit resistance to feedback inhibition of enzyme activity by at least one of valine, leucine and isoleucine, with the proviso that the amino acid substitutions of G20D, 121D and I22F are maintained;
(L3) a protein consisting of an amino acid sequence having amino acid substitutions of G530D, G532D and A535T relative to the amino acid sequence shown in SEQ ID No. 36; and
(L4) a protein consisting of an amino acid sequence having a deletion, substitution and/or addition of one or more amino acids relative to the amino acid sequence of the protein defined in (L3) above, the protein having enzyme activity specified in EC2.3.3.13 and resistance to feedback inhibition of enzyme activity by L-leucine, with the proviso that the amino acid substitutions of G530D, G532D and A535T are maintained, and the protein.

51. The method according to claim 30, wherein the amino acid dehydrogenase has catalytic activity for the reaction represented by the reaction formula (IX-1) below or has enzyme activity defined in EC1.4.1.9
$$4 - methyl - 2 - oxopentanoic acid + {NH}_{3} + NADH + {H}^{+} ⇔ L - leucine + {H}_{2} O + {NAD}^{+} . \qquad ( IX - 1 )$$

52. The method according to claim 30, wherein, in step (p), the recombinant microorganism is cultured under aerobic conditions in the predetermined culture medium (X) to thereby produce L-leucine.

53. A recombinant L-leucine-producing microorganism, comprising in a forcibly expressible manner:
(i) a gene encoding a mutant type acetolactate synthase that has resistance to feedback inhibition of enzyme activity by at least one of valine, leucine and isoleucine;
(iv) a gene encoding a mutant type 2-isopropylmalate synthase that has resistance to feedback inhibition of enzyme activity by L-leucine; and
(ix) a gene encoding an amino acid dehydrogenase having at least catalytic activity on an L-leucine production reaction,
wherein an ilvE gene or an ortholog thereof present on the genome has been inactivated or deficient in the recombinant microorganism.

54. The recombinant microorganism according to claim 53, wherein the amino acid dehydrogenase has catalytic activity for the reaction represented by the following reaction formula (IX-1) or (IX-2) or has enzyme activity defined in EC1.4.1.9:
$$4 - methyl - 2 - oxopentanoic acid + {NH}_{3} + NADH + {H}^{+} ⇔ L - leucine + {H}_{2} O + {NAD}^{+} \qquad ( IX - 1 )$$
$$4 - methyl - 2 - oxopentanoic acid + {NH}_{3} + NADH + {H}^{+} ⇔ L - leucine + {H}_{2} O + {NADP}^{+} . \qquad ( IX - 2 )$$

55. The recombinant microorganism according to claim 53, wherein the gene encoding the amino acid dehydrogenase is an exogenous or heterologous gene encoding a bacterial amino acid dehydrogenase having enzyme activity defined in EC1.4.1.9, and the bacterial amino acid dehydrogenase consists of an amino acid sequence having at least one of amino acid substitutions of (a), (b) and (c) below based on the amino acid sequence shown in SEQ ID No. 2, or having at least one of amino acid substitutions of (d), (e), (f) and (g) below based on the amino acid sequence shown in SEQ ID No. 26:
(a) an amino acid substitution of an amino acid corresponding to the 51 st leucine (L) with lysine (K),
(b) an amino acid substitution of an amino acid corresponding to the 124th glycine (G) with serine(S),
(c) an amino acid substitution of an amino acid corresponding to the 310th valine (V) with alanine (A), serine(S) or glycine (G),
(d) an amino acid substitution of an amino acid corresponding to the 40th leucine (L) with lysine (K),
(e) an amino acid substitution of an amino acid corresponding to the 113th alanine (A) with glycine (G),
(f) an amino acid substitution of an amino acid corresponding to the 291st valine (V) with leucine (L),
(g) an amino acid substitution of an amino acid corresponding to the 294th valine (V) with alanine (A), serine(S) or glycine (G),
with the proviso that, in each of (a) to (g) above, the amino acid before substitution and the amino acid after substitution, when present, are different from each other.

56. The recombinant microorganism according to claim 53, wherein the gene encoding the amino acid dehydrogenase is an exogenous or heterologous gene encoding a bacterial amino acid dehydrogenase having enzyme activity defined in EC1.4.1.9, and the bacterial amino acid dehydrogenase consists of an amino acid sequence shown in any one of the following (A) to (F):
(A) an amino acid sequence shown in any one of SEQ ID Nos. 2 to 8, 10, 12 to 16, 18 to 22, 24, 26 to 30 and 32;
(B) an amino acid sequence having at least one of amino acid substitutions of (a), (b) and (c) below based on the amino acid sequence shown in SEQ ID No. 2, or having at least one of amino acid substitutions of (d), (e), (f) and (g) below based on the amino acid sequence shown in SEQ ID No. 26, relative to the amino acid sequence shown in any one of SEQ ID Nos. 2 to 8, 10, 12 to 16, 18 to 22, 24, 26 to 30 and 32,
(a) an amino acid substitution of an amino acid corresponding to the 51 st leucine (L) with lysine (K),
(b) an amino acid substitution of an amino acid corresponding to the 124th glycine (G) with serine(S),
(c) an amino acid substitution of an amino acid corresponding to the 310th valine (V) with alanine (A), serine(S) or glycine (G),
(d) an amino acid substitution of an amino acid corresponding to the 40th leucine (L) with lysine (K),
(e) an amino acid substitution of an amino acid corresponding to the 113th alanine (A) with glycine (G),
(f) an amino acid substitution of an amino acid corresponding to the 291st valine (V) with leucine (L),
(g) an amino acid substitution of an amino acid corresponding to the 294th valine (V) with alanine (A), serine(S) or glycine (G),
with the proviso that, in each of (a) to (g) above, the amino acid before substitution and the amino acid after substitution, when present, are different from each other;
(C) an amino acid sequence having a deletion, substitution and/or addition of one or more amino acids relative to the amino acid sequence set forth in (A) or (B) above; and
(D) an amino acid sequence having at least 60% sequence identity to the amino acid sequence set forth in (A) or (B) above,
with the proviso that, in each of the amino acid sequences set forth in (C) and (D) above, the amino acid substitution(s) set forth in (B) above, when present, are maintained.

57. The recombinant microorganism according to claim 53, wherein the gene encoding the amino acid dehydrogenase is an exogenous or heterologous gene encoding a bacterial amino acid dehydrogenase having enzyme activity defined in EC1.4.1.9, and the bacterial amino acid dehydrogenase consists of an amino acid sequence shown in any one of the following (A) to (F):
(A) an amino acid sequence shown in any one of SEQ ID Nos. 2, 10, 18 or 26;
(B) an amino acid sequence having at least one of amino acid substitutions of (a), (b) and (c) below based on the amino acid sequence shown in SEQ ID No. 2, or having at least one of amino acid substitutions of (d), (e), (f) and (g) below based on the amino acid sequence shown in SEQ ID No. 26, relative to the amino acid sequence shown in any one of SEQ ID Nos. 2, 10, 18 or 26,
(a) an amino acid substitution of an amino acid corresponding to the 51st leucine (L) with lysine (K),
(b) an amino acid substitution of an amino acid corresponding to the 124th glycine (G) with serine(S),
(c) an amino acid substitution of an amino acid corresponding to the 310th valine (V) with alanine (A), serine(S) or glycine (G),
(d) an amino acid substitution of an amino acid corresponding to the 40th leucine (L) with lysine (K),
(e) an amino acid substitution of an amino acid corresponding to the 113th alanine (A) with glycine (G),
(f) an amino acid substitution of an amino acid corresponding to the 291st valine (V) with leucine (L),
(g) an amino acid substitution of an amino acid corresponding to the 294th valine (V) with alanine (A), serine(S) or glycine (G),
with the proviso that, in each of (a) to (g) above, the amino acid before substitution and the amino acid after substitution, when present, are different from each other;
(C) an amino acid sequence having a deletion, substitution and/or addition of one or more amino acids relative to the amino acid sequence set forth in (A) or (B) above; and
(D) an amino acid sequence having at least 60% sequence identity to the amino acid sequence set forth in (A) or (B) above,
with the proviso that, in each of the amino acid sequences set forth in (C) and (D) above, the amino acid substitution(s) set forth in (B) above, when present, are maintained.

58. The recombinant microorganism according to claim 53, wherein the gene encoding the amino acid dehydrogenase is an exogenous or heterologous gene encoding a bacterial amino acid dehydrogenase having enzyme activity defined in EC1.4.1.9, and the bacterial amino acid dehydrogenase consists of an amino acid sequence shown in any one of the following (G1) to (I):
(G1) an amino acid sequence shown in SEQ ID No. 10 or 18;
(G2) an amino acid sequence having an amino acid substitution of V310A or V310C relative to the amino acid sequence shown in SEQ ID No. 2;
(G3) an amino acid sequence having a combination of amino acid substitutions of G124S and V310A or V310G relative to the amino acid sequence shown in SEQ ID No. 2;
(G4) an amino acid sequence having a combination of amino acid substitutions of L40K, A113G, V291L, and V294S relative to the amino acid sequence shown in SEQ ID No. 26;
(H) an amino acid sequence having a deletion, substitution and/or addition of one or more amino acids relative to the amino acid sequence set forth in any one of (G1) to (G4) above; and
(I) an amino acid sequence having at least 60% sequence identity to the amino acid sequence set forth in any one of (G1) to (G4) above;
with the proviso that, in each of (H) and (I) above, the amino acid substitution(s) set forth in each of (G2) to (G4) above, when present, are maintained.

59. The recombinant microorganism according to claim 58, wherein the amino acid sequence set forth in (H) above has a deletion, substitution and/or addition of 1 to 50 acids relative to the amino acid sequence set forth in any one of (G1) to (G4) above, and the amino acid sequence set forth in (I) above has at least 85% sequence identity to the amino acid sequence set forth in any one of (G1) to (G4) above.

60. The recombinant microorganism according to claim 59, wherein the amino acid sequence set forth in (G1) above is the amino acid sequence shown in SEQ ID No. 18.

61. The recombinant microorganism according to claim 53, wherein the recombinant microorganism is a bacterium.

62. The recombinant microorganism according to claim 53, wherein the recombinant microorganism is a coryneform bacterium.

63. The recombinant microorganism according to claim 60, wherein the recombinant microorganism is a bacterium.

64. The recombinant microorganism according to claim 60, wherein the recombinant microorganism is a coryneform bacterium.
